SEQUENCE LISTING (7 sequences)

SEQ ID NO: 1 - DNA, 4421 bp, synthetic construct
  source 1..4421: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgta

SEQ ID NO: 2 - protein, 1473 aa, unidentified
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNATNVSNNSNTSNDSNVSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDL

SEQ ID NO: 3 - DNA, 1032 bp, synthetic construct
  source 1..1032: mol_type = other DNA
gccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcg

SEQ ID NO: 4 - DNA, 1653 bp, synthetic construct
  source 1..1653: mol_type = other DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 5 - DNA, 5175 bp, synthetic construct
  source 1..5175: mol_type = other DNA
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 6 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 7 - DNA, 1428 bp, synthetic construct
  source 1..1428: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

(19) United States
(12) Patent Application Publication
(10) Pub. No.: US 2025/0195923 A1
(43) Pub. Date: Jun. 19, 2025

(54) METHODS AND SYSTEMS FOR IMPROVED DELIVERY VIA ULTRASOUND

(71) Applicant: SonoThera, Inc., South San Francisco, CA (US)

(72) Inventors: Steven B. FEINSTEIN, Highland Park, IL (US); Kenneth GREENBERG, Berkeley, CA (US); Barry Campbell, San Mateo, CA (US)

(21) Appl. No.: 18/981,179

(22) Filed: Dec. 13, 2024

Related U.S. Application Data
(60) Provisional application No. 63/610,397, filed on Dec. 14, 2023.
(60) Provisional application No. 63/625,272, filed on Jan. 25, 2024.
(60) Provisional application No. 63/625,275, filed on Jan. 25, 2024.
(60) Provisional application No. 63/656,394, filed on Jun. 5, 2024.
(60) Provisional application No. 63/711,148, filed on Oct. 23, 2024.

(51) Int. Cl. A61N 7/00; A61K 41/00; A61K 48/00

(52) U.S. Cl. CPC A61N 7/00; A61K 41/0028; A61K 48/0075

(57) ABSTRACT

Provided are systems and methods of delivering an exogenous payload to a cell in a tissue of a subject, including: administering an exogenous payload to the subject; administering a sonoactive agent to the subject, and applying an acoustic radiation force (ARF) to the subject, thereby generating shear waves in the tissue of the subject, wherein the acoustic radiation force enhances delivery of the exogenous payload to the cell in the tissue of an organ of the subject.

### CROSS REFERENCE TO RELATED APPLICATIONS

This application claims the benefit of U.S. Provisional Patent Application No. 63/610,397 filed 14 Dec. 2023, and U.S. Provisional Patent Application No. 63/625,272 filed 25 Jan. 2024, U.S. Provisional Patent Application No. 63/625,275 filed 25 Jan. 2024, U.S. Provisional Patent Application No. 63/656,394 filed 5 Jun. 2024, and U.S. Provisional Patent Application No. 63/711,148 filed 23 Oct. 2024 each of which is incorporated herein by reference in its entirety and for all purposes.

### INCORPORATION BY REFERENCE OF SEQUENCE LISTING

The present application is being filed along with a Sequence Listing in electronic format. The Sequence Listing is provided as a file entitled 62668-731601.XML, created Dec. 10, 2024, which is 22,779 bytes in size. The information in the electronic format of the Sequence Listing is incorporated by reference in its entirety.

### BACKGROUND

Gene therapy, in which a functional copy of a gene is transfected into a cell, has been proposed as a possible method of treating genetic diseases. However, prior art methods of gene therapy using ultrasound or sonoporation suffer from significant shortcomings such as low transfection rates and insufficient gene expression, which have prevented the clinical development and commercialization of these methodologies. There remains a need in the art for an effective gene therapy technique that can transfect a gene to a cell in an organ or a tissue in a subject in a safe, effective, and durable manner.

### SUMMARY

One approach to increasing the efficiency of ultrasound based nucleic acid delivery techniques has been to increase the power of the ultrasound, for example, using high-intensity focused ultrasound (HIFU). However, HIFU also presents significant safety concerns to patients due to the potential for tissue ablation and cell death occurring from thermal effects in tissue, as well cavitation effects due to interaction of HIFU with sonoactive agents. For instance, HIFU is FDA approved to treat essential tremor and parkinsonian tremor by tissue ablation generating selective and precise thermal lesions in the brain. Other ultrasound based nucleic acid delivery techniques which utilize lower intensity ultrasound more commonly associated with imaging have struggled to consistently achieve high levels of gene expression, uniform tissue biodistribution, and tissue penetration in large enough regions to render the treatment clinically meaningful, and are not generally considered efficacious. There is a lack of a reliable and effective ultrasound based delivery technique suitable for delivery of payloads in most tissue types which is also tolerable and repeatable in most subjects. Disclosed herein are methods of sonoporation in which an exogenous payload is delivered to cells in a tissue of a subject using an acoustic radiation force generated when applying ultrasound which is highly effective at transfecting cells throughout in the target organ, even those remote from the ultrasound energy source, while also minimizing risk to the subject in a highly tolerable and repeatable ultrasound based procedure. Aspects of the sonoporation methods disclosed herein may include inducing displacing the tissue of the subject with an acoustic radiation force to induce propagation of shear waves throughout the tissue of the target organ of the subject, thereby enhancing the biodistribution of the payload throughout the target organ. Aspects of the sonoporation methods disclosed herein may include applying an acoustic radiation force to an organ and transmitting an acoustic pressure wave through the vasculature of the organ displacing the sonoactive agents from the lumen of the blood vessels towards the vessel wall and inducing stable vibrational and inertial cavitation of substantially all of the sonoactive agents in the treated organ, thereby enhancing the delivery of the payload throughout the target organ. As shown and described herein, methods of sonoporation in which using acoustic radiation force to induce propagation of shear waves in a tissue of a subject and substantially disrupt the sonoactive agents through the entire organ and significantly enhances payload delivery and gene expression as compared to low intensity ultrasound techniques by using increased acoustic output, without significantly reducing the tolerability of the procedure by the subject in the target organ or tissue as tends to occur with application of HIFU. In some cases, the method may further include applying the acoustic radiation force to induce propagation of shear waves throughout the tissue in combination with other secondary ultrasound energies such as imaging ultrasound including plane wave ultrasound or B-mode ultrasound to visualize and direct the ultrasound procedure.

Aspects disclosed herein provide a method of distributing a payload across an organ, comprising: administering the payload and a sonoactive agent to the subject, and applying an acoustic radiation force (ARF) sufficient to deliver at least 1 copy per nanogram throughout the organ. Aspects disclosed herein provide a method of delivering an exogenous payload to a cell in a tissue of a subject, the method comprising: delivering an exogenous payload to the subject; and applying an acoustic radiation force (ARF) to the subject, thereby generating shear waves in the tissue of the subject, wherein the acoustic radiation force enhances delivery of the exogenous payload to the cell in the tissue of an organ of the subject. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at an ultrasound spatial peak temporal average intensity of 100-5000 mW/cm2. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy having a pulse length of greater than 200 μs (microseconds) and at an ultrasound spatial peak temporal average intensity of up to 5000 mW/cm2. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at a duty cycle of less than 5% and a pulse length of greater than 200 μs (microseconds). Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at an ultrasound spatial peak temporal average intensity of at least 100 mW/cm2 and duty cycle of less than 1%. In some embodiments, the ARF is applied at an ultrasound spatial peak temporal average intensity of 100-5000 mW/cm2.

In some embodiments, the ARF is applied at an ultrasound pulse length of at least 200 us. In some embodiments, the ARF is applied at duty cycle of less than 5%. In some embodiments, the ARF is applied at duty cycle of less than 4, 3, 2, or 1.5%. In some embodiments, the ARF is applied at an ultrasound pulse length of at least 20 us. In some embodiments, the payload is an exogenous payload, and wherein the ARF generates shear waves in a tissue of the organ of the subject. In some embodiments, the payload is expressed in the cell at an expression level of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some embodiments, the payload is expressed throughout the organ in every lobe of the organ. In some embodiments, the payload is expressed throughout the organ in two samples of the organ taken from opposite ends of the organ. In some embodiments, the samples are samples sized up to 1 cm³ or up to 1 g. In some embodiments, the payload is expressed in the cell at an expression level of at least 0.005 copies per diploid genome. In some embodiments, the payload is expressed in the cell at an expression level of at least 0.01 copies per diploid genome. In some embodiments, the ARF is applied at an ultrasound intensity of at least 150, 175, 200, 225, 250, 275, 300, 350, 400, 450, 500, 600, 700,800, 900, 1000, 1250, 1500, 1750, 2000, 2250, 2500, 2750, 3000, 3250, 3500, 3750, 4000, 4250, 4500, or 4750 mW/cm2. In some embodiments, the ARF is applied at an ultrasound intensity of up to 500, 600, 700,800, 900, 1000, 1250, 1500, 1750, 2000, 2250, 2500, 2750, 3000, 3250, 3500, 3750, 4000, 4250, 4500, 4750, or 5000 mW/cm2. In some embodiments, the applying ultrasonic acoustic energy to the tissue comprises applying the ultrasound acoustic energy at an ultrasound intensity of at least 100 mW/cm². In some embodiments, the applying ultrasonic acoustic energy to the tissue comprises applying the ultrasound acoustic energy at an ultrasound intensity of about 100 mW/cm² to about 10,000 mW/cm². In some embodiments, the applying ultrasonic acoustic energy to the tissue comprises applying the ultrasound acoustic energy at an ultrasound intensity of about 100 mW/cm² to about 5,000 mW/cm². In some embodiments, the applying ultrasonic acoustic energy to the tissue comprises applying the ultrasound acoustic energy at an ultrasound intensity of up to 5,000 mW/cm². In some embodiments, the applying ultrasonic acoustic energy to the tissue comprises applying the ultrasound acoustic energy at an ultrasound intensity of about 100 mW/cm² to about 500 mW/cm². In some embodiments, the applying ultrasonic acoustic energy to the tissue comprises applying the ultrasound acoustic energy at an ultrasound intensity of about 110 mW/cm² to about 200 mW/cm². In some embodiments, the applying ultrasonic acoustic energy to the tissue comprises applying the ultrasound acoustic energy at an ultrasound intensity of about 188 mW/cm². In some embodiments, the ultrasound intensity is a spatial-peak temporal average intensity (Ispta). In some embodiments, the ARF is applied at an ultrasound pulse length of at least 20 us. The method any one of claims 1, 4, or 5, wherein a pulse length is about 100 microseconds to about 500 microseconds. In some embodiments, the ARF is applied at an ultrasound pulse length 200-5000 us. In some embodiments, the ARF is applied at an ultrasound pulse length of at least 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us. In some embodiments, the ARF is applied at an ultrasound pulse length of up to 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us. In some embodiments, the ARF is a focused acoustic radiation force. In some embodiments, the acoustic radiation force is applied in two or more pulses, with an interval between each of the two or more pulses. In some embodiments, a plane wave ultrasound is applied to the tissue during the interval. In some embodiments, a B-Mode ultrasound is applied to the tissue during the interval. In some embodiments, the interval is up to 500 milliseconds. In some embodiments, the interval is up to 100, 500, 1000, 1500, 2000, 2500, 3000, 4000, or 5000 milliseconds. In some embodiments, the interval is from about 100 milliseconds to about 5000 milliseconds. In some embodiments, a sum of a pulse duration of the two or more pulses and the interval comprises a pulse repetition period. In some embodiments, the pulse repetition period is at least 5 milliseconds (ms). In some embodiments, the pulse repetition period is up to 5000 ms. In some embodiments, the pulse repetition period is 5-5000 ms. In some embodiments, the pulse repetition period is 20-2000 ms. In some embodiments, the pulse repetition period is 1000-2000 ms. In some embodiments, the pulse repetition period is 100-5000 ms. In some embodiments, the acoustic radiation force is applied at a pulse repetition frequency of 0.05 to 500 Hz. In some embodiments, the acoustic radiation force is applied at a pulse repetition frequency of 0.05 to 250 Hz In some embodiments, the acoustic radiation force is applied at a pulse repetition frequency of 0.1 to 100 Hz. In some embodiments, the acoustic radiation force is applied at a pulse repetition frequency of 0.1 to 50 Hz. In some embodiments, the acoustic radiation force is applied at a pulse repetition frequency of 0.5 to 50 Hz. method of any one of the preceding claims, wherein the acoustic radiation force is applied at a pulse repetition frequency of 0.5 to 5 Hz. In some embodiments, the ARF deforms the tissue of the subject. In some embodiments, the ultrasonic acoustic energy is applied at a mechanical index of greater than 0.4. In some embodiments, the ultrasonic acoustic energy is applied at a mechanical index of about 1.4. In some embodiments, the ultrasonic acoustic energy is applied at a mechanical index of at least 1.3. In some embodiments, the ultrasonic acoustic energy is applied at a mechanical index of greater than 0.4 up to about 3.0. In some embodiments, the ultrasonic acoustic energy is applied at a frequency of about 0.1 MHz to about 20 MHz. In some embodiments, the ultrasonic acoustic energy is applied at a frequency of about 1 MHz to about 20 MHz. In some embodiments, the ultrasonic acoustic energy is applied at a frequency of about 0.1 MHz to about 10 MHz. In some embodiments, the ultrasonic acoustic energy is applied at a frequency of about 18 MHz. In some embodiments, the ultrasonic acoustic energy is applied at a frequency of about 2.5 MHz. In some embodiments, the applying ultrasonic acoustic energy to the tissue comprises applying the ultrasound acoustic energy at duty cycle of up to 0.1%. In some embodiments, the applying ultrasonic acoustic energy to the tissue comprises applying the ultrasound acoustic energy at duty cycle of 0.01%-1.0%. In some embodiments, the applying ultrasonic acoustic energy to the tissue comprises applying the ultrasound acoustic energy at duty cycle of 0.05%-1.0%. In some embodiments, the sonoactive microstructure do not encapsulate the exogenous payload, optionally, wherein the exogenous payload is a nucleic acid. In some embodiments, the ARF is applied with ultrasound acoustic energy at a mechanical index of at least 1.9. In some embodiments, the ARF is applied with ultrasound acoustic energy is applied at a mechanical index of at least 2.1. In some embodiments, the ARF is applied at a thermal index of less than 1.0. In some embodiments, the ARF is applied at a thermal index of 0.01-1.0. In some embodiments, the ARF is applied at a thermal index of 0.1-1.0. In some embodiments, the application of the ARF does not increase the temperature of the tissue by more than 1 C. In some embodiments, the application of the ARF does not increase the temperature of the tissue by more than 0.5 C. In some embodiments, the application of the ARF does not increase the temperature of the tissue by more than 0.1 C. In some embodiments, the ARF displaces the tissue of the subject. In some embodiments, the tissue is displaced by at least 0.001 mm. In some embodiments, the tissue is displaced by at least 0.01 mm. In some embodiments, the tissue is displaced by at least 0.1 mm. In some embodiments, the tissue is displaced by at least 1 mm. In some embodiments, the tissue is displaced by 0.01-1 mm. In some embodiments, the shear waves displace the tissue of the subject. In some embodiments, the shear waves displace the tissue of the subject in a direction perpendicular to the direction of the application of the ultrasound acoustic energy. In some embodiments, the shear waves displace the tissue by at least 0.001 mm. In some embodiments, the shear waves displace the tissue by at least 0.01 mm. In some embodiments, the shear waves displace the tissue by 0.01-1 mm. In some embodiments, a displacement of the shear waves in the tissue is by at least 0.001 mm. In some embodiments, a displacement of the shear waves in the tissue is by at least 0.01 mm. In some embodiments, a displacement of the shear waves in the tissue is by 0.01-1 mm. In some embodiments, the ARF is applied using ultrasound acoustic energy having a pulse length of greater than 200 μs (microseconds) and induces a peak negative pressure of at least 3,000 kPa in the tissue. In some embodiments, the ARF is applied using ultrasound acoustic energy at an ultrasound intensity of at least 100 mW/cm2 and a pulse length of greater than 20 μs (microseconds). In some embodiments, the ARF is applied using ultrasound acoustic energy at an ultrasound intensity of at least 100 mW/cm2 and a duty cycle of less than 4, 3, 2, 1, or 0.5%. In some embodiments, the ARF is applied using ultrasound acoustic energy having a pulse length of greater than 200 μs (microseconds) and induces a peak negative pressure of at least 3300, 3500, 4000, 4500, 5000, 5500, 6000, 6500, 7000, or 7500 kPa in the tissue. In some embodiments, the exogenous payload comprises a nucleic acid construct. In some embodiments, at least 10 mg of the nucleic acid construct is administered to the subject. In some embodiments, the exogenous payload comprises an antibody, a small molecule drug, a protein, a cell therapy, a viral vector, or a peptide. In some embodiments, the nucleic acid construct comprises a therapeutic transgene, a DNA, ASO, a snRNA, an miRNA, an mRNA, a circular RNA, a self-amplifying RNA, an siRNA, a shRNA, a gRNA, a nucleic acid encoding a TALEN, a molecule encoding a zinc-finger nuclease, donor DNA, a transposase, an integrase, a retrotransposase, a saliogase, a base editor, a prime editor, a molecule encoding a CRISPR-associated protein 9, or a nucleic acid comprising a Clustered Regularly Interspaced Short Palindromic Repeats (CRISPR) sequence. In some embodiments, the nucleic acid construct comprises a therapeutic transgene, wherein the therapeutic transgene encodes Factor VIII, Factor IX, COL4A3, COL4A4, COL4A5, PKD1, or PKD2. In some embodiments, the organ is a liver, kidney, heart, brain, pancreas, skeletal muscle, smooth muscle, bone, or brain. In some embodiments, the cell is a hepatic cell, renal cell, pancreatic cell, myocyte, cardiac cell, blood cell, a hematopoietic cell, a tumor cell, a neuron, a glial cell, an immune cell, or a skeletal muscle cell. In some embodiments, the acoustic radiation force is applied using an ultrasound probe applying ultrasound acoustic energy to the tissue. In some embodiments, the sonoactive agent comprises a plurality of sonoactive microstructures. The method any one of the preceding claims, wherein the ultrasound probe comprises a plurality of piezoelectric elements configured to emit ultrasound acoustic energy. In some embodiments, separate portions of the plurality of the piezoelectric elements each emit an ultrasound beam, wherein the acoustic radiation force (ARF) is applied using a plurality of ultrasound beams. In some embodiments, the acoustic radiation force is applied using a plurality of ultrasound beams. In some embodiments, the plurality of ultrasound beams produce a plurality of shear waves in the tissue, wherein at least two of the plurality of shear waves each originate at a different location in the tissue. In some embodiments, a first shear wave of the plurality of shear waves in the tissue constructively interferes with a second shear wave of the plurality of shear waves in the tissue. In some embodiments, applying the acoustic radiation force induces inertial cavitation of a portion of the plurality of sonoactive microstructures. In some embodiments, inducing inertial cavitation of a portion of the plurality of sonoactive microstructures during propagation of the shear wave in the tissue increases delivery of the exogenous payload to the cell. In some embodiments, the plurality of sonoactive microstructures comprise a protein-stabilized microstructure. In some embodiments, the plurality of sonoactive microstructures comprise a phospholipid stabilized microstructure. In some embodiments, the plurality of sonoactive microstructures are non-phase-shiftable microstructures. In some embodiments, the acoustic radiation force is applied for at least 10, 20, 30, 60, 120, 180, 240, 300, 360, 420, 480, 540, or 600 seconds. In some embodiments, the acoustic radiation force is applied for up to 30, 45, 60, 75, or 90 minutes. In some embodiments, the acoustic radiation force is applied for 10-600 seconds. In some embodiments, the acoustic radiation force is applied for 120-600 seconds. In some embodiments, applying ultrasonic acoustic energy to the tissue comprises applying the ultrasound acoustic energy at a focal depth of up to 10, 8, 6, or 4 cm from the ultrasound transducer. In some embodiments, applying ultrasonic acoustic energy to the tissue comprises applying the ultrasound acoustic energy at a focal depth of about 4 to about 10 cm from the ultrasound transducer. In some embodiments, applying ultrasonic acoustic energy to the tissue comprises applying the ultrasound acoustic energy at a focal depth of about 4 cm from the ultrasound transducer. In some embodiments, applying ultrasonic acoustic energy to the tissue comprises applying the ultrasound acoustic energy at a focal depth of about 6 cm from the ultrasound transducer. In some embodiments, the method further includes applying a plane wave ultrasound to the tissue. In some embodiments, applying plane wave ultrasound comprises delivering ultrasound acoustic energy to the tissue at a plurality of angles simultaneously. In some embodiments, the method further includes imaging the tissue with the plane wave ultrasound. In some embodiments, imaging the tissue comprises tracking a propagation speed of the shear waves in the tissue. In some embodiments, the plane wave ultrasound is applied at an MI of greater than 0.4. In some embodiments, the plane wave ultrasound is applied at an MI of about 1.4. In some embodiments, the plane wave ultrasound is applied at an MI of greater than 0.4 up to about 3.0. In some embodiments, the plane wave ultrasound is applied at a frequency of at least 0.1 MHz. In some embodiments, the plane wave ultrasound is applied at a frequency of about 0.1 MHz to about 10 MHz. In some embodiments, the plane wave ultrasound is applied at a frequency of about 2.5 MHz. In some embodiments, applying the plane wave ultrasound results in moving the sonoactive microstructures towards an endothelial border of the tissue comprising the cell. In some embodiments, the method further includes applying a B-mode ultrasound acoustic energy to the tissue. In some embodiments, applying the B-mode ultrasound acoustic energy comprises delivering ultrasound acoustic energy to the tissue at a plurality of angles simultaneously. In some embodiments, the method further includes imaging the tissue with the B-mode ultrasound acoustic energy. In some embodiments, imaging the tissue comprises tracking a propagation speed of the shear waves in the tissue. In some embodiments, the B-mode ultrasound acoustic energy is applied at an MI of greater than 0.4. In some embodiments, the B-mode ultrasound acoustic energy is applied at an MI of about 1.4. In some embodiments, the B-mode ultrasound acoustic energy is applied at an MI of greater than 0.4 up to about 3.0. In some embodiments, the B-mode ultrasound acoustic energy is applied at a frequency of at least 0.1 MHz. In some embodiments, the B-mode ultrasound acoustic energy is applied at a frequency of about 0.1 MHz to about 20 MHz. In some embodiments, the B-mode ultrasound acoustic energy is applied at a frequency of about 0.1 MHz to about 10 MHz. In some embodiments, the B-mode ultrasound acoustic energy is applied at a frequency of about 2.5 MHz. In some embodiments, applying the B-mode ultrasound acoustic energy results in moving the sonoactive microstructures towards an endothelial border of the tissue comprising the cell. In some embodiments, the shear waves induce inertial cavitation the sonoactive microstructures at an endothelial border of the tissue comprising the cell, thereby enhancing delivery of the nucleic acid to the cell. In some embodiments, applying the ultrasound results in moving the sonoactive microstructures towards an endothelial border of the tissue comprising the cell. In some embodiments, the shear waves induce inertial cavitation the sonoactive microstructures at an endothelial border of the tissue comprising the cell, thereby enhancing delivery of the nucleic acid to the cell. In some embodiments, the acoustic radiation force increases internalization of the exogenous payload in the cell. In some embodiments, the shear waves increase internalization of the exogenous payload in the cell. In some embodiments, inducing inertial cavitation the sonoactive microstructures increases internalization of the exogenous payload in the cell. In some embodiments, the ultrasound probe comprises a curved array probe, optionally, wherein the curved array probe is a C1-6 ultrasound probe. In some embodiments, the method further includes delivering a second or subsequent dose of the exogenous payload to the subject; and applying a second or subsequent acoustic radiation force (ARF) to the subject. In some embodiments, the second or subsequent dose of the exogenous payload are the same as a first dose of the exogenous payload. In some embodiments, the second or subsequent acoustic radiation force (ARF) is the same as the acoustic radiation force (ARF). In some embodiments, the second or subsequent acoustic radiation force (ARF) is applied at least 4 hours after the acoustic radiation force (ARF). In some embodiments, the second or subsequent dose of the exogenous payload is administered at least 4 hours after the first dose of the exogenous payload. In some embodiments, the second dose of the exogenous payload and the second acoustic radiation force (ARF) are administered at least 24 hours after the first dose of the exogenous payload and the first ARF. In some embodiments, delivering the second or subsequent dose of the exogenous payload to the subject; and applying the second or subsequent acoustic radiation force (ARF) to the subject enhances delivery of the exogenous payload to the cell in the tissue of an organ of the subject, or enhances delivery of the exogenous payload to the cell in the tissue of an organ of the subject relative to a single administration. In some embodiments, the tissue exhibits substantially no thermal injury, substantially no mechanical injury, and/or substantially no cell death after application of the acoustic radiation force. In some embodiments, the method further includes sedating the subject. In some embodiments, the exogenous payload comprises a nucleic acid payload encoding FVIII. In some embodiments, the method further includes delivering a second or subsequent dose of the exogenous payload to the subject; and applying a second or subsequent acoustic radiation force (ARF) to the subject, In some embodiments, a therapeutic level of FVIII is present in the subject's plasma following delivering the exogenous payload to the subject, and applying the acoustic radiation force (ARF) to the subject. In some embodiments, the therapeutic level of FVIII is at least 0.05 IU/mL. In some embodiments, the therapeutic level of FVIII is achieved within 72 hours following delivering the exogenous payload to the subject, and applying the acoustic radiation force (ARF) to the subject. I.

Aspects disclosed herein provide an exogenous payload and a sonoactive agent for use in a method of treating a subject having a genetic disorder requiring a gene therapy or a protein replacement therapy, the method comprising: administering to the subject the sonoactive agent and the exogenous payload, the exogenous payload comprising a nucleic acid for expression of the gene therapy or the protein replacement therapy; and applying an acoustic radiation force (ARF) and generating shear waves in a tissue of the subject with an ultrasound system comprising an ultrasound transducer applying ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof, thereby delivering the exogenous payload to the tissue of the subject, and inducing expression of the gene therapy or the protein replacement therapy in the tissue of the subject. Aspects disclosed herein provide an exogenous payload for use in a method of treating a subject having a genetic disorder requiring a gene therapy or a protein replacement therapy, the method comprising: administering to the subject the exogenous payload, the exogenous payload comprising a nucleic acid for expression of the gene therapy or the protein replacement therapy, and administering to the subject a sonoactive agent; and applying an acoustic radiation force (ARF) and generating shear waves in a tissue of the subject with an ultrasound system comprising an ultrasound transducer applying ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof, thereby delivering the exogenous payload to the tissue of the subject, and inducing expression of the gene therapy or the protein replacement therapy in the tissue of the subject. Aspects disclosed herein provide a sonoactive agent for use in a method of treating a subject having a genetic disorder requiring a gene therapy or a protein replacement therapy, the method comprising: administering to the subject the sonoactive agent, and administering to the subject an exogenous payload comprising a nucleic acid for expression of the gene therapy or the protein replacement therapy; and applying an acoustic radiation force (ARF) and generating shear waves in a tissue of the subject with an ultrasound system comprising an ultrasound transducer applying ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof, thereby delivering the exogenous payload to the tissue of the subject, and inducing expression of the gene therapy or the protein replacement therapy in the tissue of the subject. Aspects disclosed herein provide a sonoactive agent for use in a method of preparing a cell in a tissue of a subject for the delivery of an exogenous payload to the cell, the method comprising: administering a sonoactive agent to the subject; and applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof. Aspects disclosed herein provide a sonoactive agent for use in a method of preparing a cell in a tissue of a subject having been administered with a sonoactive agent for the delivery of an exogenous payload to the cell, the method comprising: applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof. Aspects disclosed herein provide a method of preparing a cell in a tissue of a subject having been administered with a sonoactive agent for the delivery of an exogenous payload to the cell, the method comprising: applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof. Aspects disclosed herein provide a method of preparing a cell in a tissue of a subject for the delivery of an exogenous payload to the cell, the method comprising: administering a sonoactive agent to the subject; and applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof. Aspects disclosed herein provide a computer-readable medium comprising instructions which, when executed by a computer, cause the computer to carry out a method of applying an acoustic radiation force with ultrasonic acoustic energy to a tissue of a subject and generating shear waves in the tissue of the subject to enhance delivery of an exogenous payload to the tissue of a subject that has been administered with the exogenous payload and a sonoactive agent, the method comprising: applying the focused ARF with an ultrasound transducer applying ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof. In some embodiments, administering the payload and the sonoactive agent to the subject, and applying the acoustic radiation force (ARF) is sufficient to deliver at least 1 copy per nanogram of subject DNA throughout the organ, optionally, wherein the payload is DNA. In some embodiments, the ARF is applied at an ultrasound intensity of 100-5000 mW/cm2. In some embodiments, the ARF is applied at an ultrasound pulse length of at least 200 us. In some embodiments, the ARF is applied at duty cycle of less than 5%. In some embodiments, the ARF is applied at duty cycle of less than 4, 3, 2, or 1.5%. In some embodiments, the ARF is applied at an ultrasound pulse length of at least 20 us. In some embodiments, the payload is an exogenous payload, and wherein the ARF generates shear waves in a tissue of the organ of the subject. In some embodiments, the payload is expressed in the cell at an expression level of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some embodiments, the payload is expressed throughout the organ in every lobe of the organ. In some embodiments, the payload is expressed throughout the organ in two samples of the organ taken from opposite ends of the organ. In some embodiments, the samples are samples sized up to 1 cm³ or up to 1 g. In some embodiments, the payload is expressed in the cell at an expression level of at least 0.005 copies per diploid genome. In some embodiments, the payload is expressed in the cell at an expression level of at least 0.01 copies per diploid genome. In some embodiments, the ARF is applied at an ultrasound intensity of at least 150, 175, 200, 225, 250, 275, 300, 350, 400, 450, 500, 600, 700, 800, 900, 1000, 1250, 1500, 1750, 2000, 2250, 2500, 2750, 3000, 3250, 3500, 3750, 4000, 4250, 4500, or 4750 mW/cm2. In some embodiments, the ARF is applied at an ultrasound intensity of up to 500, 600, 700, 800, 900, 1000, 1250, 1500, 1750, 2000, 2250, 2500, 2750, 3000, 3250, 3500, 3750, 4000, 4250, 4500, 4750, or 5000 mW/cm2. In some embodiments, the applying ultrasonic acoustic energy to the tissue comprises applying the ultrasound acoustic energy at an ultrasound intensity of at least 100 mW/cm². In some embodiments, the applying ultrasonic acoustic energy to the tissue comprises applying the ultrasound acoustic energy at an ultrasound intensity of about 100 mW/cm² to about 10,000 mW/cm². In some embodiments, the applying ultrasonic acoustic energy to the tissue comprises applying the ultrasound acoustic energy at an ultrasound intensity of about 100 mW/cm² to about 5,000 mW/cm². In some embodiments, the applying ultrasonic acoustic energy to the tissue comprises applying the ultrasound acoustic energy at an ultrasound intensity of up to 5,000 mW/cm². In some embodiments, the applying ultrasonic acoustic energy to the tissue comprises applying the ultrasound acoustic energy at an ultrasound intensity of about 100 mW/cm² to about 500 mW/cm². In some embodiments, the applying ultrasonic acoustic energy to the tissue comprises applying the ultrasound acoustic energy at an ultrasound intensity of about 110 mW/cm² to about 200 mW/cm². In some embodiments, the applying ultrasonic acoustic energy to the tissue comprises applying the ultrasound acoustic energy at an ultrasound intensity of about 188 mW/cm². In some embodiments, the ultrasound intensity is a spatial-peak temporal average intensity (Ispta). In some embodiments, the ARF is applied at an ultrasound pulse length of at least 20 us. The method any one of embodiments 1, 4, or 5, wherein a pulse length is about 100 microseconds to about 500 microseconds. In some embodiments, the ARF is applied at an ultrasound pulse length 200-5000 us. In some embodiments, the ARF is applied at an ultrasound pulse length of at least 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us. In some embodiments, the ARF is applied at an ultrasound pulse length of up to 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us. In some embodiments, the ARF is an acoustic radiation force. In some embodiments, the acoustic radiation force is applied in two or more pulses, with an interval between each of the two or more pulses. In some embodiments, a plane wave ultrasound is applied to the tissue during the interval. In some embodiments, a B-Mode ultrasound is applied to the tissue during the interval. In some embodiments, the interval is up to 500 milliseconds. In some embodiments, the interval is up to 100, 500, 1000, 1500, 2000, 2500, 3000, 4000, or 5000 milliseconds. In some embodiments, the interval is from about 100 milliseconds to about 5000 milliseconds. In some embodiments, a sum of a pulse duration of the two or more pulses and the interval comprises a pulse repetition period. In some embodiments, the pulse repetition period is at least 5 milliseconds (ms). In some embodiments, the pulse repetition period is up to 5000 ms. In some embodiments, the pulse repetition period is 5-5000 ms. In some embodiments, the pulse repetition period is 20-2000 ms. In some embodiments, the pulse repetition period is 1000-2000 ms. In some embodiments, the pulse repetition period is 100-5000 ms. In some embodiments, the acoustic radiation force is applied at a pulse repetition frequency of 0.05 to 500 Hz. In some embodiments, the acoustic radiation force is applied at a pulse repetition frequency of 0.05 to 250 Hz. In some embodiments, the acoustic radiation force is applied at a pulse repetition frequency of 0.1 to 100 Hz. In some embodiments, the acoustic radiation force is applied at a pulse repetition frequency of 0.1 to 50 Hz. In some embodiments, the acoustic radiation force is applied at a pulse repetition frequency of 0.5 to 50 Hz. method of any one of the preceding embodiments, wherein the acoustic radiation force is applied at a pulse repetition frequency of 0.5 to 5 Hz. In some embodiments, the ARF deforms the tissue of the subject. In some embodiments, the ultrasonic acoustic energy is applied at a mechanical index of greater than 0.4. In some embodiments, the ultrasonic acoustic energy is applied at a mechanical index of about 1.4. In some embodiments, the ultrasonic acoustic energy is applied at a mechanical index of at least 1.3. In some embodiments, the ultrasonic acoustic energy is applied at a mechanical index of greater than 0.4 up to about 3.0. In some embodiments, the ultrasonic acoustic energy is applied at a frequency of about 0.1 MHz to about 20 MHz. In some embodiments, the ultrasonic acoustic energy is applied at a frequency of about 1 MHz to about 20 MHz. In some embodiments, the ultrasonic acoustic energy is applied at a frequency of about 0.1 MHz to about 10 MHz. In some embodiments, the ultrasonic acoustic energy is applied at a frequency of about 18 MHz. In some embodiments, the ultrasonic acoustic energy is applied at a frequency of about 2.5 MHz. In some embodiments, the applying ultrasonic acoustic energy to the tissue comprises applying the ultrasound acoustic energy at duty cycle of up to 0.1%. In some embodiments, the applying ultrasonic acoustic energy to the tissue comprises applying the ultrasound acoustic energy at duty cycle of 0.01%-1.0%. In some embodiments, the applying ultrasonic acoustic energy to the tissue comprises applying the ultrasound acoustic energy at duty cycle of 0.05%-1.0%. In some embodiments, the sonoactive microstructure do not encapsulate the exogenous payload, optionally, wherein the exogenous payload is a nucleic acid. In some embodiments, the ARF is applied with ultrasound acoustic energy at a mechanical index of at least 1.9. In some embodiments, the ARF is applied with ultrasound acoustic energy is applied at a mechanical index of at least 2.1. In some embodiments, the ARF is applied at a thermal index of less than 1.0. In some embodiments, the ARF is applied at a thermal index of 0.01-1.0. In some embodiments, the ARF is applied at a thermal index of 0.1-1.0. In some embodiments, the application of the ARF does not increase the temperature of the tissue by more than 1 C. In some embodiments, the application of the ARF does not increase the temperature of the tissue by more than 0.5 C. In some embodiments, the application of the ARF does not increase the temperature of the tissue by more than 0.1 C. In some embodiments, the ARF displaces the tissue of the subject. In some embodiments, the tissue is displaced by at least 0.001 mm. In some embodiments, the tissue is displaced by at least 0.01 mm. In some embodiments, the tissue is displaced by at least 0.1 mm. In some embodiments, the tissue is displaced by at least 1 mm. In some embodiments, the tissue is displaced by 0.01-1 mm. In some embodiments, the shear waves displace the tissue of the subject. In some embodiments, the shear waves displace the tissue of the subject in a direction perpendicular to the direction of the application of the ultrasound acoustic energy. In some embodiments, the shear waves displace the tissue by at least 0.001 mm. In some embodiments, the shear waves displace the tissue by at least 0.01 mm. In some embodiments, the shear waves displace the tissue by 0.01-1 mm. In some embodiments, the ARF is applied using ultrasound acoustic energy having a pulse length of greater than 200 μs (microseconds) and induces a peak negative pressure of at least 3,000 kPa in the tissue. In some embodiments, the ARF is applied using ultrasound acoustic energy at an ultrasound intensity of at least 100 mW/cm2 and a pulse length of greater than 20 μs (microseconds). In some embodiments, the ARF is applied using ultrasound acoustic energy at an ultrasound intensity of at least 100 mW/cm2 and a duty cycle of less than 4, 3, 2, 1, or 0.5%. In some embodiments, the ARF is applied using ultrasound acoustic energy having a pulse length of greater than 200 μs (microseconds) and induces a peak negative pressure of at least 3300, 3500, 4000, 4500, 5000, 5500, 6000, 6500, 7000, or 7500 kPa in the tissue. In some embodiments, the exogenous payload comprises a nucleic acid construct. In some embodiments, at least 10 mg of the nucleic acid construct is administered to the subject. In some embodiments, the exogenous payload comprises an antibody, a small molecule drug, a protein, a cell therapy, a viral vector, or a peptide. In some embodiments, the nucleic acid construct comprises a therapeutic transgene, a DNA, ASO, a snRNA, an miRNA, an mRNA, a circular RNA, a self-amplifying RNA, an siRNA, a shRNA, a gRNA, a nucleic acid encoding a TALEN, a molecule encoding a zinc-finger nuclease, donor DNA, a transposase, an integrase, a retrotransposase, a saliogase, a base editor, a prime editor, a molecule encoding a CRISPR-associated protein 9, or a nucleic acid comprising a Clustered Regularly Interspaced Short Palindromic Repeats (CRISPR) sequence. In some embodiments, the nucleic acid construct comprises a therapeutic transgene, wherein the therapeutic transgene encodes Factor VIII, Factor IX, COL4A3, COL4A4, COL4A5, PKD1, or PKD2. In some embodiments, the organ is a liver, kidney, heart, brain, pancreas, skeletal muscle, smooth muscle, bone, or brain. In some embodiments, the cell is a hepatic cell, renal cell, pancreatic cell, myocyte, cardiac cell, blood cell, a hematopoietic cell, a tumor cell, a neuron, a glial cell, an immune cell, or a skeletal muscle cell. In some embodiments, the acoustic radiation force is applied using an ultrasound probe applying ultrasound acoustic energy to the tissue. In some embodiments, the sonoactive agent comprises a plurality of sonoactive microstructures. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1. In some embodiments, the method further includes a second or subsequent dose of the exogenous payload to the subject; and applying a second or subsequent acoustic radiation force (ARF) to the subject. In some embodiments, the second or subsequent dose of the exogenous payload are the same as a first dose of the exogenous payload. In some embodiments, the second or subsequent acoustic radiation force (ARF) is the same as the acoustic radiation force (ARF). In some embodiments, the second or subsequent acoustic radiation force (ARF) is applied at least 4 hours after the acoustic radiation force (ARF). In some embodiments, the second or subsequent dose of the exogenous payload is administered at least 4 hours after the first dose of the exogenous payload. In some embodiments, the second dose of the exogenous payload and the second acoustic radiation force (ARF) are administered at least 24 hours after the first dose of the exogenous payload and the first ARF.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 provides fluorescence data showing gene expression over time for subjects undergoing sonoporation treatment using standard B-mode imaging based ultrasound protocols.

FIG. 2 provides fluorescence data showing an increase in gene expression for subjects undergoing sonoporation treatment using an acoustic radiation force protocol as compared to subjects undergoing sonoporation treatment using standard B-mode imaging based ultrasound protocols.

FIG. 3 illustrates an exemplary ultrasound transducer system having computer processors with a computer readable medium storing instructions for implementing the methods of the present disclosure.

FIG. 4 illustrates propagation of an ultrasound wave and compressional wave through an elastic medium.

FIG. 5 illustrates propagation of a shear or secondary wave through an elastic medium.

FIG. 6 provides fluorescence data showing gene expression for subjects undergoing sonoporation treatment using an acoustic radiation force protocol as compared to subjects under sedation.

FIG. 7 provides fluorescence data showing gene expression for subjects undergoing repeated sonoporation treatments using an acoustic radiation force protocol.

FIG. 8 provides data showing gene expression as measured by a secreted protein in subjects undergoing sonoporation treatment using an acoustic radiation force protocol.

FIG. 9 provides copy number data showing gene expression in subjects undergoing sonoporation treatment using an acoustic radiation force protocol.

FIG. 10A-10G are fluorescence images showing gene expression in a treated liver of a subject undergoing sonoporation treatment using an acoustic radiation force protocol.

FIG. 11 provides fluorescence data showing gene expression in subjects undergoing sonoporation treatment using an acoustic radiation force protocol.

FIG. 12 provides fluorescence data showing gene expression in subjects undergoing sonoporation treatment using an acoustic radiation force protocol.

FIG. 13 provides copy number data showing gene expression in subjects undergoing sonoporation treatment using an acoustic radiation force protocol.

FIG. 14 provides copy number data showing gene expression in subjects undergoing sonoporation treatment using an acoustic radiation force protocol.

FIG. 15 illustrates transmission of an acoustic wave and shear waves in a treated organ and resulting sonoactive agent cavitation.

FIG. 16 provides a graph of ultrasound intensity over time illustrating application of pulsed ultrasound with various measurements of ultrasound intensity and pulse parameters identified.

FIG. 17A-17C provides histology images which compare treated and untreated regions of tissue and show a lack of adverse effect on tissue treated with the methods disclosed herein.

FIG. 18 provides copy number data showing gene expression in subjects undergoing sonoporation treatment using a focused acoustic radiation force protocol.

FIG. 19 provides copy number data showing gene expression in different regions of a treated organ for subjects undergoing sonoporation treatment using standard B-mode imaging based ultrasound protocols.

### DETAILED DESCRIPTION

One approach to increasing the efficiency of ultrasound based nucleic acid delivery techniques has been to increase the power and resulting of the ultrasound, for example, using high-intensity focused ultrasound (HIFU). However, HIFU also presents significant safety concerns to patients due to the potential for tissue ablation and cell death occurring from thermal effects in tissue, as well cavitation effects due to interaction of HIFU with sonoactive agents. Other ultrasound based nucleic acid delivery techniques which utilize lower intensity ultrasound more commonly associated with imaging have struggled to consistently achieve high levels of gene expression, and are generally not considered efficacious. There is a lack of a reliable and effective ultrasound based delivery technique suitable for delivery of payloads in most tissue types which is also safe and repeatable in most subjects. Disclosed herein are methods of sonoporation in which an exogenous payload is delivered to a cell in a tissue of a subject using a focused acoustic radiation force applied using ultrasound which is highly effective at transfecting cells in the target organ while also minimizing risk to the subject in a highly tolerable and repeatable ultrasound based procedure. As is shown and described herein, the sonoporation methods of the present disclosure deliver ultrasound to subject tissue which displacing the tissue of the subject with an acoustic radiation force to induce propagation of shear waves throughout the tissue and which transfer acoustic pressure applied form an ultrasound push pulse to hydrostatic pressure in the circulatory system, each of which significantly enhance payload delivery and gene expression as compared to low intensity ultrasound techniques, without significantly reducing the safety of the procedure to the subject in the target organ or tissue as tends to occur with application of HIFU.

Aspects of the sonoporation methods disclosed herein may include inducing displacing the tissue of the subject with an acoustic radiation force to induce propagation of shear waves throughout the tissue of the subject, thereby enhancing the biodistribution of the payload throughout the target organ. Aspects of the sonoporation methods disclosed herein may include applying an acoustic radiation force to an organ and transmitting an acoustic pressure wave through the vasculature of the organ and disrupting substantially all of the sonoactive agents in the treated organ, thereby enhancing the delivery of the payload throughout the target organ. Aspects disclosed herein provide a method of distributing a payload across an organ, comprising: administering the payload and a sonoactive agent to the subject, and applying an acoustic radiation force (ARF) sufficient to deliver at least 1 copy per nanogram of subject DNA throughout the organ, optionally, wherein the payload is DNA. Aspects disclosed herein provide a method of delivering an exogenous payload to a cell in a tissue of a subject, the method comprising: delivering an exogenous payload to the subject; administering a sonoactive agent to the subject; and applying a acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at an ultrasound intensity of 100-5000 mW/cm2. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the focused acoustic radiation force enhances delivery of the exogenous payload to the cell in the tissue of an organ of the subject, wherein the ARF is applied using ultrasound acoustic energy having a pulse length of greater than 200 μs (microseconds) and at an ultrasound intensity of up to 5000 mW/cm2. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the focused acoustic radiation force enhances delivery of the exogenous payload to the cell in the tissue of an organ of the subject, wherein the ARF is applied using ultrasound acoustic energy at a duty cycle of less than 5% and a pulse length of greater than 200 us (microseconds). Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the focused acoustic radiation force enhances delivery of the exogenous payload to the cell in the tissue of an organ of the subject, wherein the ARF is applied using ultrasound acoustic energy at an ultrasound intensity of at least 100 mW/cm2 and duty cycle of less than 1%.

Ultrasound refers to the application of acoustic energy in the range of greater than 20 kHz up to several gigahertz which propagates as a compressional wave through a medium. Ultrasound is used in many different fields, most commonly in the field of diagnostics and medical imaging for producing images of tissue within the human body. Ultrasound acoustic energy can be generated at various frequencies within the 20 kHz up to several gigahertz range, most commonly within the range of about 1 to 10 megahertz when used for diagnostic imaging purposes. Ultrasound is commonly applied using ultrasonic transducers comprising one or more piezoelectric crystals which convert electrical energy into acoustic energy. In addition to imaging applications, ultrasound can also be used for a variety of other diagnostic and therapeutic applications, including determination of tissue elasticity and fibrosis, and focused destruction of tissue using ultrasound ablation. In the case of sonoporation, administration of the ultrasound in combination with sonoactive agents can form transient pores in the epithelial tissues and cell membranes, allowing for delivery of exogenous payloads to cells.

B mode ultrasound imaging refers to brightness mode imaging, in which ultrasonic waves are reflected from the tissue of a subject back to the ultrasound probe, and displayed on a 2 dimensional display with objects that are closer to the ultrasound transducer appear brighter having generated a strong reelection, and objects which are farther away from the ultrasound transducer appear darker having generated a weaker reflection. B mode ultrasound imaging generally will focus ultrasonic acoustic energy emitted from a plurality of ultrasound arrays comprising piezoelectric crystals into a focused ultrasound beam which penetrates into the tissue about a vertical axis which is perpendicular to the surface of the ultrasound probe. The focused ultrasound beam reflects off the tissue and back towards the ultrasound transducer, forming a scan line in an ultrasound image. By moving the ultrasound transducer about a surface of tissue, an image of the underlying tissue can be generated using a B mode ultrasound image. B mode ultrasound imaging is the most common form of ultrasound used in the United States for medical imaging, and is what is commonly referred to as diagnostic or imaging ultrasound. B mode ultrasound is low intensity ultrasound typically of the order of 5-25 mW/cm2 (Ispta) and is considered to be low risk to subjects as to potential for generation of bioeffects.

Plane wave imaging refers to an ultrasound imaging technique in which a plurality of ultrasound arrays comprising piezoelectric crystals in an ultrasound transducer are simultaneously fired without directing ultrasonic acoustic energy into a focused ultrasound beam, and which instead direct a large unfocused sheet or wave of ultrasound acoustic energy into a medium or tissue underlying an ultrasound probe. The primary difference between plane wave ultrasound imaging and B mode ultrasound imaging is the number of transducer arrays which are fired. Plain wave imaging typically will fire all arrays within an ultrasound transducer Producing a much larger and less focused wave of ultrasonic energy, while B mode imaging will typically only fire a subset of arrays which focused the ultrasound into a beam producing what is commonly referred to as a scan line. In plane wave imaging, the acoustic radiation pressure is almost uniform over the entire field of view, and lower peak and negative pressures are typically experienced as compared to traditional beam mode focused ultrasound beam imaging.

Doppler ultrasound is a specialized form of ultrasound imaging that evaluates blood flow dynamics within vessels. B-mode ultrasound imaging uses ultrasound to create images of body structures based on the echoes that return as these waves encounter various tissues. Doppler ultrasound adds another layer by measuring the change in frequency—or “Doppler shift”—of the sound waves as they bounce off moving red blood cells. This shift in frequency allows the device to not only detect the presence of blood flow, but also determine its speed and direction. Doppler ultrasound can be used to assess circulatory problems, detect blockages or narrowing in arteries, evaluate heart valve function, and monitor the health of patients with vascular conditions. Doppler ultrasound can also be used to track the propagation of shear waves in tissue.

Acoustic radiation force refers to a static or transient unidirectional force applied by an acoustic wave on the propagation medium or to an object in the path of the acoustic wave which occurs through the transfer of momentum from the ultrasound to the medium. Acoustic radiation forces can be applied using an ultrasound transducer when applying ultrasonic acoustic energy to a surface of a tissue or a propagation medium with sufficient ultrasound intensity. When applying a sufficient acoustic radiation force to a propagation medium or a tissue, the propagation medium or tissue under lying the ultrasound probe may be displaced.

Shear waves, or secondary waves, refer to transversely oriented waves which occur in elastic medium that is subjected to a periodic shear. Shear refers to a change in shape without a change of volume of a layer of a propagation medium or tissue produced by a pair of equal forces acting in opposite directions about two faces of the layer or the propagation medium. Shear waves are a type of elastic wave which move through the body of an object or a propagation medium. In an elastic medium, the layer or the tissue will resume its original shape following application of the shear force, adjacent layers will undergo subsequent shear, and the movement of particles within the medium or tissue will be propagated as a shear wave throughout the propagation medium or tissue. In an elastic medium, shear waves can be produced as a secondary wave following a compressional wave which is transmitted in the propagation medium or tissue. Ultrasound applying an acoustic radiation force can apply a compressional wave to a tissue, which can result in formation of shear waves in a tissue when applied with sufficient intensity, at regular intervals, for sufficient periods of time to induce a regular shear in layers of a tissue. As is disclosed herein the application of an acoustic radiation force (ARF) can be utilized to provide a method of distributing a payload across an organ, and can enhance delivery of the exogenous payload to the cell in the tissue of the organ which is applied the ARF.

A compressional wave displaces tissue in a direction parallel to the propagation of the compressional waves. An ultrasound transducer can induce a compressional wave in a tissue which propagates from the ultrasound transducer about a vector normal to a surface of the ultrasound transducer. In some embodiments, applying the acoustic radiation force to the tissue comprises generating a compressional wave in the tissue. As an elastic tissue recovers from displacement due to a compressional wave, shear waves or secondary waves can be generated. In a shear wave, the direction of particle motion is parallel to the direction of propagation of the compressional wave, and the direction of propagation of the shear wave is normal to the direction of propagation of the compressional wave.

Shear wave elastography refers to a diagnostic technique using ultrasound to determine the elastic modulus of tissue, which is indicative of its fibrotic quality. Diseased tissue with certain fibrotic conditions will result in a significantly reduced elastic modulus of the tissue, as compared to a healthy tissue which is reasonably elastic as compared to diseased tissue in a fibrotic state. Shear wave elastography uses a combination of acoustic radiation force, plane wave imaging and/or B mode imaging to provide a clinician with information as to the fibrotic quality of a tissue. Shear wave elastography applies an acoustic radiation force to displace the tissue underlying an ultrasound probe with a compressional wave, thereby generating shear waves in the tissue, applies a plane wave or doppler ultrasound to the tissue to monitor the propagation of the shear waves throughout the tissue thereby calculating the elastic modulus, and overlays this data atop a standard B mode ultrasound image in order to provide a visual representation of tissue stiffness.

Application of ultrasound across various ultrasound regimes can be characterized by the physical properties of the acoustic waves and the medium which they are interacting with. Ultrasound as a compressional wave can be characterized by the acoustic frequency of the ultrasound wave, ranging from around 20 kHz up to several gigahertz range, most commonly within the range of about 1 to 10 megahertz when used for diagnostic imaging purposes. As a wave with a frequency (e.g., the acoustic frequency, units of Hz) the ultrasound waves can also be described in terms of period (e.g., the inverse of the frequency, units of time), and wavelength (e.g., the velocity of the wave divided by the frequency, the velocity of the wave multiplied by the period, units of distance such as nm). The velocity of an ultrasound wave is the speed of sound in the relevant medium (e.g., bone, soft tissue, water, air, etc., units of distance per time, m/s). Exemplary equations include the following:

$$f = \frac{1}{T}$$

where f is the acoustic frequency of the ultrasound wave, which may be expressed in Hz; where T is the period of the ultrasound wave, which may be expressed in seconds;

$$λ = \frac{v}{f} \\ λ = v * T$$

where λ is the wavelength of the ultrasound wave, which may be expressed in mm;

Ultrasound can also be characterized by the power of the ultrasound wave, or the total amount of acoustic energy emitted by an ultrasound transducer into the surrounding medium per unit time (e.g., measured in Watts). The power output by an ultrasound transducer can be calculated by the power input to the transducer multiplied by the efficiency of the ultrasound transducer.

$$P output = P input * η$$

where P output is the power output by the ultrasound transducer, which may be expressed in Watts;
where P input is the power input to the ultrasound transducer, which may be expressed in Watts;
where η is the efficiency of the ultrasound transducer, a unitless quantity between 0 and 1;

The power of ultrasound transmitted to tissue can be determined with the following equation, considering the efficiency of the transducer and the acoustic impendence of the tissue and the transmission medium.

$$P transmitted = P input * η * \frac{4 * Z 1 * Z 2}{{( Z 1 + Z 2 )}^{2}}$$

where Z1 is the acoustic impedance of the first medium (e.g., the transducer or the coupling gel), which may be expressed in units of Rayls (a kg/m2/s);
where Z2 is the acoustic impedance of the second medium (e.g., the tissue), which may be expressed in units of Rayls (a kg/m2/s);
where the acoustic impedance Z1 and Z2 is given by

$$Z = ρ * c$$

where ρ is the density of the medium, which may be expressed in units of g/cm3 or g/mL;
where c is the speed of sound in the medium, which may be expressed in units m/s.

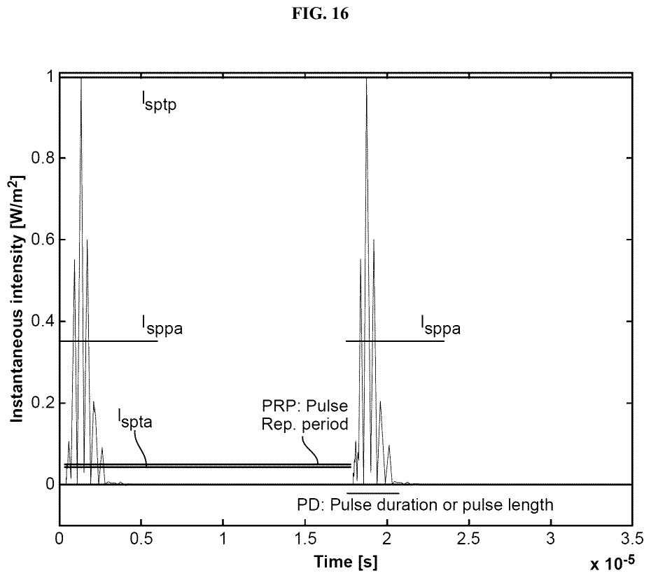

In most diagnostic and therapeutic applications of ultrasound, the ultrasound waves are applied in pulses, more commonly referred to as pulsed ultrasound. Pulsed ultrasound is a type of ultrasound wave emission in which sound energy is transmitted in short, intermittent bursts or pulses rather than as a continuous wave. This pulsing mechanism is commonly used in diagnostic imaging and therapeutic applications because it allows better control of sound wave propagation, timing, interaction with tissues, and will limit the generation of heat and allow for cooling between pulses for some higher power applications. For example, in diagnostic imaging, an image is only created when ultrasound is not being transmitted and is being received the transducer, with the ultrasound transmitted during short pulses. Pulsed ultrasound can be characterized by Pulse Duration (PD), which is the time over which a single ultrasound pulse lasts. With reference to FIG. 16, the pulse duration is labeled as PD and is measured in units of time (e.g., seconds, or microseconds). Pulsed ultrasound can be characterized by Pulse Repetition Period (PRP), which is the time interval between the start of one pulse and the start of the next and is measured in units of time (e.g., seconds, or microseconds). With reference to FIG. 16, the pulse repetition period is labeled as PRP. Pulsed ultrasound can be characterized by Pulse Repetition Frequency (PRF), which is the number of pulses emitted per second (e.g., Hz), and is the inverse of the pulse repetition period. Pulsed ultrasound can be characterized in terms of duty cycle (DC) or duty factor, which is the fraction of time that the ultrasound system is actively transmitting pulses, and is a dimensionless ratio usually expressed as a percentage.

$$Duty Cycle ( DC ) = \frac{PD}{PRP}$$

Ultrasound can further be characterized in terms of ultrasound intensity, which refers to the power of the ultrasound beam per unit area, typically measured in watts per square centimeter (e.g., W/cm², or mW/cm²). Continuous ultrasound (e.g., not pulsed ultrasound) intensity can be characterized in considering the power of the ultrasound beam per unit area. Pulsed ultrasound intensity can be characterized in terms of not only the power of the ultrasound beam per unit area, but also considering the spatial peak or spatial average values of the power applied relative to the point in the application of the pulsed ultrasound, for example: the instantaneous intensity; the temporal peak or the temporal average values; the spatial average and temporal peak; the spatial peak temporal peak; and most commonly the spatial peak temporal average. With reference to FIG. 16, the spatial peak temporal peak is labeled Isptp; the spatial peak pulse average intensity is labeled by Isppa; the spatial peak temporal average is labeled Ispta. As is illustrated by FIG. 16, the values of intensity can shift differ significantly depending on which measurement of intensity is utilized.

Instantaneous intensity as a function of time and position (e.g., r\vec{ }, position as a vector quantity) is given by:

$$I instanteous ( t , {r}^{⇀} ) = \frac{Power {( t , {r}^{⇀} )}^{2}}{z}$$

Spatial peak temporal average intensity is given by:

$$I spta ( t , {r}^{⇀} ) = \frac{1}{T} {\int }_{0}^{T} I instanteous ( t , {r}^{⇀} )$$

Alternatively, if given the spatial average intensity, the spatial peak temporal average intensity can be calculated multiplying the spatial average intensity by the duty cycle, and is given by:

$$I spta = I sa * DC$$

Spatial peak temporal peak intensity is given by:

$$I sptp ( t , {r}^{⇀} ) = max of I instaneneous ( t , {r}^{⇀} )$$

Spatial peak pulsed average intensity is calculated as the average intensity over the pulse duration (see, e.g., FIG. 16 at Isppa).

Spatial average intensity of ultrasound applied to a medium (e.g., a tissue) can also be estimated if provided with the peak negative pressure (PNP) of the ultrasound in the medium, the density of the medium, and the speed of sound in the medium, and is given by:

$$I sa = \frac{{PNP}^{2}}{2 * ρ * c}$$

When applied to tissue, ultrasound can also be characterized in terms of thermal index (TI) to estimate the likelihood of tissue temperature increase due to ultrasound exposure. Thermal index varies significantly depending on tissue type, for example, soft tissue vs bone. Thermal index is given by:

$$TI = \frac{I spta}{I reference}$$

where I reference is a reference intensity that would cause a 1° C. temperature increase under specific conditions. I reference for soft tissue is approximately 100 mW/cm2, for bone is approximately 20 mW/cm2, and for cranial bone is approximately 50 mW/cm2.

When applied to tissue in combination with sonoactive agents, ultrasound can also be characterized in terms of mechanical index (MI) to estimate the likelihood of bioeffects in the tissue resulting from the cavitation of the sonoactive agents. Acoustic cavitation is defined as the growth oscillation and subsequent collapse of air/gas filled structures (e.g., bubbles) under the varying pressure field of an ultrasound wave. Cavitation can further be characterized as stable (non-inertial) or non-stable (inertial), where the bubbles will collapse in the latter scenario producing locally high pressures and temperature elevations. The common equation for mechanical index provided below was derived through a combination of experimental observations and theoretical studies aimed at understanding the conditions under which cavitation occurs during ultrasound exposure, with a goal of providing a simple, quantitative measure of the likelihood of mechanical bioeffects, e.g., inertial cavitation, in tissues exposed to diagnostic ultrasound. Because MI was calculated using empirical observations in experimental studies which measured cavitation thresholds under specific ranges of pressure and frequency, the common equation for mechanical index provided below is generally only considered to be valid for peak negative pressure values of up to about 1 MPa, and acoustic frequencies ranging between 0.1 MHz to 15 MHz, which is sufficient for general conditions employing ultrasound used for clinical imaging.

$$MI = \frac{PNP}{\sqrt{f}}$$

The bioeffects of ultrasound vary significantly with the ultrasound parameters applied and the interaction of the ultrasound energy with the medium through which it propagates. In biological subjects, significant bioeffects include mechanical and thermal effects upon the tissue resulting from ultrasound application. Ultrasound ranges from mild and low intensity ultrasound used for imaging (e.g., B-mode ultrasound of a fetus) to powerful and high intensity ultrasound used for ablation of tissue in surgical applications (e.g., high intensity focused ultrasound, or HIFU). Ultrasound applied with very low intensities, short pulse durations, and low duty cycles are characteristic of B-mode ultrasound commonly used for imaging, while ultrasound applied at high intensity with longer pulse duration, and higher duty cycles are characteristic of ablation grade HIFU.

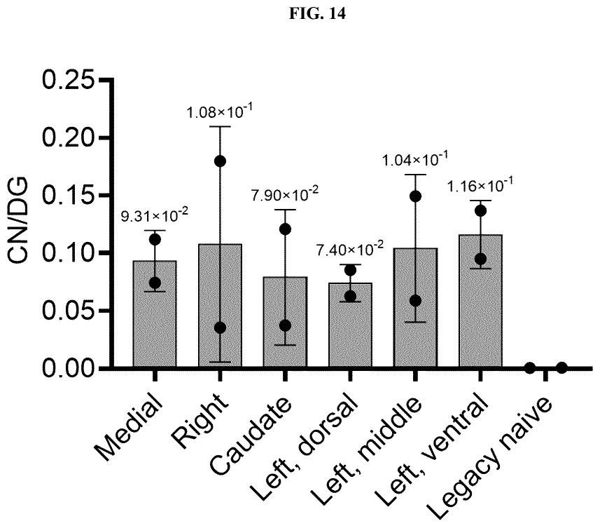

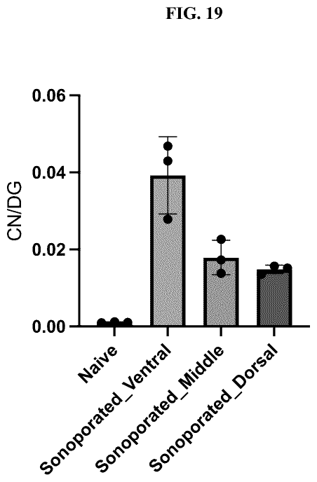

As is shown and described herein, the sonoporation methods of the present disclosure include administering ultrasound to subject tissue, displacing the tissue of the subject with an acoustic radiation force to induce propagation of shear waves throughout the tissue, and further transferring the acoustic pressure field applied from the ultrasound push pulse through the circulatory system to induce a cavitation event of a sonoactive agent, each of which can significantly enhance payload delivery and transfection as compared to standard ultrasound (e.g., B-mode based), without posing the safety risks associated with high-intensity focused ultrasound. When applying ultrasound using standard techniques (e.g., B-mode imaging), the acoustic energy is rapidly dissipated into the subject tissue as distance increases from the source of the ultrasound energy proportional to the square of the distance from the source of the ultrasound energy (in agreement with the inverse square law of energy), leading to poor payload biodistribution and transfection in most organ systems. For example, as is illustrated by Example XIV and FIG. 19, ventral regions of a treated liver closest the source of the ultrasound exhibited significantly higher copy numbers as opposed to middle and dorsal regions of the treated organ. In contrast, the application of ultrasound using the methods disclosed herein displaces a tissue of the subject, inducing shear waves in the tissue and increasing its permeability to exogenous payloads, while efficiently transferring an acoustic pressure field from an ultrasound push pulse through the circulatory system which rapidly induces inertial cavitation of most sonoactive agents present in the circulatory system during transmission of the acoustic pressure field, leading to high payload delivery throughout the treated organ. For example, as is illustrated by Example IV and FIG. 14, subjects treated with the methods disclosed herein exhibit increased copy numbers throughout the treated organ, which are highly uniform even in regions of the organ distal from the ultrasound transducer.

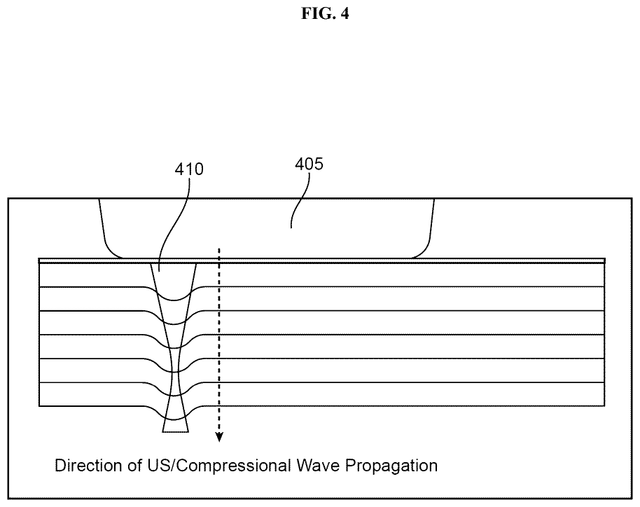

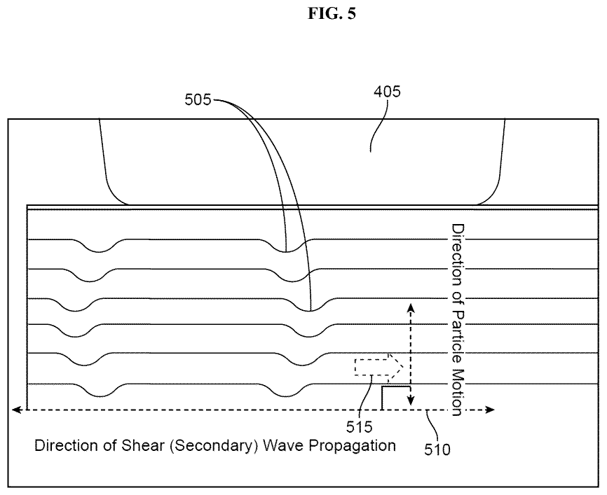

Aspects of the sonoporation methods disclosed herein include applying intermediate intensity ultrasound to apply an acoustic radiation force displacing a tissue of the subject with an acoustic pressure wave and enhancing delivery of an exogenous payload throughout an organ. Application of an acoustic pressure wave displacing a biologic tissue results in mechanical bioeffects which are beneficial to delivery of exogenous payloads. For example, application of an acoustic pressure wave displacing an elastic tissue (which includes most biological tissues aside from bone) can result in the generation of shear waves in the treated tissue. Shear waves, or transverse waves, arise in an elastic medium due to the conservation of momentum and the material's resistance to shear deformation. In such a medium, Newton's second law (F=ma) governs the relationship between forces and motion, where any applied force results in an acceleration of material particles. For example, with reference to FIGS. 4-5 when a tangential acoustic radiation force (FIG. 4) is applied, it induces shear stress in the treated tissue, which leads to shear strain and particle displacement (FIG. 5) perpendicular to the direction of the applied force in the treated tissue. As the tissue resists deformation from the application of the acoustic radiation force, the elasticity of the tissue provides a restoring force which tends to return the displaced portions of the tissue to its original position. As the acoustic radiation force propagates to neighboring particles in the connected tissue, a wave like motion occurs in the tissue and moves throughout the treated organ as the displaced tissue returns to its initial position.

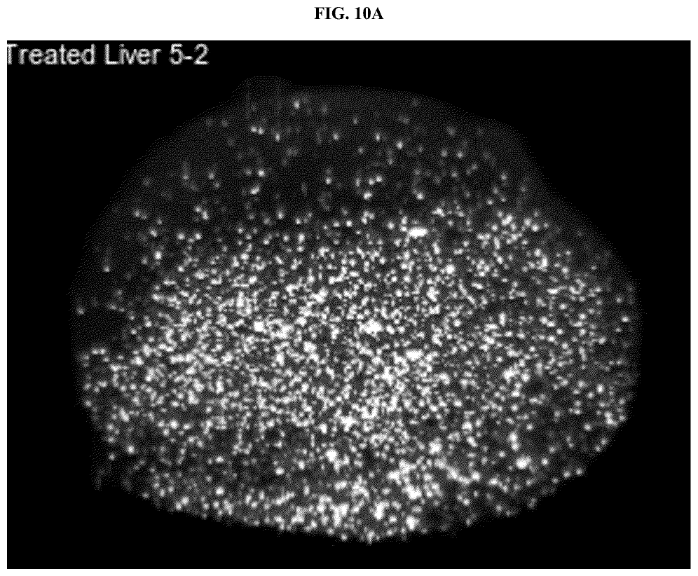

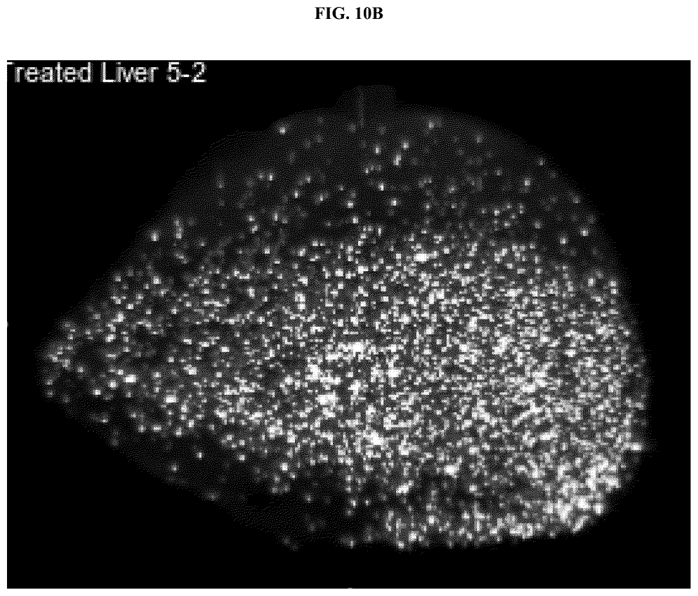

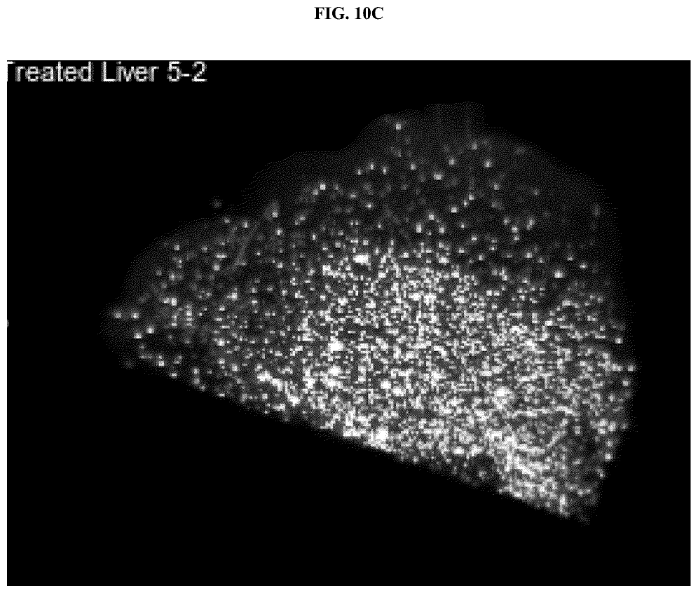

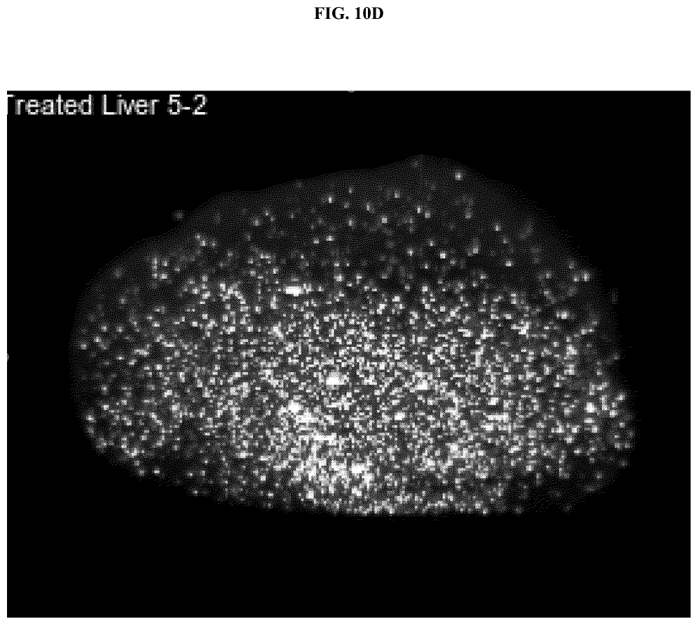

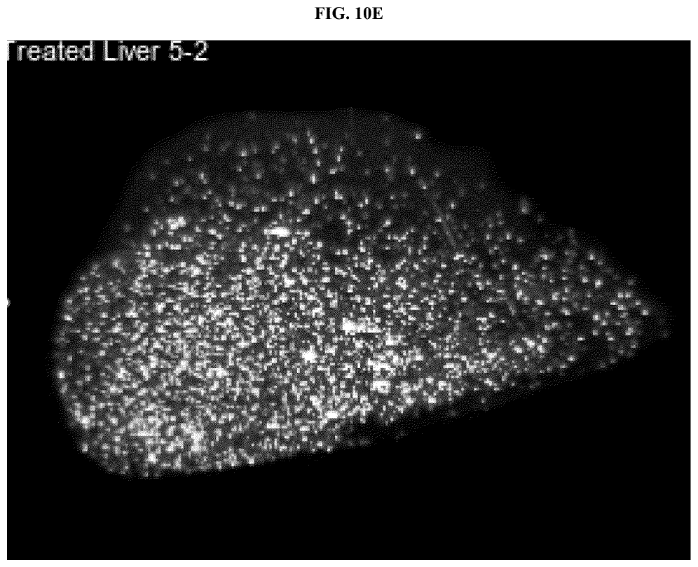

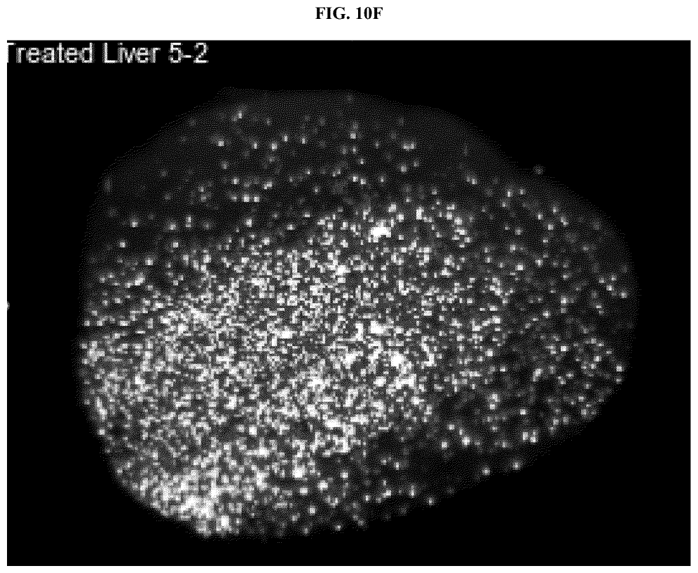

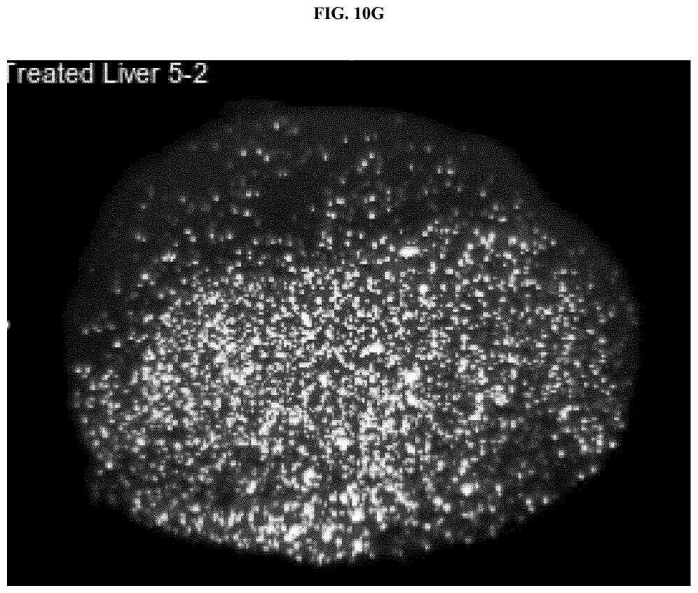

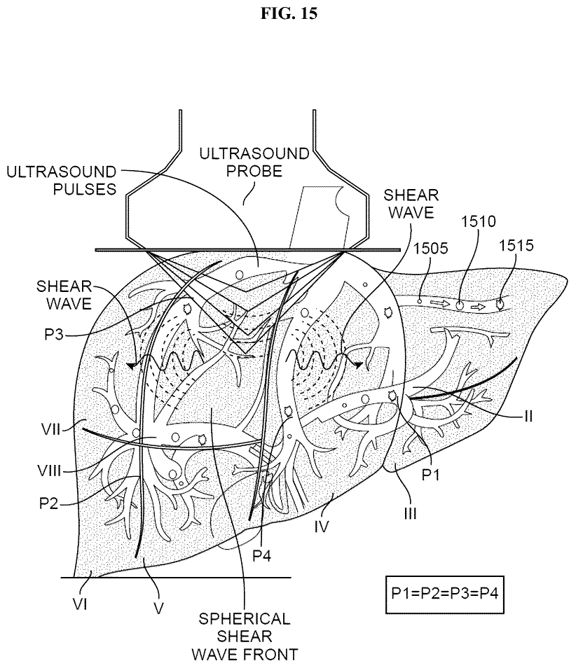

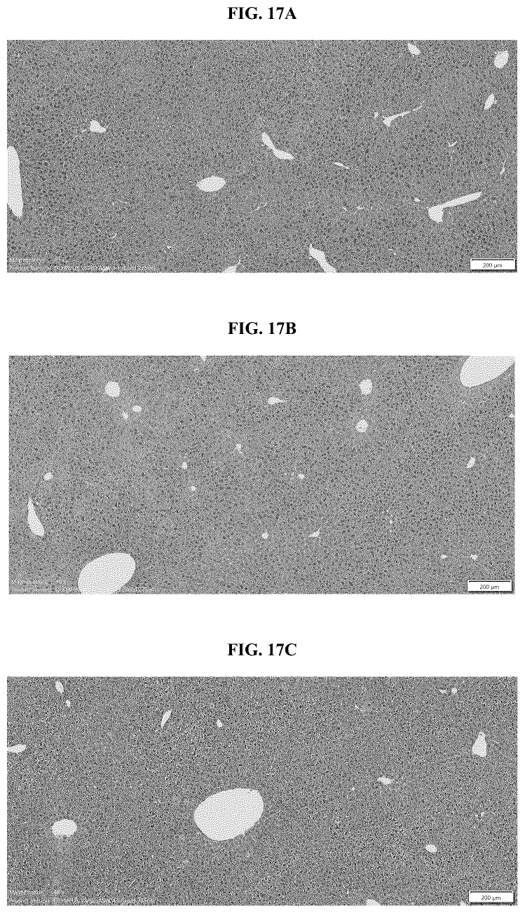

Without being bound to a particular theory, as the treated tissue is displaced by the ultrasound, shear waves propagate through the organ, with the movement of the tissue regions inducing a localized pressure gradient in the tissue and a resulting mechanical stress. The mechanical stress within the tissue induced by the propagation of shear waves can disrupt tight junctions in the epithelial and endothelial tissues of the treated organ, increasing the permeability of the tissues to an exogenous payload. For instance, the mechanical stress in the tissue resulting from the propagation of shear waves in the tissue can exert a force upon the tight junction complex and lead to a temporary opening of the tight junction complex by disruption interactions between junctional adhesion proteins, deformation of cytoskeletal structures due to altering the tension and alignment of actin filaments, temporarily increasing paracellular permeability. In addition, the application of an acoustic radiation force with propagation of the shear waves through the organ can also transiently increase the volume of the interstitial space between cells, providing greater opportunity for entry of payloads into cells from interstitial fluid. Further, the propagation of shear waves through the tissue results in pressure variations in distal regions of the tissue outside of the ultrasound axial beam, and will induce cavitation events of sonoactive agents in such regions distal regions. Application of an acoustic radiation force resulting in the propagation of shear waves through a treated tissue with the ultrasound parameters disclosed herein (for example, ultrasound intensity, pulse length and duty cycle) provides a beneficial technical effect of increasing the delivery and biodistribution (and transfection where applicable) of the payload throughout the treated organ without adverse effects on the treated tissue (see, for example, FIGS. 10A-10G showing increased delivery and biodistribution, and FIG. 17A-17C showing no adverse effects on treated tissue). In this manner propagation of the shear waves spherically throughout the non-vascularized regions of the treated organ increases transfection efficiency even within regions of the treated organ distal from the ultrasound transducer not necessarily within the axial beam of the ultrasound (see, for example, Example IV and FIG. 14 showing similar copy numbers in the treated organ even in regions distal from the ultrasound transducer; compare with Example XIV and FIG. 19 showing a rapid loss of delivery efficiency as distance increases from an ultrasound transducer), representing a surprising and unexpected result in the increased efficiency and improved biodistribution of the payload. With reference to FIG. 15, ultrasound pulses applying an acoustic radiation force to a ventral region of the liver can induce shear waves in the non-vascularized regions III, IV, V, VI, VIII of the tissue which will propagate through the organ and improve transfection in such regions III, IV, V, VI of the organ distal from the source of the ultrasound. In some cases, inducing propagation of shear waves throughout tissue in a treated organ by application of an acoustic radiation force at an ultrasound intensity (Ispta) of 100-5000 mW/cm2 provides a beneficial technical effect of increasing the delivery and biodistribution (and transfection where applicable) of the payload throughout the target organ without adverse effects on the treated tissue. In some cases, inducing propagation of shear waves throughout tissue in a treated organ by application of an acoustic radiation force at an ultrasound intensity (Ispta) of at least 100 mW/cm2 and a pulse length of at least 20 μs provides a beneficial technical effect of increasing the delivery and biodistribution (and transfection where applicable) of the payload throughout the target organ without adverse effects on the treated tissue. In some cases, inducing propagation of shear waves throughout tissue in a treated organ by application of an acoustic radiation force at a duty cycle of less than 5% and a pulse length of greater than 200 μs provides a beneficial technical effect of increasing the delivery and biodistribution (and transfection where applicable) of the payload throughout the target organ without adverse effects on the treated tissue. In some cases, inducing propagation of shear waves throughout tissue in a treated organ by application of an acoustic radiation force at a pulse length of greater than 200 μs (microseconds) and at an ultrasound intensity (Ispta) of up to 5000 mW/cm2 provides a beneficial technical effect of increasing the delivery and biodistribution (and transfection where applicable) of the payload throughout the target organ without adverse effects on the treated tissue. In some cases, inducing propagation of shear waves throughout tissue in a treated organ by application of an acoustic radiation force at a pulse length of greater than 200 μs (microseconds) and at an ultrasound intensity of at least 100 mW/cm2 and duty cycle of less than 1% provides a beneficial technical effect of increasing the delivery and biodistribution (and transfection where applicable) of the payload throughout the target organ without adverse effects on the treated tissue. In some cases, increasing the delivery of an exogenous payload comprising a nucleic acid results in delivery of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some cases, increasing the delivery of an exogenous payload comprising a nucleic acid which is DNA results in delivery of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some embodiments, the increased delivery and biodistribution results in increased expression of a nucleic acid payload. In some embodiments, the payload is delivered and expressed throughout the organ of the nucleic acid payload in every lobe of the organ. In some embodiments, the expression of the nucleic acid payload throughout the organ comprises inducing said expression of the nucleic acid payload in two samples of the organ taken from opposite ends of the organ. In some embodiments, the delivery of the nucleic acid payload throughout the organ comprises delivering of the nucleic acid payload in two samples of the organ taken from opposite ends of the organ. In some embodiments, the samples are samples sized up to 1 cm³ or up to 1 g. In some embodiments, the method delivers at least 0.005 copies per diploid genome of the payload to the tissue. In some embodiments, the method delivers at least at least 0.01 copies per diploid genome of the payload to the tissue.

In some embodiments, the expression of the nucleic acid payload is at least 0.005 copies per diploid genome. In some embodiments, the expression of the nucleic acid payload is at least 0.01 copies per diploid genome.

In some embodiments, inducing propagation of shear waves throughout tissue in a treated organ and the beneficial technical effect of increasing the delivery and biodistribution (and transfection where applicable) of the payload throughout the target organ without adverse effects on the treated tissue can be achieved by the applying ultrasonic acoustic energy to the tissue at an ultrasound intensity of: at least 100, 150, 175, 200, 225, 250, 275, 300, 350, 400, 450, 500, 600, 700, 800, 900, 1000, 1250, 1500, 1750, 2000, 2250, 2500, 2750, 3000, 3250, 3500, 3750, 4000, 4250, 4500, or 4750 mW/cm2. In some embodiments, a beneficial technical effect of increasing the delivery and biodistribution (and transfection where applicable) of the payload throughout the target organ without adverse effects on the treated tissue can be achieved by the applying ultrasonic acoustic energy to the tissue at an ultrasound intensity of up to 500, 600, 700, 800, 900, 1000, 1250, 1500, 1750, 2000, 2250, 2500, 2750, 3000, 3250, 3500, 3750, 4000, 4250, 4500, 4750, or 5000 mW/cm2.

In some embodiments, inducing propagation of shear waves throughout tissue in a treated organ and the beneficial technical effect of increasing the delivery and biodistribution (and transfection where applicable) of the payload throughout the target organ without adverse effects on the treated tissue can be achieved by the applying ultrasonic acoustic energy to the tissue at an ultrasound pulse length of at least 20 us; a pulse length is about 100 microseconds to about 500 microseconds; a pulse length 200-5000 us; a pulse length of at least 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us; or a pulse length of up to 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us.

In some embodiments, inducing propagation of shear waves throughout tissue in a treated organ and the beneficial technical effect of increasing the delivery and biodistribution (and transfection where applicable) of the payload throughout the target organ without adverse effects on the treated tissue can be achieved by the applying ultrasonic acoustic energy to the tissue at a duty cycle of up to 5, 4, 3, 2, or 1%, at a duty cycle of up to 0.1, at a duty cycle of 0.01%-1.0%. In some embodiments, the applying ultrasonic acoustic energy to the tissue comprises applying the ultrasound acoustic energy at duty cycle of at least 0.05% and less than 2.0%.

In addition to inducing propagation of shear waves throughout tissue in a treated organ by application of an acoustic radiation force, application of the ARF also promotes additional cavitation events within tissue vasculature. Without being bound to a particular theory, application of an acoustic radiation force can transmit an acoustic pressure field throughout the vasculature of the organ because of the incompressibility of blood within the organ. If modeling the change in pressure in the organ using a quasi-static approximation of Pascal's law, assuming the subject's circulatory system can be considered an enclosed system with a confined, incompressible and slowly moving fluid (e.g., blood) therein having a slow response time of blood to move in response to a shift in pressure; the change in pressure resulting from the acoustic radiation force will be transmitted through proximal vessels to distal vessels of the organ remote, increasing cavitation events therein. With reference to FIG. 15, the acoustic radiation force can induce cavitation of the sonoactive agent 1505/1510/1515 causing it to rapidly expand 1510 and contract 1505 in vibrational cavitation, eventually leading to inertial cavitation of the sonoactive agent 1515 as the bubble collapses. With reference to FIG. 15, in applying an acoustic radiation force to the liver, a pressure field will be transferred through blood, and the pressure at a point P3 (near the ultrasound transducer) will be substantially the same as the pressure at other points (P1, P2, P4) in connected major arteries or veins II, VIII of the treated organ. In the presence of a sonoactive agent which undergoes cavitation in a liquid medium (e.g., blood) when exposed to an acoustic pressure field, an acoustic pressure field of sufficient magnitude applied with ultrasound parameters as disclosed herein will be efficiently transmitted throughout the vasculature of, and can more rapidly and more uniformly induce cavitation of the sonoactive agents 1505/1510/1515 throughout the vasculature of the treated organ as compared to standard ultrasound (e.g., B-mode), including in distal regions of the organ remote from the ultrasound transducer, further increasing payload delivery and transfection. Moreover, the efficiency of delivery and transfection resulting such cavitation events improves as the increased intensity of the ultrasound applying the ARF moves the sonoactive agent towards the vessel walls prior to inducing a cavitation event due to acoustic streaming effects.

In some cases, the application of an acoustic radiation force induces cavitation of a sonoactive agent in tissue throughout the treated organ. In some cases, the application of an acoustic radiation force induces stable vibrational cavitation of a sonoactive agent in tissue throughout the treated organ. In some cases, the application of an acoustic radiation force induces inertial cavitation of a sonoactive agent in tissue throughout the treated organ, thereby increasing delivery of the payload. In some cases, the application of an acoustic radiation force induces inertial cavitation of a sonoactive agent in substantially all regions of the treated organ, thereby increasing payload delivery and transfection. In some cases, the application of an acoustic radiation force transmits an acoustic pressure field which induces inertial cavitation of a sonoactive agent throughout the vasculature of the treated organ, thereby increasing payload delivery and transfection. For example, when applying an acoustic radiation force of sufficient intensity as disclosed herein to a vascularized organ, almost all of the sonoactive agents in circulation in the organ can be disrupted by initial cavitation simultaneously due to the highly efficiency transmission of pressure throughout the circulatory system, increasing the permeability of the treated tissue to the exogenous payload throughout the entire organ (see, for example, Example IV and FIG. 14 showing uniform copy numbers in the treated organ even in regions distal from the ultrasound transducer; compare with Example XIV and FIG. 19 showing a rapid loss of delivery efficiency as distance increases from an ultrasound transducer). In some cases, the application of an acoustic radiation force with the ultrasound parameters disclosed herein (for example, ultrasound intensity, pulse length and duty cycle) and resulting cavitation of the sonoactive agent throughout the treated organ increases the delivery and biodistribution of the payload throughout the target organ without adverse effects on the treated tissue (see, for example, FIGS. 10A-10G showing increased delivery and biodistribution, and FIG. 17A-17C showing no adverse effects on treated tissue). In some cases, application of an acoustic radiation force with ultrasound acoustic energy at an ultrasound intensity (Ispta) of 100-5000 mW/cm2 induces inertial cavitation of a sonoactive agent throughout the treated organ and provides a beneficial technical effect of increasing the delivery and biodistribution (and transfection where applicable) of the payload throughout the target organ without adverse effects on the treated tissue. In some cases, application of the acoustic radiation force with ultrasound acoustic energy at an ultrasound intensity (Ispta) of at least 100 mW/cm2 and a pulse length of at least 20 μs induces inertial cavitation of a sonoactive agent throughout the treated organ and provides a beneficial technical effect of increasing the delivery and biodistribution (and transfection where applicable) of the payload throughout the target organ without adverse effects on the treated tissue. In some cases, application of the acoustic radiation force with ultrasound acoustic energy at a duty cycle of less than 5% and a pulse length of greater than 200 μs induces inertial cavitation of a sonoactive agent throughout the treated organ and provides a beneficial technical effect of increasing the delivery and biodistribution (and transfection where applicable) of the payload throughout the target organ without adverse effects on the treated tissue. In some cases, application of the acoustic radiation force with ultrasound acoustic energy at a pulse length of greater than 200 μs (microseconds) and at an ultrasound intensity of up to 5000 mW/cm2 induces inertial cavitation of a sonoactive agent throughout the treated organ and provides a beneficial technical effect of increasing the delivery and biodistribution (and transfection where applicable) of the payload throughout the target organ without adverse effects on the treated tissue. In some cases, application of the acoustic radiation force with ultrasound acoustic energy at a pulse length of greater than 200 μs (microseconds) and at an ultrasound intensity of at least 100 mW/cm2 and duty cycle of less than 1%. Induces inertial cavitation of a sonoactive agent throughout the treated organ and increasing the delivery and biodistribution (and transfection where applicable) of the payload throughout the treated organ without adverse effects on the treated tissue. In some embodiments, the increased delivery and biodistribution results in increased expression of a nucleic acid payload. In some cases, increasing the delivery of an exogenous payload comprising a nucleic acid results in delivery of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some cases, increasing the delivery of an exogenous payload comprising a nucleic acid which is DNA results in delivery of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some embodiments, the increased delivery and biodistribution results in increased expression of a nucleic acid payload. In some embodiments, the payload is delivered and expressed throughout the organ of the nucleic acid payload in every lobe of the organ. In some embodiments, the expression of the nucleic acid payload throughout the organ comprises inducing said expression of the nucleic acid payload in two samples of the organ taken from opposite ends of the organ. In some embodiments, the delivery of the nucleic acid payload throughout the organ comprises delivering of the nucleic acid payload in two samples of the organ taken from opposite ends of the organ. In some embodiments, the samples are samples sized up to 1 cm³ or up to 1 g. In some embodiments, the method delivers at least 0.005 copies per diploid genome of the payload to the tissue. In some embodiments, the method delivers at least at least 0.01 copies per diploid genome of the payload to the tissue.

In some embodiments, inertial cavitation of a sonoactive agent throughout the treated organ and associated beneficial technical effect of increasing the delivery and biodistribution (and transfection where applicable) of the payload throughout the target organ without adverse effects on the treated tissue can be achieved by the applying ultrasonic acoustic energy to the tissue at an ultrasound intensity of: at least 100, 150, 175, 200, 225, 250, 275, 300, 350, 400, 450, 500, 600, 700,800, 900, 1000, 1250, 1500, 1750, 2000, 2250, 2500, 2750, 3000, 3250, 3500, 3750, 4000, 4250, 4500, or 4750 mW/cm2. In some embodiments, a beneficial technical effect of increasing the delivery and biodistribution (and transfection where applicable) of the payload throughout the target organ without adverse effects on the treated tissue can be achieved by the applying ultrasonic acoustic energy to the tissue at an ultrasound intensity of up to 500, 600, 700,800, 900, 1000, 1250, 1500, 1750, 2000, 2250, 2500, 2750, 3000, 3250, 3500, 3750, 4000, 4250, 4500, 4750, or 5000 mW/cm2.

In some embodiments, inertial cavitation of a sonoactive agent throughout the treated organ and associated the beneficial technical effect of increasing the delivery and biodistribution (and transfection where applicable) of the payload throughout the target organ without adverse effects on the treated tissue can be achieved by the applying ultrasonic acoustic energy to the tissue at an ultrasound pulse length of at least 20 us, a pulse length is about 100 microseconds to about 500 microseconds; the ARF is applied at an ultrasound pulse length 200-5000 us; the ARF is applied at an ultrasound pulse length of at least 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us; the ARF is applied at an ultrasound pulse length of up to 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us.

In some embodiments, inertial cavitation of a sonoactive agent throughout the treated organ and associated the beneficial technical effect of increasing the delivery and biodistribution (and transfection where applicable) of the payload throughout the target organ without adverse effects on the treated tissue can be achieved by the applying ultrasonic acoustic energy to the tissue at a duty cycle of up to 5, 4, 3, 2, or 1%, at a duty cycle of up to 0.1, at a duty cycle of 0.01%-1.0%. In some embodiments, the applying ultrasonic acoustic energy to the tissue comprises applying the ultrasound acoustic energy at duty cycle of at least 0.05% and less than 2.0%. In some embodiments, the acoustic radiation force is applied for at least 1, 5, or 10. In some embodiments, the acoustic radiation force is applied for up to 30, 45, 60, 75, or 90 minutes.

In some cases, the ultrasound parameters disclosed herein can induce both propagation of shear waves throughout tissue as well as uniform disruption of sonoactive agents throughout a treated organ, further enhancing delivery of a payload throughout the target organ without adverse effects on the treated tissue. In some cases, the nearly simultaneously disruption of substantially all of the sonoactive agents in circulation in the organ by initial cavitation occurs upon application of an acoustic radiation force of sufficient intensity, with the cavitation of the sonoactive agent creating pores in endothelial tissue and cell membranes throughout the organ, followed by the acoustic radiation force propagating of shear waves through the treated tissue and increasing the permeability of the tissue due to the localized pressure gradient in the tissue and resulting mechanical stress from the tissue displacement. In some cases, the propagation of shear waves through the tissue results in pressure variations in distal regions of the tissue outside of the ultrasound axial beam, and will induce cavitation events of sonoactive agents in such regions distal regions, further increasing payload delivery. In some cases, the application of an acoustic radiation force at an ultrasound intensity (Ispta) of 100-5000 mW/cm2 induces propagation of shear waves in the tissue and simultaneously disrupts most or all of the sonoactive agents in circulation in the treated organ, providing a technical effect of increasing the delivery and biodistribution (and transfection where applicable) of the payload throughout the target organ without adverse effects on the treated tissue. In some cases, the application of an acoustic radiation force at an ultrasound intensity (Ispta) of 100-5000 mW/cm2 induces propagation of shear waves in the tissue and simultaneously disrupts most or all of the sonoactive agents in circulation in the treated organ, providing a technical effect of increasing the delivery and biodistribution (and transfection where applicable) of the payload throughout the target organ without adverse effects on the treated tissue. In some cases, the application of an acoustic radiation force at a pulse length of greater than 200 μs (microseconds) and at an ultrasound intensity (Ispta) of up to 5000 mW/cm2 induces propagation of shear waves in the tissue and simultaneously disrupts most or all of the sonoactive agents in circulation in the treated organ, providing a technical effect of increasing the delivery and biodistribution (and transfection where applicable) of the payload throughout the target organ without adverse effects on the treated tissue. In some cases, the application of an acoustic radiation force at a duty cycle of less than 5% and a pulse length of greater than 200 us (microseconds) induces propagation of shear waves in the tissue and simultaneously disrupts most or all of the sonoactive agents in circulation in the treated organ, providing a technical effect of increasing the delivery and biodistribution (and transfection where applicable) of the payload throughout the target organ without adverse effects on the treated tissue. In some cases, the application of at an ultrasound intensity (Ispta) of at least 100 mW/cm2 and duty cycle of less than 1% induces propagation of shear waves in the tissue and simultaneously disrupts most or all of the sonoactive agents in circulation in the treated organ, providing a technical effect of increasing the delivery and biodistribution (and transfection where applicable) of the payload throughout the target organ without adverse effects on the treated tissue.

Aspects disclosed herein provide methods disclosed herein include applying intermediate intensity ultrasound to apply an acoustic radiation force displacing a tissue of the subject with an acoustic pressure wave and enhancing delivery of an exogenous payload throughout an organ. As is described herein, the present disclose provides for techniques which can induce both propagation of shear waves throughout tissue as well as uniform disruption of sonoactive agents throughout a treated organ. However, in addition inducing uniform cavitation of sonoactive agents throughout an organ and inducing propagation of shear waves to increase payload delivery, the techniques and ultrasound parameters disclosed herein also do not result in significant thermal effects (e.g., heating) in the target tissue, tissue toxicity, histological changes, cell death, or other tissue damage following the procedure, thereby providing a highly tolerable procedure which can be repeatedly administered to a subject. In some cases, the methods disclosed herein do not result in significant heating of the treated tissue. In some cases, the application of the ARF does not increase the temperature of the tissue by more than 1 C. In some cases, the application of the ARF does not increase the temperature of the tissue by more than 0.5 C. In some cases, the application of the ARF does not increase the temperature of the tissue by more than 0.25 C. In some cases, the application of the ARF does not increase the temperature of the tissue by more than 0.1 C. In some cases, a second sonoporation treatment is applied to a subject using the methods disclosed herein, in which a second dose of the exogenous payload and a second dose of the sonoactive agent are administered to the subject and a second acoustic radiation force (ARF) is applied to the organ of the subject. In some cases, a third sonoporation treatment is applied to a subject using the method disclosed herein, in which a third dose of the exogenous payload and a third dose of the sonoactive agent are administered to the subject and a third application of an acoustic radiation force (ARF) is applied to the organ of the subject. In some cases, application of an acoustic radiation force with ultrasound acoustic energy at an ultrasound intensity (Ispta) of 100-5000 mW/cm2 does not substantially increase the temperature of the treated tissue, and the acoustic radiation force can be reapplied in a second or a third treatment session without adverse impacts to the treated tissue. In some cases, application of an acoustic radiation force with ultrasound acoustic energy at a pulse length of greater than 200 μs (microseconds) and at an ultrasound intensity (Ispta) of up to 5000 mW/cm2 does not substantially increase the temperature of the treated tissue, and the acoustic radiation force can be reapplied in a second or a third treatment session without adverse impacts to the treated tissue. In some cases, application of an acoustic radiation force with ultrasound acoustic energy at a duty cycle of less than 5% and a pulse length of greater than 200 us (microseconds) does not substantially increase the temperature of the treated tissue, and the acoustic radiation force can be reapplied in a second or a third treatment session without adverse impacts to the treated tissue. In some cases, application of an acoustic radiation force with ultrasound acoustic energy at an ultrasound intensity (Ispta) of at least 100 mW/cm2 and duty cycle of less than 1% does not substantially increase the temperature of the treated tissue, and the acoustic radiation force can be reapplied in a second or a third treatment session without adverse impacts to the treated tissue. Adverse impacts to treated tissue can include significant thermal effects (e.g., heating) in the target tissue, tissue toxicity, histological changes, cell death, or other tissue damage. In some cases, the methods of distributing a payload across an organ with application of an acoustic radiation force as disclosed herein generates expression of at least 1 copy per nanogram of subject DNA throughout the treated organ. In some cases, the method of distributing a payload across an organ with application of an acoustic radiation force as disclosed herein generates expression of at least 5, 10, 15, 20, or 25 copies per nanogram of subject DNA throughout the treated organ. In some cases, reapplication of the acoustic radiation force in a second or a third treatment session significantly increases the delivery of the payload to the treated tissue. In some cases, reapplication of the acoustic radiation force in a second or a third treatment session significantly increases the expression of the payload throughout the treated organ. In some embodiments, the acoustic radiation force is applied for at least 1, 5, or 10. In some embodiments, the acoustic radiation force is applied for up to 30, 45, 60, 75, or 90 minutes.

Sonoporation refers to the delivery of therapeutic agents, for example nucleic acids, using ultrasound and/or sonoactive agents (e.g., sonoactive microstructures). Disclosed herein are methods of sonoporation in which an exogenous payload is delivered to a cell in a tissue of a subject using an acoustic radiation force applied using ultrasound. Aspects of the sonoporation methods disclosed herein may also include inducing displacing the tissue of the subject with the acoustic radiation force to induce propagation of shear waves throughout the tissue of the subject thereby enhancing delivery of a nucleic acid payload to a cell. As shown and described herein, methods of sonoporation in which using acoustic radiation force to induce propagation of shear waves in a tissue of a subject can significantly enhance payload delivery and/or gene expression as compared to traditional ultrasound techniques. The method may further include applying the acoustic radiation force to induce propagation of shear waves throughout the tissue in combination with other secondary ultrasound energies such as plane wave ultrasound or focused beam ultrasound in which the secondary ultrasound energy moves sonoactive microstructures endothelial border of a tissue comprising the cell, while applying the acoustic radiation force during shear wave propagation induces inertial cavitation of sonoactive microstructures at the endothelial border of the tissue comprising the cell, thereby enhancing delivery of the therapeutic payload to a cell, and, in cases of a nucleic acid payload, resulting gene expression.

Aspects disclosed herein provide a method of delivering an exogenous payload to a cell in a tissue of a subject, the method comprising: delivering an exogenous payload to the subject; and applying an acoustic radiation force (ARF) to the subject, thereby generating shear waves in the tissue of the subject, wherein the acoustic radiation force enhances delivery of the exogenous payload to the cell in the tissue of an organ of the subject. Aspects disclosed herein provide a method of distributing a payload across an organ, comprising: administering the payload and a sonoactive agent to the subject, and applying an acoustic radiation force (ARF) sufficient to deliver at least 1 copy per nanogram throughout the organ. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject displacing the tissue by at least 0.001 mm, thereby generating shear waves in the tissue of the subject, wherein the acoustic radiation force enhances delivery of the exogenous payload to the cell in the tissue of an organ of the subject. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy having a pulse length of greater than 200 μs (microseconds) and induces a peak negative pressure of at least 3,000 kPa in the tissue. Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at an ultrasound intensity of at least 100 mW/cm2 and a pulse length of greater than 20 μs (microseconds). Aspects disclosed herein provide a method of delivering an exogenous payload to a cell in a tissue of a subject, the method comprising: delivering an exogenous payload to the subject; administering a sonoactive agent to the subject; and applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at an ultrasound intensity of at least 100 mW/cm2 and duty cycle of less than 1%. In some embodiments, the shear waves induced by application of the ARF improve distribution of the payload throughout the organ, and provide a beneficial technical effect of improving payload delivery and resulting gene expression. In some cases, applying the ARF with an ultrasound having a pulse length of greater than 200 μs (microseconds), a duty cycle of less than 1%, and/or an intensity of at least 100 mW/cm2 can improve distribution of the payload throughout the organ, and provide a beneficial technical effect of improving payload delivery and resulting gene expression.

In some embodiments the payload is an exogenous payload, and wherein the ARF generates shear waves in a tissue of the organ of the subject. In some embodiments the payload is expressed in the cell at an expression level of at least 1.25, 1.5, 1.75, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some embodiments the payload is expressed throughout the organ in every lobe of the organ. In some embodiments the payload is expressed throughout the organ in two samples of the organ taken from opposite ends of the organ. In some embodiments the samples are samples sized up to 1 cm³ or up to 1 g. In some embodiments the payload is expressed in the cell at an expression level of at least 0.005 copies per diploid genome. In some embodiments the payload is expressed in the cell at an expression level of at least 0.01 copies per diploid genome. In some embodiments the ARF displaces the tissue of the subject displacing the tissue by at least 0.001 mm. In some embodiments the ARF is applied using ultrasound acoustic energy having a pulse length of greater than 200 μs (microseconds) and induces a peak negative pressure of at least 3,000 kPa in the tissue. In some embodiments the ARF is applied using ultrasound acoustic energy at an ultrasound intensity of at least 100 mW/cm2 and a pulse length of greater than 20 μs (microseconds). In some embodiments the ARF is applied using ultrasound acoustic energy at an ultrasound intensity of at least 100 mW/cm2 and a duty cycle of less than 1%. In some embodiments the ARF is applied using ultrasound acoustic energy having a pulse length of greater than 200 μs (microseconds) and induces a peak negative pressure of at least 3300, 3500, 4000, 4500, 5000, 5500, 6000, 6500, 7000, or 7500 kPa in the tissue. In some embodiments the ARF is applied at a thermal index of less than 1.0. In some embodiments the ARF is applied at a thermal index of 0.01-1.0. In some embodiments the ARF is applied at a thermal index of 0.1-1.0. In some embodiments the application of the ARF does not increase the temperate of the tissue by more than 1 C. In some embodiments the application of the ARF does not increase the temperate of the tissue by more than 0.5 C. In some embodiments the application of the ARF does not increase the temperate of the tissue by more than 0.1 C.

In some embodiments, the method includes administering a plurality of sonoactive microstructures to the subject. In some embodiments, the acoustic radiation force is applied using an ultrasound probe applying ultrasound acoustic energy to the tissue. In some embodiments, the ultrasound probe comprises a plurality of piezoelectric elements configured to emit ultrasound acoustic energy. In some embodiments, portions of the plurality of piezoelectric elements are arranged in one or more arrays. In some embodiments, the ultrasound probe is a phased array transducer comprising a plurality of piezoelectric elements configured to emit ultrasound acoustic energy. In some embodiments, the ultrasound probe is a phased array ultrasound probe, a linear ultrasound probe, a curvilinear ultrasound probe, a convex array ultrasound probe, an endocavitary ultrasound probe, a 3D ultrasound probe, a 4D ultrasound probe, a Doppler ultrasound probe, or a color doppler ultrasound probe. In some embodiments, separate portions of the plurality of the piezoelectric elements each emit an ultrasound beam, wherein the acoustic radiation force (ARF) is applied using a plurality of ultrasound beams. In some embodiments, separate arrays each emit an ultrasound beam, wherein the acoustic radiation force (ARF) is applied using a plurality of ultrasound beams. In some embodiments, the ARF is a focused acoustic radiation force and the focused acoustic radiation force is applied using a plurality of ultrasound beams. In some embodiments, the plurality of ultrasound beams produce a plurality of shear waves in the tissue, wherein at least two of the plurality of shear waves each originate at a different location in the tissue. In some embodiments, a first shear wave of the plurality of shear waves in the tissue constructively interferes with a second shear wave of the plurality of shear waves in the tissue. In some embodiments, applying the acoustic radiation force induces inertial cavitation of a portion of the plurality of sonoactive microstructures. In some embodiments, the ARF induces a compressional wave in the tissue. In some embodiments the compressional wave is followed by a rarefaction wave which is a negative acoustic force in the tissue. In some embodiments, the compressional wave induces a rarefaction wave in the tissue. In some embodiments, the rarefaction wave in the tissue induces inertial cavitation of the microbubbles. In some embodiments, inducing inertial cavitation of a portion of the plurality of sonoactive microstructures during propagation of the shear wave in the tissue increases delivery of the exogenous payload to the cell. In some embodiments, the plurality of sonoactive microstructures comprise a protein-stabilized microstructure. In some embodiments, the plurality of sonoactive microstructures comprise a phospholipid stabilized microstructure. In some embodiments, the plurality of sonoactive microstructures are non-phase-shiftable microstructures.

In some embodiments, the acoustic radiation force is applied using an ultrasound probe applying ultrasound acoustic energy to the tissue. In some embodiments, the ARF displaces the tissue of the subject. In some embodiments, the shear waves displace the tissue of the subject. In some embodiments, a tissue displacement is at least 0.001 mm. In some embodiments, a tissue displacement ranges from at least 0.001 mm to about 5 mm. In some embodiments, a tissue displacement ranges from 0.01 mm to about 1 mm. In some embodiments, a displacement of the shear waves in the tissue is by at least 0.001 mm. In some embodiments, a displacement of the shear waves in the tissue is by at least 0.01 mm. In some embodiments, a displacement of the shear waves in the tissue is by 0.01-1 mm.

In some embodiments, the acoustic radiation force is applied using an ultrasound probe applying ultrasound acoustic energy to the tissue. In some embodiments, the ultrasonic acoustic energy is applied at a mechanical index of greater than 0.4. In some embodiments, the ultrasonic acoustic energy is applied at a mechanical index of about 1.4. In some embodiments, the ultrasonic acoustic energy is applied at a mechanical index of at least 1.3. In some embodiments, the ultrasonic acoustic energy is applied at a mechanical index of greater than 0.4 up to about 3.0. In some embodiments, the ultrasonic acoustic energy is applied at a frequency of about 0.1 MHz to about 10 MHz. In some embodiments, the ultrasonic acoustic energy is applied at a frequency of about 2.5 MHz. In some embodiments, the applying ultrasonic acoustic energy to the tissue comprises applying the ultrasound acoustic energy at an ultrasound intensity of at least 100 mW/cm². In some embodiments, the applying ultrasonic acoustic energy to the tissue comprises applying the ultrasound acoustic energy at an ultrasound intensity of about 100 mW/cm² to about 10,000 mW/cm². In some embodiments, the applying ultrasonic acoustic energy to the tissue comprises applying the ultrasound acoustic energy at an ultrasound intensity of about 100 mW/cm² to about 5,000 mW/cm². In some embodiments, the applying ultrasonic acoustic energy to the tissue comprises applying the ultrasound acoustic energy at an ultrasound intensity of about 100 mW/cm² to about 6,000 mW/cm². In some embodiments, the applying ultrasonic acoustic energy to the tissue comprises applying the ultrasound acoustic energy at an ultrasound intensity of about 100 mW/cm² to about 500 mW/cm². In some embodiments, the applying ultrasonic acoustic energy to the tissue comprises applying the ultrasound acoustic energy at an ultrasound intensity of about 110 mW/cm² to about 200 mW/cm². In some embodiments, the applying ultrasonic acoustic energy to the tissue comprises applying the ultrasound acoustic energy at an ultrasound intensity of about 188 mW/cm². In some embodiments, the ultrasound intensity is a spatial-peak temporal average intensity. In some embodiments, the acoustic radiation force is applied in two or more pulses, with an interval between each of the two or more pulses. In some embodiments, a plane wave ultrasound is applied to the tissue during the interval. In some embodiments, a B-mode ultrasound is applied to the tissue during the interval. In some embodiments a pulse duration of the two or more pulses is at least 20 microseconds. In some embodiments a pulse duration of the two or more pulses is at least 200 microseconds. In some embodiments, the ARF is applied at an ultrasound pulse length of at least 200 μs In some embodiments a pulse duration of the two or more pulses is about 10 microseconds to about 3300 microseconds. In some embodiments a pulse duration of the two or more pulses are about 50 microseconds to about 2000 microseconds. In some embodiments a pulse duration of the two or more pulses is up to 500 microseconds. In some embodiments a pulse duration of the two or more pulses is at least 100 microseconds. In some embodiments a pulse duration of the two or more pulses is about 100 microseconds to about 500 microseconds. In some embodiments a pulse duration of the two or more pulses is about 100 microseconds to about 600 microseconds. In some embodiments the interval is up to 500 milliseconds. In some embodiments the interval is up to 100, 500, 1000, 1500, 2000, 2500, 3000, 4000, or 5000 milliseconds. In some embodiments the interval is from about 100 milliseconds to about 5000 milliseconds. In some embodiments a sum of a pulse duration of the two or more pulses and the interval comprises a pulse repetition period. In some embodiments the pulse repetition period is at least 5 milliseconds (ms). In some embodiments the pulse repetition period is up to 5000 ms. In some embodiments the pulse repetition period is 5-5000 ms. In some embodiments the pulse repetition period is 20-2000 ms. In some embodiments the pulse repetition period is 1000-2000 ms. In some embodiments, the pulse repetition period is 100-5000 ms. In some embodiments the acoustic radiation force is applied at a pulse repetition frequency of 0.05 to 500 Hz. In some embodiments the acoustic radiation force is applied at a pulse repetition frequency of 0.05 to 250 Hz In some embodiments the acoustic radiation force is applied at a pulse repetition frequency of 0.1 to 100 Hz. In some embodiments the acoustic radiation force is applied at a pulse repetition frequency of 0.1 to 50 Hz. In some embodiments the acoustic radiation force is applied at a pulse repetition frequency of 0.5 to 50 Hz. In some embodiments, a time between application of the one or more sequences is at least 5, 10, 20, 30, 60, 120, 180, 240, 300, 360, 420, 480, 540, or 600 seconds. In some embodiments, a time between application of the one or more sequences ranges from about 5 to about 300 seconds. In some embodiments, a time between application of the one or more sequences ranges from about 10 to about 60 seconds. In some embodiments, the acoustic radiation force is applied for at least 10, 20, 30, 60, 120, 180, 240, 300, 360, 420, 480, 540, or 600 seconds. In some embodiments, the acoustic radiation force is applied for up to 30, 45, 60, 75, or 90 minutes. In some embodiments, applying ultrasonic acoustic energy to the tissue comprises applying the ultrasound acoustic energy at a focal depth of up to 10, 8, 6, or 4 cm from the ultrasound transducer. In some embodiments, applying ultrasonic acoustic energy to the tissue comprises applying the ultrasound acoustic energy at a focal depth of about 1 to about 10 cm from the ultrasound transducer. In some embodiments, applying ultrasonic acoustic energy to the tissue comprises applying the ultrasound acoustic energy at a focal depth of about 4 to about 10 cm from the ultrasound transducer. In some embodiments, applying ultrasonic acoustic energy to the tissue comprises applying the ultrasound acoustic energy at a focal depth of about 4 cm from the ultrasound transducer. In some embodiments, applying ultrasonic acoustic energy to the tissue comprises applying the ultrasound acoustic energy at a focal depth of about 6 cm from the ultrasound transducer. In some embodiments, the ARF is applied at duty cycle of less than 5%. In some embodiments, the ARF is applied at duty cycle of less than 4, 3, 2, or 1.5%. In some embodiments the applying ultrasonic acoustic energy to the tissue comprises applying the ultrasound acoustic energy at duty cycle of less than 1%. In some embodiments the applying ultrasonic acoustic energy to the tissue comprises applying the ultrasound acoustic energy at duty cycle of up to 0.1%. In some embodiments the applying ultrasonic acoustic energy to the tissue comprises applying the ultrasound acoustic energy at duty cycle of 0.01%-1.0%. In some embodiments the applying ultrasonic acoustic energy to the tissue comprises applying the ultrasound acoustic energy at duty cycle of 0.05%-1.0%.

In some embodiments, applying the plane wave or the B-mode ultrasound results in moving the sonoactive microstructures towards an endothelial border of the tissue comprising the cell. In some embodiments, the shear waves induce inertial cavitation the sonoactive microstructures at an endothelial border of the tissue comprising the cell, thereby enhancing delivery of the nucleic acid to the cell.

In some embodiments, the method includes applying a focused wave ultrasound to the tissue. In some embodiments, the method includes receiving focused wave ultrasound reflected from the tissue with the ultrasound probe. In some embodiments, the method includes displaying imaging data received from the reflected focused wave ultrasound. In some embodiments, the focused wave ultrasound is applied at a MI of up to 0.4. In some embodiments, the focused wave ultrasound is applied at a MI of greater than 0.4. In some embodiments, the focused wave ultrasound is applied at a MI of greater than 0.4 to about 3.0. In some embodiments, the focused wave ultrasound is applied at a MI of about 1.4. In some embodiments, the focused wave ultrasound is applied at a frequency of at least 0.1 MHz. In some embodiments, the focused wave ultrasound is applied at a frequency of about 0.1 MHz to about 20 MHz. In some embodiments, the focused wave ultrasound is applied at a frequency of about 0.1 MHz to about 10 MHz. In some embodiments, the focused wave ultrasound is applied at a frequency of about 2.5 MHz. In some embodiments, the focused wave ultrasound is applied at a duty cycle of up to 5%.

In some embodiments, the method further includes delivering a second or subsequent dose of the exogenous payload to the subject; and applying a second or subsequent acoustic radiation force (ARF) to the subject. In some embodiments, the second or subsequent dose of the exogenous payload are the same as a first dose of the exogenous payload. In some embodiments, the second or subsequent acoustic radiation force (ARF) is the same as the acoustic radiation force (ARF). In some embodiments, the second or subsequent acoustic radiation force (ARF) is applied at least 4 hours after the acoustic radiation force (ARF). In some embodiments, the second or subsequent dose of the exogenous payload is administered at least 4 hours after the first dose of the exogenous payload. In some embodiments, delivering the second or subsequent dose of the exogenous payload to the subject; and applying the second or subsequent acoustic radiation force (ARF) to the subject enhances delivery of the exogenous payload to the cell in the tissue of an organ of the subject, or enhances delivery of the exogenous payload to the cell in the tissue of an organ of the subject relative to a single administration.

In some embodiments, the method further includes sedating the subject. In some embodiments, the method further includes sedating the subject enhances delivery of the exogenous payload to the cell in the tissue of an organ of the subject.

In some embodiments, the exogenous payload comprises a nucleic acid payload encoding FVIII. In some embodiments, the method further includes delivering a second or subsequent dose of the exogenous payload to the subject; and applying a second or subsequent acoustic radiation force (ARF) to the subject. In some embodiments, a therapeutic level of FVIII is present in the subject's plasma following delivering the exogenous payload to the subject, and applying the acoustic radiation force (ARF) to the subject. In some embodiments, the therapeutic level of FVIII is achieved within 72 hours following delivering the exogenous payload to the subject, and applying the acoustic radiation force (ARF) to the subject.

In some embodiments, applying the focused wave ultrasound results in moving the sonoactive microstructures towards an endothelial border of the tissue comprising the cell. In some embodiments, the shear waves induce inertial cavitation the sonoactive microstructures at an endothelial border of the tissue comprising the cell, thereby enhancing delivery of the nucleic acid to the cell.

In some embodiments, the acoustic radiation force increases internalization of the exogenous payload in the cell. In some embodiments, the shear waves increase internalization of the exogenous payload in the cell. In some embodiments, inducing inertial cavitation the sonoactive microstructures increases internalization of the exogenous payload in the cell. In some embodiments, the ultrasound probe comprises a curved array probe, optionally, wherein the curved array probe is a C1-6 ultrasound probe. In some embodiments, the ultrasound probe is a phased array ultrasound probe, a linear ultrasound probe, a curvilinear ultrasound probe, a convex array ultrasound probe, an endocavitary ultrasound probe, a 3D ultrasound probe, a 4D ultrasound probe, a Doppler ultrasound probe, or a color doppler ultrasound probe.

In some embodiments, the payload comprises a nucleic acid construct. In some embodiments, the payload comprises an antibody, a small molecule drug, a protein, a cell therapy, a viral vector, or a peptide. In some embodiments, the nucleic acid construct comprises a therapeutic transgene, a DNA, ASO, a snRNA, an miRNA, an mRNA, a circular RNA, a self-amplifying RNA, an siRNA, a shRNA, a gRNA, a nucleic acid encoding a TALEN, a molecule encoding a zinc-finger nuclease, donor DNA, a transposase, an integrase, a retrotransposase, a saliogase, a base editor, a prime editor, a molecule encoding a CRISPR-associated protein 9, or a nucleic acid comprising a Clustered Regularly Interspaced Short Palindromic Repeats (CRISPR) sequence. In some embodiments, the nucleic acid construct comprises a therapeutic transgene, wherein the therapeutic transgene encodes Factor VIII, Factor IX, COL4A3, COL4A4, COL4A5, PKD1, or PKD2. In some embodiments, the organ is a liver, kidney, heart, brain, pancreas, skeletal muscle or smooth muscle, bone, or brain. In some embodiments, the cell is a hepatic cell, renal cell, pancreatic cell, myocyte, cardiac cell, blood cell, a hematopoietic cell, a tumor cell, a neuron, a glial cell, an immune cell, or a skeletal muscle or smooth muscle cell.

In some embodiments, the method comprises administering ultrasound energy transcutaneously to the subject in proximity to one or more target cells. In some embodiments, the one or more target cells are hepatic cells. In some embodiments, the one or more target cells are renal cells. In some embodiments, the one or more target cells are pancreatic cells. In some embodiments, the one or more target cells are cardiac cells. In some embodiments, the one or more target cells are myocytes. In some embodiments, the one or more target cells are neuronal cells. In some embodiments, the one or more target cells are brain cells. In some embodiments, the one or more target cells are blood cells (e.g., white blood cells). In some embodiments, the target cells are cancerous cells.

In some embodiments, the one or more target cells are in a tissue. In some embodiments, the tissue is skeletal muscle or smooth muscle tissue. In some embodiments, the tissue is smooth muscle tissue. In some embodiments, the tissue is connective tissue. In some embodiments, the tissue is lymphatic tissue. In some embodiments, the tissue is nervous tissue. In some embodiments, the tissue is diseased tissue, e.g., cancerous tissue, fibrotic tissue, or tissue otherwise in need of gene therapy.

In some embodiments, a nucleic acid payload comprises a regulatory element such as a promoter, (e.g., APOE-ATT, CAG). In some embodiments, a total amount (e.g., dose) of nucleic acid (e.g., DNA) administered to a subject for purposes of sonoporation can range from 100 microgram to 200 mg.

In some embodiments, the therapeutic payload is a nonendogenous gene. In some embodiments, the nucleic acid payload is configured to perform gene augmentation, gene replacement, gene editing, gene knockdown, or gene knockout.

In some embodiments, the nucleic acid construct comprises one or more regulatory elements, such as a promoter, enhancer, ribosome binding site, or transcription termination signal. Examples of promoters contemplated herein include, but are not limited to, e.g., CMV promoter, UbC promoter, CAG promoter, EF-1α promoter, ApoE promoter, ApoE-AAT1 promoter, 3XSERP promoter, or P3-hybrid promoter. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising SERP. In some embodiments, the nucleic acid construct comprises a promoter sequence comprising P3.

In some embodiments, inducing expression of the nucleic acid payload comprises inducing production of RNA encoded by the payload. In some embodiments, inducing expression of the nucleic acid payload comprises inducing production of protein encoded by the payload.

In some embodiments, the payload comprises a therapeutic RNA. In some embodiments, the therapeutic RNA is an mRNA. In some embodiments, the therapeutic RNA is an RNA interference (RNAi) agent, e.g., a double-stranded RNA, a single-stranded RNA, a micro RNA (miRNA), a short interfering RNA (siRNA), short hairpin RNA (shRNA), or a triplex-forming oligonucleotide. In some embodiments, the therapeutic RNA is a catalytically active RNA molecule (ribozyme). In some embodiments, the therapeutic RNA is a transfer RNA (tRNA). In some embodiments, the therapeutic RNA comprises one or more chemical modifications (e.g., one or more modified nucleobases, nucleosides, or nucleotides). In some embodiments, the nucleic acid construct is configured to perform gene augmentation, gene replacement, base editing, base knockdown, gene editing gene knockdown, or gene knockout. In some embodiments, delivering the nucleic acid payload to the target cell of the subject increases or decreases expression of a gene in the target cell.

In some embodiments, the payload comprises one or more components of a gene editing system. In some embodiments, the payload comprises a nuclease or engineered nuclease suitable for gene editing. In some embodiments, the nuclease is delivered as a polypeptide. In some embodiments, the nuclease is delivered as a nucleic acid encoding the nuclease. In some embodiments, the gene editing system is a CRISPR/Cas system. In some embodiments, the payload comprises a gRNA or a nucleic acid molecule encoding a gRNA (e.g., a plasmid encoding the gRNA). In some embodiments, the payload comprises a Cas protein or homologs or variants thereof, or a nucleic acid molecule encoding the Cas protein or homologs or variants thereof. In some embodiments, the payload comprises a TALEN or a nucleic acid molecule encoding the TALEN. In some embodiments, the payload comprises a zinc-finger nuclease (ZFN) or a nucleic acid encoding the ZFN. In some embodiments, the nuclease is an engineered nuclease. In some embodiments, the engineered nuclease is catalytically inactive. In some embodiments, the engineered nuclease is a fusion protein comprising the engineered nuclease, a regulatory protein, or an enzyme, or a functional domain thereof (e.g., a nuclease fused to a transcriptional regulatory domain or a nuclease fused to a deaminase) In some embodiments, the payload may further comprise a template DNA molecule suitable for knock-in to the subject's genome via non-homologous end joining (NHEJ) or homology directed repair (HDR).

Sonoactive microstructures (also referred to as acoustic microspheres or “microbubbles”) contemplated herein include, but are not limited to, those used as ultrasonic imaging contrast agents. In some embodiments, the sonoactive microstructures comprise a phospholipid-stabilized microstructure. In some embodiments, the phospholipid-stabilized microstructure comprises a high molecular weight gas core, or a perflutran core. Examples of sonoactive microstructures include, but are not limited to, OPTISON (GE Healthcare), Sonazoid (GE Healthcare), or DEFINITY and Definity RT (Lantheus Medical Imaging, Inc). In some embodiments, the sonoactive microstructures are LUMASON (Bracco) (sulfur hexafluoride lipid-type A microspheres). In some embodiments, the sonoactive microstructures are SonoVue (sulfur hexafluoride microbubbles). In some embodiments, the sonoactive microstructures comprise a protein-stabilized microstructure. In some embodiments, the sonoactive microstructures are Optison microbubbles.

The sonoactive microstructures can be administered prior to, after, or simultaneous (e.g., co-administered) with the administration of the nucleic acid construct (or nucleic acid payload). In some embodiments, the nucleic acid construct and the sonoactive microstructures are coadministered. In some embodiments, the administering of the nucleic acid construct and the sonoactive microstructures occurs serially, concurrently, sequentially, or continuously. In some embodiments, the administering of the nucleic acid construct and the sonoactive microstructures occurs serially. In some embodiments, the administering of the nucleic acid construct and the sonoactive microstructures occurs concurrently. In some embodiments, the administering of the nucleic acid construct and the sonoactive microstructures occurs sequentially. In some embodiments, the administering of the nucleic acid construct and the sonoactive microstructures occurs continuously.

In some embodiments, the nucleic acid construct is administered at a dosage of about 0.5 mg/kg to about 500 mg/kg. In some embodiments, about 2×10¹³ to about 3×10¹³ copies of the nucleic acid construct are administered to the subject. In some embodiments, each nucleic acid construct comprises a copy of a transgene.

As used herein, concentrations of microstructures/mL refers to the concentration of the sonoactive microstructures in a pharmaceutical composition immediately prior to administration to the subject. In some embodiments, the sonoactive microstructures are administered at a concentration of about 5×10⁸ to about 1.2×10¹⁰ microstructures/mL. In some embodiments, the sonoactive microstructures are administered at a dosage of about 1-50 mL, for example 1 mL of a protein-stabilized sonoactive microstructure (e.g., Optison). In some embodiments, the protein-stabilized sonoactive microstructure (e.g., Optison) has a diameter of 3-4.5 micrometers. The sonoactive microstructures may be administered at a concentration of about 5M (million) to about 8M microstructures per mL. In some embodiments, 1×10⁹ of phospholipid stabilized sonoactive microstructures (e.g., Sonazoid) are administered. In some embodiments, the phospholipid stabilized sonoactive microstructures (e.g., Sonazoid) comprise a diameter of 1-5 micrometers. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of at least 5×10\hat{ }8 microstructures per mL. In some embodiments, the sonoactive microstructures are administered at a concentration of up to 1.2×10\hat{ }10 microstructures/mL. In some embodiments, the sonoactive microstructures are administered at a concentration of 5×10\hat{ }8 to 8×10\hat{ }8 microstructures/mL.

In some embodiments, the nucleic acid construct and the sonoactive microstructures are mixed prior to being coadministered. In some instances, the sonoactive microstructures are mixed with the nucleic acid constructs before administering to the subject. In some instances, the sonoactive microstructures are mixed with the nucleic acid constructs along with additional buffers or agents such as saline or other biocompatible solutions with varying electrostatic charges and surface chemistries and ligands before administering to the subject. For example, Optison sonoactive microstructures can be mixed with a Nanoplasmid comprising APOE-Fluc and saline and administered together.

In some embodiments, the administering of the nucleic acid construct and the sonoactive microstructures is by intravenous administration or subcutaneous or intramuscular or intra-arterial or inter-osseous, or direct organ puncture. In some embodiments, after administering of the nucleic acid construct and sonoactive microstructures, the ultrasound acoustic energy is applied at the target cell, tissue, or organ.

A sonoporation treatment using the methods described herein can be used to treat a subject in need for gene therapy or enzyme replacement treatment. In another aspect, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher's (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II.

In some embodiments, the present disclosure provides methods of treating a subject having a liver condition with a therapeutic transgene. In some embodiments, the therapeutic transgene encodes one or more of: ATP7B; ABCB11; ABCB4; ATP8B1; TJP2; VWF; FVIII; FIX; F5; MAN2B1; GBA; SMPD1; CPS1; GDE/AGL; CTNS; SERPINA1; ASS1, and/or SLC25A13.

In some embodiments, the present disclosure provides methods of treating a subject having a liver condition with a therapeutic transgene. In some embodiments, the liver condition is Wilson's Disease, and the therapeutic transgene encodes ATP7B. In some embodiments, the liver condition is Cholestasis, progressive familial intrahepatic (PFIC1-4) and the therapeutic transgene encodes one or more of ABCB11, ABCB4, ATP8B1 and/or TJP2. In some embodiments, the liver condition is Von Willebrand Disease and the therapeutic transgene encodes VWF. In some embodiments, the liver condition is Hemophilia A, and the therapeutic transgene encodes FVIII. In some embodiments, the liver condition is Hemophilia B, and the therapeutic transgene encodes FIX. In some embodiments, the liver condition is Factor V Deficiency, and the therapeutic transgene encodes F5. In some embodiments, the liver condition is Alpha-Mannosidosis, and the therapeutic transgene encodes MAN2B1. In some embodiments, the liver condition is Gaucher's (glucocerebrosidase deficiency, glucocerebrosidosis), and the therapeutic transgene encodes GBA. In some embodiments, the liver condition is Niemann Pick Disease A/B, and the therapeutic transgene encodes SMPD1. In some embodiments, the liver condition is Carbamoylphosphate Synthetase I Deficiency, and the therapeutic transgene encodes CPS1. In some embodiments, the liver condition is Glycogen Storage Disease Type III, and the therapeutic transgene encodes GDE/AGL. In some embodiments, the liver condition is Cystinosis, and the therapeutic transgene encodes CTNS. In some embodiments, the liver condition is A1AT deficiency, and the therapeutic transgene encodes SERPINAL. In some embodiments, the liver condition is Citrullinemia Type I & II, and the therapeutic transgene encodes one or more of ASS1 and/or SLC25A13. In some embodiments, the methods comprise (a) administering to the subject a nucleic acid construct comprising the nucleic acid payload (e.g., a therapeutic transgene); (b) administering to the subject a plurality of sonoactive microstructures; and (c) administering a sonoporation treatment.

The present disclosure provides ultrasound systems comprising computer systems that are programmed to implement methods of the disclosure. The ultrasound systems 200 may be operably connected to one or more ultrasound transducers 211 controlled by a computer system 201 one or more computer processers 204 which may comprise one or more computer readable medium/media 205 which comprise instructions configured to cause the ultrasound systems to perform the methods of the present disclosure. The ultrasound systems 200 and/or the computer processers 204 may be in communication with the cloud 207 or other remote server which enable the remote operation and control of the ultrasound systems 200 and performance of the methods disclosed herein. The computer system 201 can be an electronic device of a user or a computer system that is remotely located with respect to the electronic device. The electronic device can be a mobile electronic device. The computer system includes a central processing unit (CPU, also “processor” and “computer processor” herein), which can be a single core or multi core processor, or a plurality of processors for parallel processing. The computer system also includes memory or memory location 206 (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., hand-held device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, concentration of the analyte of interest. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit.

In many instances, systems, platforms, software, networks, and methods described herein include a digital processing device, or use of the same. In further embodiments, the digital processing device includes one or more hardware central processing units (CPUs), i.e., processors that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In other embodiments, the digital processing device could be deployed on premise or remotely deployed in the cloud.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In many aspects, the systems, platforms, software, networks, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Aspects disclosed herein provide a system comprising: an ultrasound transducer configured to apply a focused acoustic radiation force (ARF) to a tissue of a subject and generate shear waves in the tissue of the subject; a computer system comprising a computer processor and a computer-readable medium configured to implement a method of applying the focused ARF and generate the shear waves in the tissue of the subject by applying ultrasonic acoustic energy with the ultrasound transducer, the method comprising applying the ARF with ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof, wherein the subject has been administered the exogenous payload and a sonoactive agent, and the method enhance delivery of an exogenous payload to the tissue of the subject. Aspects disclosed herein provide a computer-readable medium comprising instructions which, when executed by a computer, cause the computer to carry out a method of applying a focused acoustic radiation force with ultrasonic acoustic energy to a tissue of a subject and generating shear waves in the tissue of the subject to enhance delivery of an exogenous payload to the tissue of a subject that has been administered with the exogenous payload and a sonoactive agent, the method comprising: applying the focused ARF with an ultrasound transducer applying ultrasound acoustic energy at one or more of: an ultrasound intensity of 100-5000 mW/cm2; a pulse length of greater than 200 μs (microseconds) and at an ultrasound intensity of up to 5000 mW/cm2; a duty cycle of less than 5% and a pulse length of greater than 200 us; an ultrasound intensity of at least 100 mW/cm2 and duty cycle of less than 1%; or combinations thereof.

In some embodiments, administering the payload and the sonoactive agent to the subject, and applying the acoustic radiation force (ARF) is sufficient to deliver at least 1 copy per nanogram of subject DNA throughout the organ, optionally, wherein the payload is DNA. In some embodiments, the ARF is applied at an ultrasound intensity of 100-5000 mW/cm2. In some embodiments, the ARF is applied at an ultrasound pulse length of at least 200 us. In some embodiments, the ARF is applied at duty cycle of less than 5%. In some embodiments, the ARF is applied at duty cycle of less than 4, 3, 2, or 1.5%. In some embodiments, the ARF is applied at an ultrasound pulse length of at least 20 us. In some embodiments, the payload is an exogenous payload, and wherein the ARF generates shear waves in a tissue of the organ of the subject. In some embodiments, the payload is expressed in the cell at an expression level of at least 1.25, 1.5, 2, 3, 4, 5, 10, 15, 20, 25, 30, 35, 40, 45, 50, 75, 100, 150, 200, 250, or 500 copies per nanogram of subject DNA throughout the organ, optionally, wherein the payload is DNA. In some embodiments, the payload is expressed throughout the organ in every lobe of the organ. In some embodiments, the payload is expressed throughout the organ in two samples of the organ taken from opposite ends of the organ. In some embodiments, the samples are samples sized up to 1 cm³ or up to 1 g. In some embodiments, the payload is expressed in the cell at an expression level of at least 0.005 copies per diploid genome. In some embodiments, the payload is expressed in the cell at an expression level of at least 0.01 copies per diploid genome. In some embodiments, the ARF is applied at an ultrasound intensity of at least 150, 175, 200, 225, 250, 275, 300, 350, 400, 450, 500, 600, 700, 800, 900, 1000, 1250, 1500, 1750, 2000, 2250, 2500, 2750, 3000, 3250, 3500, 3750, 4000, 4250, 4500, or 4750 mW/cm2. In some embodiments, the ARF is applied at an ultrasound intensity of up to 500, 600, 700, 800, 900, 1000, 1250, 1500, 1750, 2000, 2250, 2500, 2750, 3000, 3250, 3500, 3750, 4000, 4250, 4500, 4750, or 5000 mW/cm2. In some embodiments, the applying ultrasonic acoustic energy to the tissue comprises applying the ultrasound acoustic energy at an ultrasound intensity of at least 100 mW/cm². In some embodiments, the applying ultrasonic acoustic energy to the tissue comprises applying the ultrasound acoustic energy at an ultrasound intensity of about 100 mW/cm² to about 10,000 mW/cm². In some embodiments, the applying ultrasonic acoustic energy to the tissue comprises applying the ultrasound acoustic energy at an ultrasound intensity of about 100 mW/cm² to about 5,000 mW/cm². In some embodiments, the applying ultrasonic acoustic energy to the tissue comprises applying the ultrasound acoustic energy at an ultrasound intensity of up to 5,000 mW/cm². In some embodiments, the applying ultrasonic acoustic energy to the tissue comprises applying the ultrasound acoustic energy at an ultrasound intensity of about 100 mW/cm² to about 500 mW/cm². In some embodiments, the applying ultrasonic acoustic energy to the tissue comprises applying the ultrasound acoustic energy at an ultrasound intensity of about 110 mW/cm² to about 200 mW/cm². In some embodiments, the applying ultrasonic acoustic energy to the tissue comprises applying the ultrasound acoustic energy at an ultrasound intensity of about 188 mW/cm². In some embodiments, the ultrasound intensity is a spatial-peak temporal average intensity (Ispta). In some embodiments, the spatial-peak temporal average intensity is calculated in a focal region of the tissue. In some embodiments, the ARF is applied at an ultrasound pulse length of at least 20 us. The method any one of embodiments 1, 4, or 5, wherein a pulse length is about 100 microseconds to about 500 microseconds. In some embodiments, the ARF is applied at an ultrasound pulse length 200-5000 us. In some embodiments, the ARF is applied at an ultrasound pulse length of at least 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us. In some embodiments, the ARF is applied at an ultrasound pulse length of up to 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 us. In some embodiments, the ARF is a focused acoustic radiation force. In some embodiments, the acoustic radiation force is applied in two or more pulses, with an interval between each of the two or more pulses. In some embodiments, a plane wave ultrasound is applied to the tissue during the interval. In some embodiments, a B-Mode ultrasound is applied to the tissue during the interval. In some embodiments, the interval is up to 500 milliseconds. In some embodiments, the interval is up to 100, 500, 1000, 1500, 2000, 2500, 3000, 4000, or 5000 milliseconds. In some embodiments, the interval is from about 100 milliseconds to about 5000 milliseconds. In some embodiments, a sum of a pulse duration of the two or more pulses and the interval comprises a pulse repetition period. In some embodiments, the pulse repetition period is at least 5 milliseconds (ms). In some embodiments, the pulse repetition period is up to 5000 ms. In some embodiments, the pulse repetition period is 5-5000 ms. In some embodiments, the pulse repetition period is 20-2000 ms. In some embodiments, the pulse repetition period is 1000-2000 ms. In some embodiments, the pulse repetition period is 100-5000 ms. In some embodiments, the focused acoustic radiation force is applied at a pulse repetition frequency of 0.05 to 500 Hz. In some embodiments, the focused acoustic radiation force is applied at a pulse repetition frequency of 0.05 to 250 Hz In some embodiments, the focused acoustic radiation force is applied at a pulse repetition frequency of 0.1 to 100 Hz. In some embodiments, the focused acoustic radiation force is applied at a pulse repetition frequency of 0.1 to 50 Hz. In some embodiments, the focused acoustic radiation force is applied at a pulse repetition frequency of 0.5 to 50 Hz. method of any one of the preceding embodiments, wherein the focused acoustic radiation force is applied at a pulse repetition frequency of 0.5 to 5 Hz. In some embodiments, the ARF deforms the tissue of the subject. In some embodiments, the ultrasonic acoustic energy is applied at a mechanical index of greater than 0.4. In some embodiments, the ultrasonic acoustic energy is applied at a mechanical index of about 1.4. In some embodiments, the ultrasonic acoustic energy is applied at a mechanical index of at least 1.3. In some embodiments, the ultrasonic acoustic energy is applied at a mechanical index of greater than 0.4 up to about 3.0. In some embodiments, the ultrasonic acoustic energy is applied at a frequency of about 0.1 MHz to about 20 MHz. In some embodiments, the ultrasonic acoustic energy is applied at a frequency of about 1 MHz to about 20 MHz. In some embodiments, the ultrasonic acoustic energy is applied at a frequency of about 0.1 MHz to about 10 MHz. In some embodiments, the ultrasonic acoustic energy is applied at a frequency of about 18 MHz. In some embodiments, the ultrasonic acoustic energy is applied at a frequency of about 2.5 MHz. In some embodiments, the applying ultrasonic acoustic energy to the tissue comprises applying the ultrasound acoustic energy at duty cycle of up to 0.1%. In some embodiments, the applying ultrasonic acoustic energy to the tissue comprises applying the ultrasound acoustic energy at duty cycle of 0.01%-1.0%. In some embodiments, the applying ultrasonic acoustic energy to the tissue comprises applying the ultrasound acoustic energy at duty cycle of 0.05%-1.0%. In some embodiments, the sonoactive microstructure do not encapsulate the exogenous payload, optionally, wherein the exogenous payload is a nucleic acid. In some embodiments, the ARF is applied with ultrasound acoustic energy at a mechanical index of at least 1.9. In some embodiments, the ARF is applied with ultrasound acoustic energy is applied at a mechanical index of at least 2.1. In some embodiments, the ARF is applied at a thermal index of less than 1.0. In some embodiments, the ARF is applied at a thermal index of 0.01-1.0. In some embodiments, the ARF is applied at a thermal index of 0.1-1.0. In some embodiments, the application of the ARF does not increase the temperature of the tissue by more than 1 C. In some embodiments, the application of the ARF does not increase the temperature of the tissue by more than 0.5 C. In some embodiments, the application of the ARF does not increase the temperature of the tissue by more than 0.1 C. In some embodiments, the ARF displaces the tissue of the subject. In some embodiments, the tissue is displaced by at least 0.001 mm. In some embodiments, the tissue is displaced by at least 0.01 mm. In some embodiments, the tissue is displaced by at least 0.1 mm. In some embodiments, the tissue is displaced by at least 1 mm. In some embodiments, the tissue is displaced by 0.01-1 mm. In some embodiments, the shear waves displace the tissue of the subject. In some embodiments, the shear waves displace the tissue of the subject in a direction perpendicular to the direction of the application of the ultrasound acoustic energy. In some embodiments, the shear waves displace the tissue by at least 0.001 mm. In some embodiments, the shear waves displace the tissue by at least 0.01 mm. In some embodiments, the shear waves displace the tissue by 0.01-1 mm. In some embodiments, a displacement of the shear waves in the tissue is at least 0.001 mm. In some embodiments, a displacement of the shear waves in the tissue is at least 0.01 mm. In some embodiments, a displacement of the shear waves in the tissue is 0.01-1 mm. In some embodiments, the ARF is applied using ultrasound acoustic energy having a pulse length of greater than 200 μs (microseconds) and induces a peak negative pressure of at least 3,000 kPa in the tissue. In some embodiments, the ARF is applied using ultrasound acoustic energy at an ultrasound intensity of at least 100 mW/cm2 and a pulse length of greater than 20 μs (microseconds). In some embodiments, the ARF is applied using ultrasound acoustic energy at an ultrasound intensity of at least 100 mW/cm2 and a duty cycle of less than 4, 3, 2, 1, or 0.5%. In some embodiments, the ARF is applied using ultrasound acoustic energy having a pulse length of greater than 200 μs (microseconds) and induces a peak negative pressure of at least 3300, 3500, 4000, 4500, 5000, 5500, 6000, 6500, 7000, or 7500 kPa in the tissue. In some embodiments, the exogenous payload comprises a nucleic acid construct. In some embodiments, at least 10 mg of the nucleic acid construct is administered to the subject. In some embodiments, the exogenous payload comprises an antibody, a small molecule drug, a protein, a cell therapy, a viral vector, or a peptide. In some embodiments, the nucleic acid construct comprises a therapeutic transgene, a DNA, ASO, a snRNA, an miRNA, an mRNA, a circular RNA, a self-amplifying RNA, an siRNA, a shRNA, a gRNA, a nucleic acid encoding a TALEN, a molecule encoding a zinc-finger nuclease, donor DNA, a transposase, an integrase, a retrotransposase, a saliogase, a base editor, a prime editor, a molecule encoding a CRISPR-associated protein 9, or a nucleic acid comprising a Clustered Regularly Interspaced Short Palindromic Repeats (CRISPR) sequence. In some embodiments, the nucleic acid construct comprises a therapeutic transgene, wherein the therapeutic transgene encodes Factor VIII, Factor IX, COL4A3, COL4A4, COL4A5, PKD1, or PKD2. In some embodiments, the organ is a liver, kidney, heart, brain, pancreas, skeletal muscle or smooth muscle, smooth muscle, bone, or brain. In some embodiments, the cell is a hepatic cell, renal cell, pancreatic cell, myocyte, cardiac cell, blood cell, a hematopoietic cell, a tumor cell, a neuron, a glial cell, an immune cell, or a skeletal muscle or smooth muscle cell. In some embodiments, the focused acoustic radiation force is applied using an ultrasound probe applying ultrasound acoustic energy to the tissue. In some embodiments, the sonoactive agent comprises a plurality of sonoactive microstructures. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1. In some embodiments, the method further includes a second or subsequent dose of the exogenous payload to the subject; and applying a second or subsequent focused acoustic radiation force (ARF) to the subject. In some embodiments, the second or subsequent dose of the exogenous payload are the same as a first dose of the exogenous payload. In some embodiments, the second or subsequent acoustic radiation force (ARF) is the same as the acoustic radiation force (ARF). In some embodiments, the second or subsequent acoustic radiation force (ARF) is applied at least 4 hours after the acoustic radiation force (ARF). In some embodiments, the second or subsequent dose of the exogenous payload is administered at least 4 hours after the first dose of the exogenous payload. In some embodiments, the second dose of the exogenous payload and the second acoustic radiation force (ARF) are administered at least 24 hours after the first dose of the exogenous payload and the first ARF.

### I. Exemplary Embodiments

Among the exemplary embodiments are:
1. An exogenous payload and a sonoactive agent for use in a method of treating a subject having a genetic disorder requiring a gene therapy or a protein replacement therapy, the method comprising:
   a. administering to the subject the sonoactive agent and the exogenous payload, the exogenous payload comprising a nucleic acid for expression of the gene therapy or the protein replacement therapy; and
   b. applying an acoustic radiation force (ARF) and generating shear waves in a tissue of the subject with an ultrasound system comprising an ultrasound transducer applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof,
   c. thereby delivering the exogenous payload to the tissue of the subject, and inducing expression of the gene therapy or the protein replacement therapy in the tissue of the subject.
2. An exogenous payload for use in a method of treating a subject having a genetic disorder requiring a gene therapy or a protein replacement therapy, the method comprising:
   a. administering to the subject the exogenous payload, the exogenous payload comprising a nucleic acid for expression of the gene therapy or the protein replacement therapy, and administering to the subject a sonoactive agent; and
   b. applying an acoustic radiation force (ARF) and generating shear waves in a tissue of the subject with an ultrasound system comprising an ultrasound transducer applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof,
   c. thereby delivering the exogenous payload to the tissue of the subject, and inducing expression of the gene therapy or the protein replacement therapy in the tissue of the subject.
3. A sonoactive agent for use in a method of treating a subject having a genetic disorder requiring a gene therapy or a protein replacement therapy, the method comprising:
   a. administering to the subject the sonoactive agent, and administering to the subject an exogenous payload comprising a nucleic acid for expression of the gene therapy or the protein replacement therapy; and
   b. applying an acoustic radiation force (ARF) and generating shear waves in a tissue of the subject with an ultrasound system comprising an ultrasound transducer applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof,
   c. thereby delivering the exogenous payload to the tissue of the subject, and inducing expression of the gene therapy or the protein replacement therapy in the tissue of the subject.
4. A sonoactive agent for use in a method of preparing a cell in a tissue of a subject for the delivery of an exogenous payload to the cell, the method comprising:
   a. administering a sonoactive agent to the subject; and
   b. applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
5. A sonoactive agent for use in a method of preparing a cell in a tissue of a subject having been administered with a sonoactive agent for the delivery of an exogenous payload to the cell, the method comprising:
   applying an acoustic radiation force (ARF) to the tissue of the subject, thereby generating shear waves in the tissue of the subject, wherein the ARF forms at least one transient pore in a cell membrane of the cell, wherein the ARF is applied using ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
6. A method of preparing a cell in a tissue of a subject having been administered with a sonoactive agent for the delivery of an exogenous payload to the cell, the method comprising:
   a. applying an acoustic radiation force (ARF) to the tissue of the subject, thereby generating shear waves in the tissue of the subject, wherein the ARF forms at least one transient pore in a cell membrane of the cell, wherein the ARF is applied using ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
7. A method of preparing a cell in a tissue of a subject for the delivery of an exogenous payload to the cell, the method comprising:
   a. administering a sonoactive agent to the subject; and
   b. applying an acoustic radiation force (ARF) to the tissue of the subject, thereby generating shear waves in the tissue of the subject, wherein the ARF forms at least one transient pore in the cell membrane of the cell, wherein the ARF is applied using ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
8. Use of an exogenous payload and a sonoactive agent in preparation of a system for use in a method of treating a subject having a genetic disorder requiring a gene therapy or a protein replacement therapy, the method comprising:
   a. administering to the subject the sonoactive agent and the exogenous payload, the exogenous payload comprising a nucleic acid for expression of the gene therapy or the protein replacement therapy; and
   b. applying an acoustic radiation force (ARF) and generating shear waves in a tissue of the subject with an ultrasound system comprising an ultrasound transducer applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof,
   c. thereby delivering the exogenous payload to the tissue of the subject, and inducing expression of the gene therapy or the protein replacement therapy in the tissue of the subject.
9. Use of an exogenous payload in manufacture of a system for use in a method of treating a subject having a genetic disorder requiring a gene therapy or a protein replacement therapy, the method comprising:
   a. administering to the subject the exogenous payload, the exogenous payload comprising a nucleic acid for expression of the gene therapy or the protein replacement therapy, and administering to the subject a sonoactive agent; and
   b. applying an acoustic radiation force (ARF) and generating shear waves in a tissue of the subject with an ultrasound system comprising an ultrasound transducer applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof,
   c. thereby delivering the exogenous payload to the tissue of the subject, and inducing expression of the gene therapy or the protein replacement therapy in the tissue of the subject.
10. Use of a sonoactive agent in preparation of a system for use in a method of treating a subject having a genetic disorder requiring a gene therapy or a protein replacement therapy, the method comprising:
   a. administering to the subject the sonoactive agent, and administering to the subject an exogenous payload comprising a nucleic acid for expression of the gene therapy or the protein replacement therapy; and
   b. applying an acoustic radiation force (ARF) and generating shear waves in a tissue of the subject with an ultrasound system comprising an ultrasound transducer applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof,
   c. thereby delivering the exogenous payload to the tissue of the subject, and inducing expression of the gene therapy or the protein replacement therapy in the tissue of the subject.
11. Use of a sonoactive agent in preparation of a system for use in a method of preparing a cell in a tissue of a subject for the delivery of an exogenous payload to the cell, the method comprising:
   a. administering a sonoactive agent to the subject; and
   b. applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
12. Use of a sonoactive agent in preparation of a system for use in a method of preparing a cell in a tissue of a subject having been administered with a sonoactive agent for the delivery of an exogenous payload to the cell, the method comprising:
   applying an acoustic radiation force (ARF) to the tissue of the subject, thereby generating shear waves in the tissue of the subject, wherein the acoustic radiation force forms at least one transient pore in the cell membrane of the cell, wherein the ARF is applied using ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
13. A computer-readable medium comprising instructions which, when executed by a computer, cause the computer to carry out a method of applying an acoustic radiation force (ARF) with ultrasonic acoustic energy to a tissue of a subject and generating shear waves in the tissue of the subject to enhance delivery of an exogenous payload to the tissue of a subject that has been administered with the exogenous payload and a sonoactive agent, the method comprising:
   a. applying the ARF with an ultrasound transducer by applying ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
14. A system comprising:
   a. an ultrasound transducer configured to apply a focused acoustic radiation force (ARF) to a tissue of a subject and generate shear waves in the tissue of the subject; and
   b. a computer system comprising a computer processor and a computer-readable medium configured to implement a method of applying the focused ARF and generate the shear waves in the tissue of the subject by applying ultrasonic acoustic energy with the ultrasound transducer, the method comprising applying the ARF with ultrasound acoustic energy at one or more of:
      i. an ultrasound intensity of 100-5000 mW/cm2;
      ii. a pulse length of greater than 200 μs (microseconds) and at an ultrasound intensity of up to 5000 mW/cm2;
      iii. a duty cycle of less than 5% and a pulse length of greater than 200 us;
      iv. an ultrasound intensity of at least 100 mW/cm2 and duty cycle of less than 1%; or
      v. combinations thereof.
15. Any one of the preceding embodiments, wherein the method is sufficient to deliver at least 1 copy per nanogram of subject DNA throughout the organ, optionally, wherein the payload is DNA.
16. Any one of the preceding embodiments, wherein in the method, the ultrasound intensity is 100-5000 mW/cm2.
17. Any one of the preceding embodiments, wherein in the method, the pulse length is at least 200 μs.
18. Any one of the preceding embodiments, wherein in the method, the duty cycle is less than 5%.
19. Any one of the preceding embodiments, wherein in the method, the duty cycle is less than 4, 3, 2, or 1.5%,
20. Any one of the preceding embodiments, wherein in the method, the pulse length is at least 20 μs.
21. Any one of the preceding embodiments, wherein the ARF generates the shear waves in an organ of the subject, wherein the organ comprises the tissue.
22. Any one of the preceding embodiments, wherein in the method, the payload is expressed in the cell at an expression level of at least 1.25, 1.5, 2, 3, 4, 5, 10, 15, 20, 25, 30, 35, 40, 45, 50, 75, 100, 150, 200, 250, or 500 copies per nanogram of subject DNA throughout an organ comprising the tissue.
23. Any one of the preceding embodiments, wherein in the method, the expression throughout an organ comprising the tissue comprises inducing said expression in every lobe of the organ.
24. Any one of the preceding embodiments, wherein in the method, the expression throughout an organ comprising the tissue comprises inducing said expression in two samples of the organ taken from opposite ends of the organ.
25. Any one of the preceding embodiments, wherein in the method, the samples are samples sized up to 1 cm³ or up to 1 g.
26. Any one of the preceding embodiments, wherein in the method, the payload is expressed in the cell at an expression level of at least 0.005 copies per diploid genome.
27. Any one of the preceding embodiments, wherein in the method, the payload is expressed in the cell at an expression level of at least 0.01 copies per diploid genome.
28. Any one of the preceding embodiments, wherein in the method, the ultrasound intensity is at least 150, 175, 200, 225, 250, 275, 300, 350, 400, 450, 500, 600, 700,800, 900, 1000, 1250, 1500, 1750, 2000, 2250, 2500, 2750, 3000, 3250, 3500, 3750, 4000, 4250, 4500, or 4750 mW/cm2.
29. Any one of the preceding embodiments, wherein in the method, the ultrasound intensity is up to 500, 600, 700,800, 900, 1000, 1250, 1500, 1750, 2000, 2250, 2500, 2750, 3000, 3250, 3500, 3750, 4000, 4250, 4500, 4750, or 5000 mW/cm2.
30. Any one of the preceding embodiments, wherein in the method, the ultrasound intensity is at least 100 mW/cm².
31. Any one of the preceding embodiments, wherein in the method, the ultrasound intensity is about 100 mW/cm² to about 10,000 mW/cm².
32. Any one of the preceding embodiments, wherein in the method, the ultrasound intensity is about 100 mW/cm² to about 5,000 mW/cm².
33. Any one of the preceding embodiments, wherein in the method, the ultrasound intensity is up to 5,000 mW/cm².
34. Any one of the preceding embodiments, wherein in the method, the ultrasound intensity is about 100 mW/cm² to about 500 mW/cm².
35. Any one of the preceding embodiments, wherein in the method, the ultrasound intensity is about 110 mW/cm² to about 200 mW/cm²
36. Any one of the preceding embodiments, wherein in the method, the ultrasound intensity is about 188 mW/cm².
37. Any one of the preceding embodiments, wherein in the method, the ultrasound intensity is a spatial-peak temporal average intensity (Ispta).
38. The method of embodiment 31, wherein the spatial-peak temporal average intensity is provided at ultrasound source.
39. Any one of the preceding embodiments, wherein in the method, the ARF is applied at an ultrasound pulse length of at least 20 μs.
40. The method of any one of embodiments 1, 4, or 5, wherein the pulse length is about 100 microseconds to about 500 microseconds.
41. Any one of the preceding embodiments, wherein in the method, the pulse length is from about 200 μs to about 5000 μs.
42. Any one of the preceding embodiments, wherein in the method, the pulse length is at least 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 μs.
43. Any one of the preceding embodiments, wherein in the method, the pulse length is up to 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 6000, 7000, 8000, 9000, or 10000 μs.
44. Any one of the preceding embodiments, wherein in the method, the ARF is an acoustic radiation force.
45. Any one of the preceding embodiments, wherein in the method, the ARF is applied in two or more pulses, with an interval between each of the two or more pulses.
46. The method of embodiment 39, wherein a plane wave ultrasound is applied to the tissue during the interval.
47. The method of embodiment 39, wherein a B-Mode ultrasound is applied to the tissue during the interval.
48. The method of embodiment 39, wherein the interval is up to 500 milliseconds.
49. The method of embodiment 39, wherein the interval is up to 100, 500, 1000, 1500, 2000, 2500, 3000, 4000, or 5000 milliseconds.
50. The method of embodiment 39, wherein the interval is from about 100 milliseconds to about 5000 milliseconds.
51. Any one of the preceding embodiments, wherein in the method a sum of a pulse duration of the two or more pulses and the interval comprises a pulse repetition period.
52. Any one of the preceding embodiments, wherein in the method, a pulse repetition period of the ARF is at least 5 milliseconds (ms).
53. Any one of the preceding embodiments, wherein in the method, a pulse repetition period of the ultrasound acoustic energy is up to 5000 ms.
54. Any one of the preceding embodiments, wherein in the method, a pulse repetition period of the ultrasound acoustic energy is 5-5000 ms.
55. Any one of the preceding embodiments, wherein in the method, a pulse repetition period of the ultrasound acoustic energy is 20-2000 ms.
56. Any one of the preceding embodiments, wherein in the method, a pulse repetition period of the ultrasound acoustic energy is 1000-2000 ms.
57. Any one of the preceding embodiments, wherein in the method, a pulse repetition period of the ultrasound acoustic energy is 100-5000 ms.
58. Any one of the preceding embodiments, wherein in the method, the ARF is applied at a pulse repetition frequency of 0.05 to 500 Hz.
59. Any one of the preceding embodiments, wherein in the method, the ARF is applied at a pulse repetition frequency of 0.05 to 250 Hz
60. Any one of the preceding embodiments, wherein in the method, the ARF is applied at a pulse repetition frequency of 0.1 to 100 Hz.
61. Any one of the preceding embodiments, wherein in the method, the ARF is applied at a pulse repetition frequency of 0.1 to 50 Hz.
62. Any one of the preceding embodiments, wherein in the method, the ARF is applied at a pulse repetition frequency of 0.5 to 50 Hz.
63. Any one of the preceding embodiments, wherein in the method, the ARF is applied at a pulse repetition frequency of 0.5 to 5 Hz.
64. Any one of the preceding embodiments, wherein in the method, the ARF deforms the tissue of the subject.
65. Any one of the preceding embodiments, wherein in the method, the ultrasonic acoustic energy is applied at a mechanical index of greater than 0.4.
66. Any one of the preceding embodiments, wherein in the method, the ultrasonic acoustic energy is applied at a mechanical index of about 1.4.
67. Any one of the preceding embodiments, wherein in the method, the ultrasonic acoustic energy is applied at a mechanical index of at least 1.3.
68. Any one of the preceding embodiments, wherein in the method, the ultrasonic acoustic energy is applied at a mechanical index of greater than 0.4 up to about 3.0.
69. Any one of the preceding embodiments, wherein in the method, the ultrasonic acoustic energy is applied at a frequency of about 0.1 MHz to about 20 MHz.
70. Any one of the preceding embodiments, wherein in the method, the ultrasonic acoustic energy is applied at a frequency of about 1 MHz to about 20 MHz.
71. Any one of the preceding embodiments, wherein in the method, the ultrasonic acoustic energy is applied at a frequency of about 0.1 MHz to about 10 MHz.
72. Any one of the preceding embodiments, wherein in the method, the ultrasonic acoustic energy is applied at a frequency of about 18 MHz.
73. Any one of the preceding embodiments, wherein in the method, the ultrasonic acoustic energy is applied at a frequency of about 2.5 MHz.
74. Any one of the preceding embodiments, wherein in the method, the duty cycle is up to 0.1%.
75. Any one of the preceding embodiments, wherein in the method, the duty cycle is 0.01%-1.0%.
76. Any one of the preceding embodiments, wherein in the method, the duty cycle is 0.05%-1.0%.
77. Any one of the preceding embodiments, wherein in the method, a microstructure of the sonoactive agent does not encapsulate the exogenous payload, and optionally, wherein the exogenous payload is a nucleic acid.
78. Any one of the preceding embodiments, wherein in the method, the ultrasound acoustic energy is applied at a mechanical index of at least 1.9.
79. Any one of the preceding embodiments, wherein in the method, the ultrasound acoustic energy is applied at a mechanical index of at least 2.1.
80. Any one of the preceding embodiments, wherein in the method, the ARF is applied at a thermal index of less than 1.0.
81. Any one of the preceding embodiments, wherein in the method, the ARF is applied at a thermal index of 0.01-1.0.
82. Any one of the preceding embodiments, wherein in the method, the ARF is applied at a thermal index of 0.1-1.0.
83. Any one of the preceding embodiments, wherein in the method, the application of the ARF does not increase the temperature of the tissue by more than 1° C.
84. Any one of the preceding embodiments, wherein in the method, the application of the ARF does not increase the temperature of the tissue by more than 0.5° C.
85. Any one of the preceding embodiments, wherein in the method, the application of the ARF does not increase the temperature of the tissue by more than 0.1° C.
86. Any one of the preceding embodiments, wherein in the method, the ARF displaces the tissue of the subject.
87. Any one of the preceding embodiments, wherein in the method, the tissue is displaced by at least 0.001 mm.
88. Any one of the preceding embodiments, wherein in the method the tissue is displaced by at least 0.01 mm.
89. Any one of the preceding embodiments, wherein in the method, the tissue is displaced by at least 0.1 mm.
90. Any one of the preceding embodiments, wherein in the method, the tissue is displaced by at least 1 mm.
91. Any one of the preceding embodiments, wherein in the method, the tissue is displaced by 0.01-1 mm.
92. Any one of the preceding embodiments, wherein in the method, the shear waves displace the tissue of the subject.
93. Any one of the preceding embodiments, wherein in the method, the shear waves displace the tissue of the subject in a direction perpendicular to the direction of the application of the ultrasound acoustic energy.
94. Any one of the preceding embodiments, wherein in the method, the shear waves displace the tissue by or a displacement of the shear waves in the tissue is at least 0.001 mm.
95. Any one of the preceding embodiments, wherein in the method, the shear waves displace the tissue by or a displacement of the shear waves in the tissue is at least 0.01 mm.
96. Any one of the preceding embodiments, wherein in the method, the shear waves displace the tissue by or a displacement of the shear waves in the tissue is 0.01-1 mm.
97. Any one of the preceding embodiments, wherein in the method, the pulse length is greater than 200 μs (microseconds), and wherein the ARF induces a peak negative pressure of at least 3,000 kPa in the tissue.
98. Any one of the preceding embodiments, wherein in the method, the ultrasound intensity is at least 100 mW/cm2 and the pulse length is greater than 20 μs (microseconds).
99. Any one of the preceding embodiments, wherein in the method, the ultrasound intensity is at least 100 mW/cm2 and the duty cycle is less than 4, 3, 2, 1, or 0.5%.
100. Any one of the preceding embodiments, wherein in the method, the pulse length is greater than 200 μs (microseconds) and induces a peak negative pressure of at least 3300, 3500, 4000, 4500, 5000, 5500, 6000, 6500, 7000, or 7500 kPa in the tissue.
101. Any one of the preceding embodiments, wherein in the method, the exogenous payload comprises a nucleic acid construct.
102. Any one of the preceding embodiments, wherein in the method, at least 10 mg of a nucleic acid construct of the exogenous payload is administered to the subject.
103. Any one of the preceding embodiments, wherein in the method, the exogenous payload comprises an antibody, a small molecule drug, a protein, a cell therapy, a viral vector, or a peptide.
104. Any one of the preceding embodiments, wherein in the method, a nucleic acid construct of the exogenous payload comprises a therapeutic transgene, a DNA, ASO, a snRNA, an miRNA, an mRNA, a circular RNA, a self-amplifying RNA, an siRNA, a shRNA, a gRNA, a nucleic acid encoding a TALEN, a molecule encoding a zinc-finger nuclease, donor DNA, a transposase, an integrase, a retrotransposase, a saliogase, a base editor, a prime editor, a molecule encoding a CRISPR-associated protein 9, or a nucleic acid comprising a Clustered Regularly Interspaced Short Palindromic Repeats (CRISPR) sequence.
105. Any one of the preceding embodiments, wherein in the method, a nucleic acid construct of the exogenous payload comprises a therapeutic transgene, wherein the therapeutic transgene encodes Factor VIII, Factor IX, COL4A3, COL4A4, COL4A5, PKD1, or PKD2.
106. Any one of the preceding embodiments, wherein in the method, an organ comprising the tissue is a liver, kidney, heart, brain, pancreas, skeletal muscle, smooth muscle, bone, or brain.
107. Any one of the preceding embodiments, wherein in the method, the cell is a hepatic cell, renal cell, pancreatic cell, myocyte, cardiac cell, blood cell, a hematopoietic cell, a tumor cell, a neuron, a glial cell, an immune cell, a smooth muscle cell, or a skeletal muscle cell.
108. Any one of the preceding embodiments, wherein in the method, the ARF is applied using an ultrasound probe applying ultrasound acoustic energy to the tissue.
109. Any one of the preceding embodiments, wherein in the method, the sonoactive agent comprises a plurality of sonoactive microstructures.
110. Any one of embodiments 1-14, wherein the subject is a subject having Hemophilia A or FVIII deficiency, and the nucleic acid encodes FVIII.
111. Any one of embodiments 1-14, wherein the subject is a subject having Alport's Syndrome or COL4A5 deficiency, and the nucleic acid encodes alpha5(IV) chain of collagen IV.
112. Any one of embodiments 1-14, wherein the subject is a subject having PKD1 or polycystin-1 deficiency, and the nucleic acid encodes polycystin-1.
113. Any one of embodiments 1-14, the method further comprising: delivering a second or subsequent dose of the exogenous payload to the subject; and applying a second or subsequent acoustic radiation force (ARF) to the subject.
114. The method of embodiment 113, wherein the second or subsequent dose of the exogenous payload are the same as a first dose of the exogenous payload.
115. The method of embodiments 113-114 wherein the second or subsequent acoustic radiation force (ARF) is the same as the acoustic radiation force (ARF).
116. The method of embodiments 113-115, wherein the second or subsequent acoustic radiation force (ARF) is applied at least 4 hours after the acoustic radiation force (ARF).
117. The method of embodiments 113-116, wherein the second or subsequent dose of the exogenous payload is administered at least 4 hours after the first dose of the exogenous payload.
118. The method of embodiments 107-117, wherein the second dose of the exogenous payload and the second acoustic radiation force (ARF) are administered at least 24 hours after the first dose of the exogenous payload and the first ARF.

### II. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “SPTA,” or “I_{SPTA}” refers to spatial-peak temporal average intensity.

The term “intensity” refers to spatial-peak temporal average intensity unless otherwise stated.

A measure of “intensity” refers to the intensity value at the source of the ultrasound as applied by the ultrasound transducer unless otherwise stated.

The term “luc” refers to firefly luciferase.

The term “IVIS” refers to In Vivo Imaging System.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

### III. Examples

The following examples are included for illustrative purposes only and are not intended to limit the scope of the invention.

### Example I.: Sonoporation Using B-Mode Ultrasound in the Murine Liver

In this example, 4 C57BL/6 mice were studied in an experiment evaluating gene expression and durability of gene expression in the mouse liver. Mice were infused with protein stabilized sonoactive microstructures (Optison) and nucleic acids encoding a luciferase reporter gene coupled to a CAG promoter (pDNA-luc) (see, e.g., SEQ ID NOS. 3-4) were administered via a surgically implanted jugular vein catheter (JVC). Each mice was administered 120 μL of protein stabilized Optison sonoactive microstructures and 157.5 μg pDNA-luc. An acoustic contact agent (Aqua gel) was directly applied to the abdominal surface and the ultrasound acoustic energy was applied to the upper abdominal skin surface of the mice.

Ultrasound was applied using a GE LOGIQ linear array probe L6-24 to generate a B-mode ultrasound image using an MI of 0.3, then alternating mechanical indexes of 0.3 and 1.5, at a frequency of 7.87 MHz; the external ultrasound probe was applied to the upper right quadrant abdomen of the subjects for about 100 seconds. Low MI imaging (0.3) the liver was initiated for the initial 20 seconds following the infusion. The frequency of the low MI ultrasound was 7.87 MHz, and the pulse duration of the low MI ultrasound was 0.32 us. At 21 seconds, an ultrasound pulse at a high MI of 1.5 was applied for a pulse duration of 0.82 μs. The frequency of the high MI ultrasound was 6.2 MHz, and the pulse duration of the low MI ultrasound was 0.82 us. After the High MI mode, a Low MI imaging ultrasound was reapplied, and the High MI was implemented every 10 seconds for 9 times (total of about 90 seconds).

Following the procedure, each mouse underwent In Vivo Imaging System (IVIS) using bioluminescence imaging (BLI) receiving isoflurane sedation and a D-Luciferin injections to detect luciferase within the targeted organ (liver).

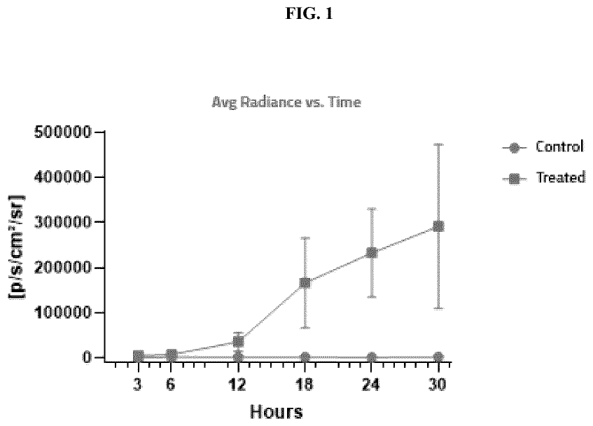

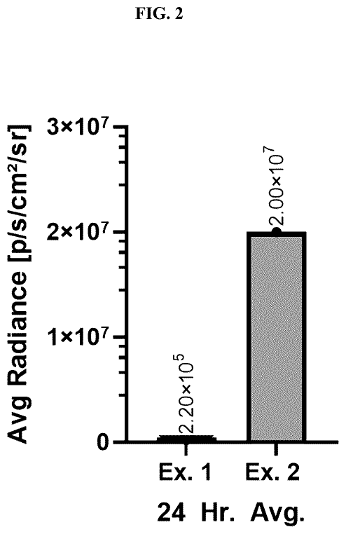

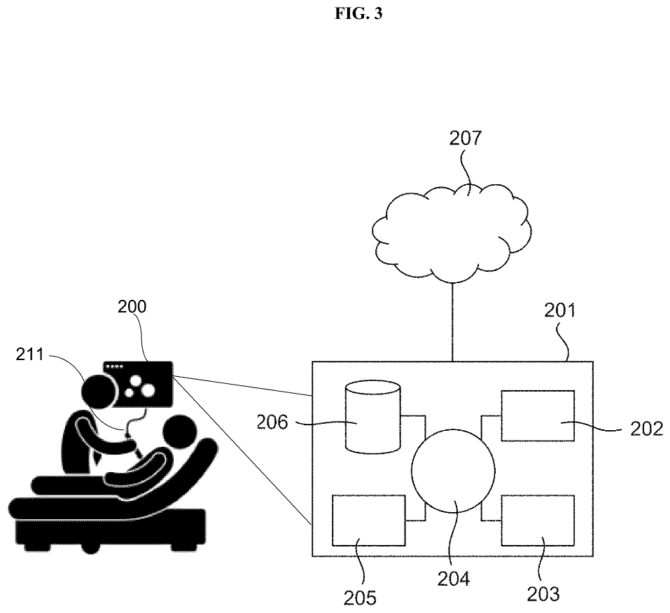

Results are shown in FIG. 1. IVIS imaging was performed at 3, 6, 12, 18, 24, and 30 hours. The gene expression across the 4 subjects were averaged, as is shown in FIG. 1. IVIS results revealed minimal observable fluorescence (p/s/cm²/sr), with a signal of less than 1E4 at 3 and 6 hours, a signal of about 4E4 at 12 hours, a signal of about 1.7E5 at 18 hours, 2.2E5 at 24 hours, and 3E5 at 30 hours. A control subject was administered 150 μL of protein stabilized Optison sonoactive microstructures and 250 μg pDNA-luc, and received no ultrasound application. The control subject exhibited no observable fluorescence at any IVIS imaging session.

### Example II.: Sonoporation Using Ultrasound Inducing Shear Waves in the Murine Liver

In this example, sonoporation utilizing ultrasound to apply an acoustic radiation force and induce shear waves in a mouse liver was investigated. Two wild type mice were infused about 0.2 mL of protein stabilized sonoactive microstructures (Optison) and nucleic acids encoding a luciferase reporter gene coupled to a CAG promoter (pDNA-luc) via a surgically implanted jugular vein catheter (JVC). In each treatment session, ultrasound was applied to the liver of each mouse with a GE LOGIQ e10 ultrasound system equipped with a C1-6 probe, and use of the “ELASTO” software, (Elastography) Shear Wave. Shear wave ultrasound acoustic radiation force protocol ultrasound was applied using 100% power (acoustic parameters included: LOGIQ e10 system with a C1-6 probe, Abdomen mode, MI of 1.4, Frequency 2.5 MHz, Gain 44, Depth 4.0 cm, AO % 100, gain 55, T 8, SVD 6.0, Ao % 100, Track Output 100%, f50-400 Hz in general mode, and at an ultrasound intensity of 117.5 to 187.9 mW/cm² (I_{SPTA}) (spatial-peak temporal average intensity), at a pulse length of about 600 us, a pulse repetition frequency of about 1.7 Hz, a pulse repetition period of about 0.58 s and a duty cycle of about 0.1%.

The first mouse was administered about 50 μL of protein stabilized Optison sonoactive microstructures and 100 μg pDNA-luc, and was imaged at 4, 24, and 48 hours. The ultrasound acoustic radiation force protocol was applied for 10 seconds on, followed by 10 seconds with no ultrasound applied, in a cycle, for a total of 2 cycles applying a total of 20 seconds of the ultrasound acoustic radiation force application. A rapid loss of ultrasound contrast (due to inertial cavitation of the sonoactive agent) occurred following the application of the acoustic radiation force.

The second mouse was administered about 150 μL of protein stabilized Optison sonoactive microstructures and 250 μg pDNA-luc, and was imaged at 24 hours. The ultrasound acoustic radiation force protocol was applied for 10 seconds on, followed by 10 seconds with no ultrasound applied, in a cycle, for a total of 5 cycles applying a total of 50 seconds of the ultrasound acoustic radiation force application. A rapid loss of ultrasound contrast (due to inertial cavitation of the sonoactive agent) occurred following the application of the acoustic radiation force.

Following the procedure, each mouse underwent IVIS imaging receiving isoflurane sedation and a D-Luciferin injections to detect luciferase within the targeted organ (liver). IVIS images were collected at 24 hours, and revealed a high signal over the abdomen in the mice treated with the shear wave ultrasound acoustic radiation force protocol.

The results are summarized in the below table.

IVIS Results (p/sec/cm2/sr)

| | |
|---|---|
| Example 2 Average | 2E7 |

Mice 1 and 2, each exhibited fluorescence in the range of 1.7E6 to 3.82E7 under IVIS imaging at 24 hours, respectively, with an average fluorescence of about 2.0E7. When compared to the subject of Example 1 that only reached a maximum fluorescence in the 3E5 at 30 hours, it can be established that the subjects treated with the ultrasound acoustic radiation force protocol as compared to standard ultrasound sonoporation techniques using standard imaging ultrasound exhibit significantly improved delivery of the nucleic acid payload to cells of the subject liver in the sonoporation treatment, and resulting gene expression, as shown by an increase in fluorescence signal generated by expression of firefly luciferase in the murine liver.

### Example III.: Sonoporation Using Shear Wave Ultrasound in the Murine Liver

In this example, sonoporation utilizing ultrasound to apply an acoustic radiation force and induce shear waves in a mouse liver was investigated. Six wild type mice were infused with protein stabilized sonoactive microstructures (Optison) or phospholipid stabilized sonoactive microstructures (Sonazoid), and nucleic acids encoding a luciferase reporter gene coupled to a CAG promoter (pDNA-luc) via a surgically implanted jugular vein catheter (JVC). In each treatment session, ultrasound was applied to the liver of each mouse with a GE LOGIQ e10 ultrasound system equipped with a C1-6 probe, and use of the “ELASTO” software, Shear Wave, using shear wave elastography parameters: GE LOGIQe10 probe C1-6, Abdomen, CHI, Frame rate 55, MI 1.4, Frequency 2.5 MHz, Gain 44, Depth 4 cm, AO %100, Gain 55, T 8, SVD 6.0, AO %100%, +50-400 Hz, GEN. 100% push output and 100% Track output.

Mice 1-3 were administered 150 μL of protein stabilized Optison sonoactive microstructures and 250 μg pDNA-luc, and an acoustic radiation force protocol was applied for 10 seconds, followed by 20 seconds with no application of ultrasound, followed again by 10 seconds of an acoustic radiation force protocol, up to the amount of total time of radiation force protocol application indicated in the below table. The shear wave ultrasound acoustic radiation force protocol was applied at an ultrasound intensity of 117.5 to 187.9 mW/cm² (I_{SPTA}) (spatial-peak temporal average intensity), at a mechanical index of 1.4, and a frequency of 2.5 MHz. Ultrasound energy was further applied to generate a B-mode ultrasound image at a mechanical index of 0.09, and a frequency of 2.5 MHz, at a pulse length of about 600 us, a pulse repetition frequency of about 1.7 Hz, a pulse repetition period of about 0.58 s and a duty cycle of about 0.1%. A rapid loss of ultrasound contrast (due to inertial cavitation of the sonoactive agent) occurred following the application of the acoustic radiation force.

Mice 4-6 were administered 150 μL of phospholipid stabilized Sonazoid sonoactive microstructures and 250 μg pDNA-luc, and an acoustic radiation force protocol was applied for 10 seconds, followed by 20 seconds with no application of ultrasound, followed again by 10 seconds of an acoustic radiation force protocol, up to the amount of total time of radiation force protocol application indicated in the below table. The shear wave ultrasound acoustic radiation force protocol was applied at an ultrasound intensity of 117.5 to 187.9 mW/cm² (I_{SPTA}) (spatial-peak temporal average intensity), at a mechanical index of 1.4, and a frequency of 2.5 MHz. Ultrasound energy was applied using a constant imaging technique applying ultrasound and generating a B-mode ultrasound image at a mechanical index of 0.09, and a frequency of 2.5 MHz, at a pulse length of about 600 us, a pulse repetition frequency of about 1.7 Hz, a pulse repetition period of about 0.58 s and a duty cycle of about 0.1%. A rapid loss of ultrasound contrast (due to inertial cavitation of the sonoactive agent) occurred following the application of the acoustic radiation force.

Following the procedure, each mouse underwent IVIS imaging receiving isoflurane sedation and a D-Luciferin injections to detect luciferase within the targeted organ (liver). IVIS images were collected at 4, and 24 hours, and revealed a high signal over the abdomen in the mice treated with the shear wave ultrasound acoustic radiation force protocol.

The experimental conditions and results are summarized in the below table.

| | IVIS | | | |
|---|---|---|---|---|
| | (p/sec/cm²/sr) | | | |
| Animal | Imaged at | Imaged at | Total time of | |
| number | 3-4 hours | 23-24 hours | ARF (sec) | Agent |
| 1 | 5E5 | 3E6 | 40 | Optison |
| 2 | 1E5 | 1E6 | 60 | Optison |
| 3 | 3E5 | 4E6 | 60 | Optison |
| 4 | 2E5 | 9E5 | 90 | Sonazoid |
| 5 | 1E4 | 7E5 | 60 | Sonazoid |
| 6 | 6E5 | 3E7 | 40 | Sonazoid |

Mice 1-3 exhibited fluorescence of in the range of 1E5 to 5E5 under IVIS imaging at 4 hours, and in the range of 1E6 to 4E6 under IVIS imaging at 24 hours, indicating a significant degree of indicating a significant increase in degree of gene transfection and expression when treated with the shear wave ultrasound acoustic radiation force protocol.

Mice 4-6 exhibited fluorescence of in the range of 1E4 to 6E5 under IVIS imaging at 4 hours, and in the range of 7E5 to 3E7 under IVIS imaging at 24 hours, indicating a significant degree of indicating a significant increase in degree of gene transfection and expression when treated with the shear wave ultrasound acoustic radiation force protocol.

When compared to the subject of Example 1, that only reached a maximum fluorescence in the 3E5 at 30 hours, it can be established that the subjects treated with the ultrasound acoustic radiation force protocol as compared to standard ultrasound sonoporation techniques exhibit significantly improved delivery of the nucleic acid payload to cells of the subject liver in the sonoporation treatment, and resulting gene expression, as shown by an increase in fluorescence signal generated by expression of firefly luciferase in the murine liver.

### Example IV.: Sonoporation Using Ultrasound Inducing Shear Waves on Sedated Subjects in the Murine Liver

In this example, sonoporation in sedated subjects utilizing ultrasound to apply an acoustic radiation force and induce shear waves in a mouse liver was investigated. Six RAG2 mice were infused with protein stabilized sonoactive microstructures (Optison), and 250 micrograms of nucleic acids encoding a tdTomato-luciferase reporter gene coupled to a CAG promoter (pDNA-CAG-tdTomato-Fluc) via a surgically implanted jugular vein catheter (JVC) in 50 microliters of PBS. Three of the six were sedated with intraperitoneal injections of ketamine at dosages of 200 mg/kg. In each treatment session, ultrasound was applied to the liver of each mouse with a GE LOGIQ e10 ultrasound system equipped with a C1-6 probe, and use of the “ELASTO” software, Shear Wave, using shear wave elastography parameters: GE LOGIQe10 probe C1-6, Abdomen, CHI, Frame rate 55, MI 1.4, Frequency 2.5 MHz, Depth 4 cm, AO %100, Gain 55, T 8, SVD 3.5, AO %100%, +50-400 Hz, GEN. 100% push output and 100% Track output, at a pulse length of about 600 us, a pulse repetition frequency of about 1.7 Hz, a pulse repetition period of about 0.58 s and a duty cycle of about 0.1%.

SWE ULTRASOUND PARAMETERS GE LOGIQ
e10 C1-6 probe with ELASTO software

| MI | 1.4 | |
|---|---|---|
| Tis | 0.3 | |
| Frame Rate | 55 | |
| CHI | | |
| Frequency | 2.5 | MHz |
| Gain | 44 | |
| AO % | 100 | |
| E | | |
| Gain | 55 | |
| T | 8 | |
| SVD | 3.5 | |
| PO % | 100% | |
| TO | 100% | |
| F | 50-400 | Hz |

| | |
|---|---|
| Penetration | — |
| ISPTA | 187.9 |

Mice 1-3 were administered 150 μL of protein stabilized Optison sonoactive microstructures and 250 μg pDNA-luc in 50 microliters of PB via the JVC, and an acoustic radiation force protocol was applied for 10 seconds, followed by 20 seconds with no application of ultrasound, followed again by 10 seconds of an acoustic radiation force protocol, for a total time of 40 seconds of acoustic radiation force protocol applied. The shear wave ultrasound acoustic radiation force protocol was applied at an ultrasound intensity of 187.9 mW/cm² (I_{SPTA}) (spatial-peak temporal average intensity), at a mechanical index of 1.4, and a frequency of 2.5 MHz. Ultrasound energy was further applied to generate a B-mode ultrasound image at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%.

Mice 4-6 were administered 150 μL of phospholipid stabilized Optison sonoactive microstructures and 250 μg pDNA-luc in 50 μL in PBS, and intraperitoneal injections of ketamine at dosages of 200 mg/kg and an acoustic radiation force protocol was applied for 10 seconds, followed by 20 seconds with no application of ultrasound, followed again by 10 seconds of an acoustic radiation force protocol, for a total time of 40 seconds of acoustic radiation force protocol applied. The shear wave ultrasound acoustic radiation force protocol was applied at an ultrasound intensity of 187.9 mW/cm² (I_{SPTA}) (spatial-peak temporal average intensity), at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%. Ultrasound energy was applied to generate a B-mode ultrasound image at a mechanical index of 1.4, and a frequency of 2.5 MHz. A rapid loss of ultrasound contrast (due to inertial cavitation of the sonoactive agent) occurred following the application of the acoustic radiation force.

Following the procedure, each mouse underwent IVIS imaging receiving isoflurane sedation and a D-Luciferin injections to detect luciferase within the targeted organ (liver). IVIS images were collected at 24 hours, and revealed a high signal over the abdomen in the mice treated with the shear wave ultrasound acoustic radiation force protocol, with mice having undergone ketamine sedation exhibiting increased luciferase signal.

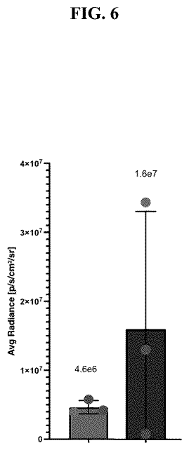

The experimental conditions and results are summarized in the below table, and in FIG. 6. It is observed that subjects sedated with ketamine exhibited significantly greater gene transfection and expression as compared to subjects un-sedated at the time of the sonoporation treatment.

| | | | IVIS |
|---|---|---|---|
| Mouse | | | (p/sec/cm2/sr) |
| number | Ketamine | Total time of | at 24 hours |
| RAG2 | dose | ARF (sec) | (average) |
| 1-3 | None | 40 | 4.6E6 |
| 4-6 | 200 mg/kg | 40 | 1.6E7 |

Following the IVIS imaging, necropsy is performed and subjects were evaluated using digital droplet polymer chain reaction (ddPCR) to assess copy number per diploid genome (CN/DG) levels of DNA payload delivered to the cell in the organ. The liver of two subject were split into the medial, right, caudate, left dorsal, left middle, and left ventricle lobes. The tissue as cleared of blood by sterile saline. Tissue was placed in Nase/DNase-free tubes (purchased certified or autoclaved) and snap-frozen in LN2 and transferred to −80 C storage. The procedure began by preparing the Master Mix (MM) for each reaction. First, the ddPCR Supermix for probes (no dUTP) was thawed and vortexed for at least 30 seconds. For each primer/probe set, 11 μL ddPCR Supermix, 1.1 μL primer/probe mix, 0.275 μL HindIlI enzyme, and water were combined to bring the final reaction volume to 18 μL. For single-plex reactions, 5.626 μL of water was added, while for duplex reactions, 4.525 μL was added. The Master Mix was calculated with a correction factor of 1.1 to account for pipetting error. After preparation, 18 μL of the Master Mix was dispensed into individual wells of a 96-well PCR plate, which was then set aside at room temperature. DNA samples were diluted using nuclease-free water to the desired concentration, and 4 μL of the diluted genomic DNA (gDNA) or ddFHO (for negative controls) was added to each well according to the plate map. The plate was then sealed with a pierceable foil seal, ensuring the red stripe faced up at the top, and vortexed for 2 minutes at room temperature. Following this, the plate was spun down at 100-300 rcf for 1 minute to remove bubbles. The sealed plate was placed in the Automated Droplet Generator (ADG), ensuring that the destination plate on a cooling block, DG32 cartridges, ddPCR tips (with lids removed), and an empty waste container were properly positioned. The droplet generation process was initiated, and the corresponding ddPCR program was selected on the thermal cycler, which was paused while waiting for the droplet generation to finish. Once the droplet generation was completed, the 96-well plate was removed from the cooling block, resealed with a pierceable foil seal, and transferred to the thermal cycler, where the cycling process was resumed. The cooling block was returned to −20° C. in an upside-down position for future use. Thermal cycling was performed based on the ddPCR program, which typically included steps such as denaturation, annealing, and extension, although specific conditions depended on the primer/probe set used. After thermal cycling, the plate was transferred to the Droplet Reader. In the ddPCR software, the appropriate Supermix and fluorophores (e.g., FAM, VIC, HEX) were selected before initiating the droplet reading process. Once the droplet reading was completed, the results provided the quantification of DNA copy number per diploid genome, as well as its relative abundance to reference genes. The software generated data based on the fluorescence signals detected in the droplets, enabling accurate measurement of the target gene copy numbers.

Results are shown in FIG. 14, where it is observed that each of the medial, right, caudate, left dorsal, left middle, and left ventricle lobes of the subject livers treated with the acoustic radiation force ultrasound exhibited an average copy number per diploid genome (CN/DG) ranging from 0.074 CN/DG to 0.108 CN/DG.

Results in CN/DG can be converted to copy number per nanogram of DNA (CN/ng) by use of the conversion factor of 0.006 ng of DNA per diploid genome. Applying this conversion factor, an average copy number per ng of genomic DNA (CN/ng) ranging from 12.33 CN/ng to 16.66 CN/ng can be obtained with respect to medial, right, caudate, left dorsal, left middle, and left ventricle lobes of the subject livers treated with the acoustic radiation force ultrasound.

When compared to a liver tissue biodistribution analysis conducted in Example XIV in which the murine lever was treated using a B-mode ultrasound protocol, it is observed that the ultrasound profile inducing shear waves in the tissue utilized in this Example IV significantly increased biodistribution of the nucleic acid payload in the treated organ. It is shown in FIG. 14 that each of the medial, right, caudate, left dorsal, left middle, and left ventricle lobes of the subject livers treated with the acoustic radiation force ultrasound exhibited an average copy number per diploid genome (CN/DG) ranging from 0.074 CN/DG to 0.108 CN/DG which was larger and highly uniform throughout the treated liver, whereas the B-mode ultrasound protocol treated liver from Example XIV in which exhibited copy numbers of only 0.04 CN/DG in the ventral section closets to the ultrasound probe which rapidly decreased in regions of the tissue further away from the ultrasound probe in the middle and dorsal sections of the treated liver which exhibited half or less than half of the copy numbers at 0.02 CN/DG or less indicating a significant loss of transfection efficiency as distance increased from the ultrasound source.

### Example V.: Sonoporation Using Ultrasound Inducing Shear Waves in Repeated Treatments in the Murine Liver

In this example, application of sonoporation in multiple treatments utilizing ultrasound to apply an acoustic radiation force and induce shear waves in a mouse liver was investigated. 9 wild type (C57) mice were infused with protein stabilized sonoactive microstructures (Optison), and 250 micrograms of nucleic acids encoding a luciferase reporter gene coupled to a CAG promoter (pDNA-luc) via a surgically implanted jugular vein catheter (JVC) in 50 microliters of PBS, and were split into 3 experimental groups receiving a single, two, or three dosages and sonoporation treatments. Three additional wild type (C57) mice were infused with 250 micrograms of nucleic acids encoding a luciferase reporter gene coupled to a CAG promoter (pDNA-luc) via a surgically implanted jugular vein catheter (JVC) in 50 microliters of PBS and were administered no sonoactive microstructures but did receive ultrasound. One additional mouse was injected with 150 μL of phospholipid stabilized Sonazoid sonoactive microstructures and 250 μg pDNA-luc in 50 μL in PBS for a single dose and a single treatment. A first experimental group received a single application of ultrasound acoustic energy and one dose of protein stabilized sonoactive microstructures, a second experimental group received two applications of ultrasound acoustic energy and two doses of protein stabilized sonoactive microstructures, a third experimental group received three applications of ultrasound acoustic energy and three doses of protein stabilized sonoactive microstructures, the fourth experimental group received a single application of ultrasound acoustic energy and no sonoactive microstructures, and a single mouse in a fifth experimental group received a single application of ultrasound acoustic energy and one dose of phospholipid stabilized sonoactive microstructures.

In each treatment session, ultrasound was applied to the liver of each mouse with a GE LOGIQ e10 ultrasound system equipped with a C1-6 probe, and use of the “ELASTO” software, Shear Wave. The shear wave ultrasound acoustic radiation force protocol was applied at an ultrasound intensity of 187.9 mW/cm² (I_{SPTA}) (spatial-peak temporal average intensity), at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%. Ultrasound energy was further applied to generate a B-mode ultrasound image at a mechanical index of 1.4, and a frequency of 2.5 MHz. A rapid loss of ultrasound contrast (due to inertial cavitation of the sonoactive agent) occurred following the application of the acoustic radiation force.

SWE ULTRASOUND PARAMETERS GE LOGIQ
e10 C1-6 probe with ELASTO software

| MI | 1.4 | |
|---|---|---|
| TIs | 0.3 | |
| Frame Rate | 55 | |
| CHI | | |
| Frequency | 2.5 | MHz |
| Gain | 44 | |
| AO % | 100 | |
| E | | |
| Gain | 55 | |
| T | 8 | |
| SVD | 3.5 | |
| PO % | 100% | |
| TO | 100% | |
| F | 50-400 | Hz |

| | |
|---|---|
| Penetration | — |
| ISPTA | 187.9 |

Mice 1-3 were administered 150 μL of protein stabilized Optison sonoactive microstructures and 250 μg pDNA-luc in 50 microliters of PBS via the JVC, and an acoustic radiation force protocol was applied for 10 seconds, followed by 20 seconds with no application of ultrasound, followed again by 10 seconds of an acoustic radiation force protocol, for a total time of 40 seconds of acoustic radiation force protocol applied. Mice 4-6 each were administered two sonoporation treatments four hours apart each treatment comprising: administration of 150 μL of protein stabilized Optison sonoactive microstructures and 250 μg pDNA-luc in 50 microliters of PBS via the JVC, and an acoustic radiation force protocol applied for 10 seconds, followed by 20 seconds with no application of ultrasound, followed again by 10 seconds of an acoustic radiation force protocol, for a total time of 40 seconds of acoustic radiation force protocol applied. Mice 7-9 each were administered three sonoporation treatments four hours apart each treatment comprising: administration of 150 μL of protein stabilized Optison sonoactive microstructures and 250 μg pDNA-luc in 50 microliters of PB via the JVC, and an acoustic radiation force protocol applied for 10 seconds, followed by 20 seconds with no application of ultrasound, followed again by 10 seconds of an acoustic radiation force protocol, for a total time of 40 seconds of acoustic radiation force protocol applied. Mice 10-12 were administered 250 μg pDNA-luc in 50 microliters of PBS via the JVC, and an acoustic radiation force protocol was applied for 10 seconds, followed by 20 seconds with no application of ultrasound, followed again by 10 seconds of an acoustic radiation force protocol, for a total time of 40 seconds of acoustic radiation force protocol applied. Mouse 13 was administered 150 μL of phospholipid stabilized Sonazoid sonoactive microstructures and 250 μg pDNA-luc in 50 microliters of PB via the JVC, and an acoustic radiation force protocol was applied for 10 seconds, followed by 20 seconds with no application of ultrasound, followed again by 10 seconds of an acoustic radiation force protocol, for a total time of 40 seconds of acoustic radiation force protocol applied.

The shear wave ultrasound acoustic radiation force protocol was applied at an ultrasound intensity of 187.9 mW/cm² (I_{SPTA}) (spatial-peak temporal average intensity), at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%. Ultrasound energy was applied using a constant imaging technique applying ultrasound and generating a B-mode ultrasound image at a mechanical index of 0.09, and a frequency of 2.5 MHz.

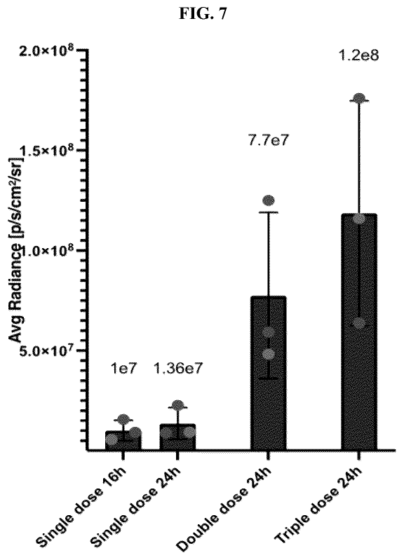

Following the procedure, each mouse underwent IVIS imaging receiving isoflurane sedation and a D-Luciferin injections to detect luciferase within the targeted organ (liver). IVIS images were collected at 24 hours, and revealed a high signal over the abdomen in the mice treated with the shear wave ultrasound acoustic radiation force protocol, with mice having undergone ketamine sedation exhibiting increased luciferase signal. The experimental groups and results are summarized below, and in FIG. 7.

| | | Total | IVIS |
|---|---|---|---|
| | Doses and Treatments | time of | (p/sec/cm²/sr) at 24 |
| Mice | Administered | ARF (sec) | hours (average) |
| 1-3 (C57) | Single | 40 | 1E7 |
| 4-6 (C57) | Double | 40 | 1.36E7 |
| 7-9 (C57) | Triple | 40 | 7.7E7 |
| 10-12 (C57) | Single - no microbubbles | 40 | 1E3 (background |
| | | | signal) |
| 13 (C57) | Single - phospholipid | 40 | 3.72E6 |
| | stabilized sonoactive | | |
| | microstructures | | |

### Example VI.: Sonoporation Using Ultrasound Inducing Shear Waves in Repeated Treatments Delivering FVIII to the Murine Liver

In this example, application of sonoporation in multiple treatments utilizing ultrasound to apply an acoustic radiation force and induce shear waves in a mouse liver to delivery FVIII was investigated. Three (RAG2) mice are infused with protein stabilized sonoactive microstructures (Optison), and 50 micrograms of nucleic acids encoding a human factor VIII transgene (NP-ApoE-AAT-FVIIIv3) via a surgically implanted jugular vein catheter (JVC) in 50 microliters of PBS. Each mouse receives a total of three doses of the nucleic acid payload, doses of protein stabilized sonoactive microstructures infused with the nucleic acid payload and three applications of ultrasound acoustic energy. Each dose of the nucleic acid payload, protein stabilized sonoactive microstructures, and ultrasound acoustic energy are administered in a treatment session, with the second treatment session administered 48 hours following the first treatment session, and the third treatment session administered 48 hours after the second treatment session.

In each treatment session, ultrasound was applied to the liver of each mouse were performed with a GE LOGIQ e10 ultrasound system equipped with a C1-6 probe, and use of the “ELASTO” software, Shear Wave. The shear wave ultrasound acoustic radiation force protocol was applied using ultrasound acoustic energy at an ultrasound intensity of 187.9 mW/cm² (I_{SPTA}) (spatial-peak temporal average intensity), at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%, with parameters more fully detailed below. B-mode ultrasound energy was also applied generated a B-mode ultrasound image at a mechanical index of 0.09, and a frequency of 2.5 MHz, to confirm the presence of the sonoactive agent in the target liver tissue. A rapid loss of ultrasound contrast (due to inertial cavitation of the sonoactive agent) occurred following the application of the acoustic radiation force.

SWE ULTRASOUND PARAMETERS GE LOGIQ
e10 C1-6 probe with ELASTO software

| MI | 1.4 | |
|---|---|---|
| TIs | 0.3 | |
| Frame Rate | 55 | |
| CHI | | |
| Frequency | 2.5 | MHz |
| Gain | 44 | |
| AO % | 100 | |
| E | | |
| Gain | 55 | |
| T | 8 | |
| SVD | 3.5 | |
| PO % | 100% | |
| TO | 100% | |
| F | 50-400 | Hz |

| | |
|---|---|
| Penetration | — |
| ISPTA | 187.9 |

Mice are administered the sonoactive microstructures and the nucleic acids encoding human FVIII in 50 microliters of PBS via the JVC, and the acoustic radiation force protocol was applied for 10 seconds, followed by 20 seconds with no application of ultrasound, followed again by 10 seconds of an acoustic radiation force protocol, for a total time of 40 seconds of acoustic radiation force application.

Following the procedure, each retroorbital (RO) bleed samples are collected at 8, 12, 19, 26, and 33 days following the final treatment are assessed for FVIII content μs by immunoassay (MESO SCALE DIAGNOSTICS, LLC). Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.92 IU/ml to 0.01 IU/ml. 2-fold diluted samples and standards were added to the wells in 96-well plate. Incubated 2 hours at room temperature and washed 3 times. The detection was performed by incubating samples with GMA-8023 antibody during 2 hours following triple wash. Signal was developed by Sulfo-TAG and detected by MSD machine.

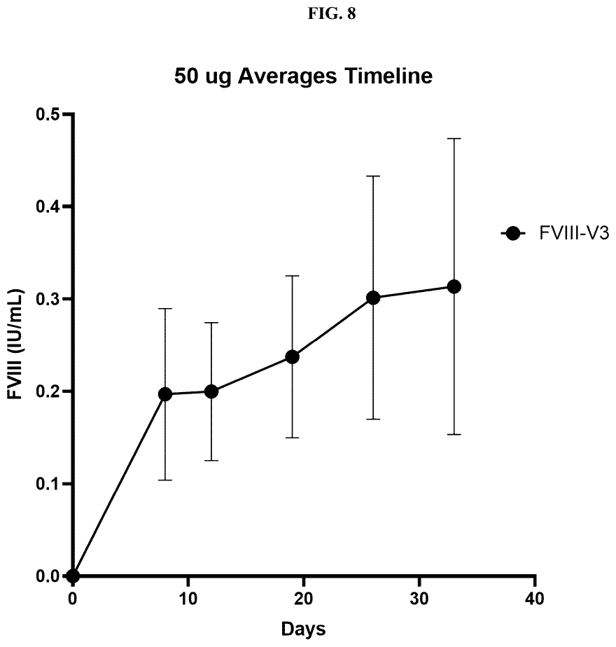

Results are shown in FIG. 8 in which it is shown that FVIII levis of about 0.2 IU/mL are achieved at day 8, and increase to about 0.3 IU/ML at day 33, significantly exceeding the therapeutic threshold of FVIII of 0.05 IU/mL.

### Example VII.: Sonoporation Using Ultrasound Inducing Shear Waves Compared to B-Mode Ultrasound in a Non-Human Primate Kidney Model

In this example, application of sonoporation in a single treatment utilizing ultrasound to apply an acoustic radiation force and induce shear waves in a NHP kidney to deliver green fluorescent protein (GFP) was investigated. In a single male cynomolgus macaque (cyno), the cyno was infused with phospholipid stabilized sonoactive microstructures (Sonazoid), and 10 milligrams of nucleic acids encoding a GFP under the influence of a CAG promoter (CAG-GFP) via an intravenous infusion into the saphenous vein. The left kidney was treated using ultrasound to apply an acoustic radiation force and induce shear waves, while the right kidney was treated with B-mode ultrasound. The subject was administered 10 mg of the CAG-GFP nucleic acid total and applied apply an acoustic radiation force to the left kidney and B-mode ultrasound in a single treatment session, with a total of 4 treatment session being administered on days 1, 5, 7, and 13. Ultrasound energy was applied using a M5Sc probe on a GE LOGIQ E10 system in research mode for the B-mode ultrasound application The ultrasound parameters are described below.

The B-mode ultrasound was applied at focal depth setting was set to 7 cm, and the zoom to 0. Ultrasound was delivered continuously and alternated between a low mechanical index (MI) value of 0.09 and a high MI value of 2.3. The low MI pulse duration ranged from 0.63-0.93 microseconds and was applied at a frequence of 2.07-2.9 MHz, depending on the contrast imaging frequency mode selected. The high MI ultrasound was delivered approximately 20 seconds after administration of the IV infusion following visualization of the sonoactive agent with the low MI ultrasound. The high MI pulse duration was 2.28 microseconds and was applied at a frequency of 1.64 MHz. Upon disruption of the sonoactive agent with application the high MI ultrasound, serial bolus injections of approximately 1.0-2.0 mL of sonoactive microstructure and DNA solution were administered to each subject about every 30 seconds until the 10 mL of DNA and microbubble solution was fully administered for the target organ, with the total treatment time occurring over about 10 minutes. 5 mg of the CAG-GFP nucleic acid were administered in this first treatment of the first kidney.

The acoustic radiation force ultrasound was applied to the kidney of the cyno with the same GE LOGIQ e10 ultrasound system, but equipped with a C1-6 probe operating using the “ELASTO” software, Shear Wave mode. The ARF to the second kidney was applied immediately following the application to the B-mode ultrasound to the first kidney. To begin the treatment of the second kidney, B-mode ultrasound energy was also applied generated a B-mode ultrasound image at a mechanical index of 0.09, and a frequency of 2.5 MHz, to confirm the presence of the sonoactive agent in the target kidney tissue in an initial 15-30 second applications before administering the ARF. The shear wave ultrasound acoustic radiation force protocol was applied using ultrasound acoustic energy at an ultrasound intensity of at least 180 mW/cm² (I_{SPTA}) (spatial-peak temporal average intensity), at a focal depth of 5-6 cm, at a frequency of 2.5 MHz, at a mechanical index of 2.1, at a pulse length of about 600 us, a pulse repetition frequency of about 0.9 Hz, a pulse repetition period of about 1.1 s and a duty cycle of about 0.05%, and tracking output reduced to 0.1%. Upon disruption of the sonoactive agent with application the ARF ultrasound, serial bolus injections of approximately 1.0-2.0 mL of sonoactive microstructure and DNA solution were re-administered to the subject about every 30 seconds, and the acoustic radiation force ultrasound was applied until the 10 mL of DNA and microbubble solution was fully administered for the target organ, with the total treatment time occurring over about 10 minutes. 5 mg of the CAG-GFP nucleic acid were administered in this second treatment of the second kidney. A rapid loss of ultrasound contrast (due to inertial cavitation of the sonoactive agent) occurred following the application of the acoustic radiation force.

On day 18 of the study, necropsy was performed, and samples the treated organs were collected. Kidney samples were bisected and cut into cross section 1.5 cm thick to capture all layers. Samples from the center section were placed into neutral 10% formalin for ddPCR assay and transferred to 70% EtOH 20 hours post-fixation.

Samples were evaluated using digital droplet polymer chain reaction (ddPCR) to assess copy number per diploid genome (CN/DG) levels of DNA payload delivered to the cells. The procedure began by preparing the Master Mix (MM) for each reaction. First, the ddPCR Supermix for probes (no dUTP) was thawed and vortexed for at least 30 seconds. For each primer/probe set, 11 μL ddPCR Supermix, 1.1 μL primer/probe mix, 0.275 μL HindIII enzyme, and water were combined to bring the final reaction volume to 18 μL. For single-plex reactions, 5.626 μL of water was added, while for duplex reactions, 4.525 μL was added. The Master Mix was calculated with a correction factor of 1.1 to account for pipetting error. After preparation, 18 μL of the Master Mix was dispensed into individual wells of a 96-well PCR plate, which was then set aside at room temperature. DNA samples were diluted using nuclease-free water to the desired concentration, and 4 μL of the diluted genomic DNA (gDNA) or ddFHO (for negative controls) was added to each well according to the plate map. The plate was then sealed with a pierceable foil seal, ensuring the red stripe faced up at the top, and vortexed for 2 minutes at room temperature. Following this, the plate was spun down at 100-300 rcf for 1 minute to remove bubbles. The sealed plate was placed in the Automated Droplet Generator (ADG), ensuring that the destination plate on a cooling block, DG32 cartridges, ddPCR tips (with lids removed), and an empty waste container were properly positioned. The droplet generation process was initiated, and the corresponding ddPCR program was selected on the thermal cycler, which was paused while waiting for the droplet generation to finish. Once the droplet generation was completed, the 96-well plate was removed from the cooling block, resealed with a pierceable foil seal, and transferred to the thermal cycler, where the cycling process was resumed. The cooling block was returned to −20° C. in an upside-down position for future use. Thermal cycling was performed based on the ddPCR program, which typically included steps such as denaturation, annealing, and extension, although specific conditions depended on the primer/probe set used. After thermal cycling, the plate was transferred to the Droplet Reader. In the ddPCR software, the appropriate Supermix and fluorophores (e.g., FAM, VIC, HEX) were selected before initiating the droplet reading process. Once the droplet reading was completed, the results provided the quantification of plasmid DNA copy number per diploid genome, as well as its relative abundance to reference genes. The software generated data based on the fluorescence signals detected in the droplets, enabling accurate measurement of the target gene copy numbers.

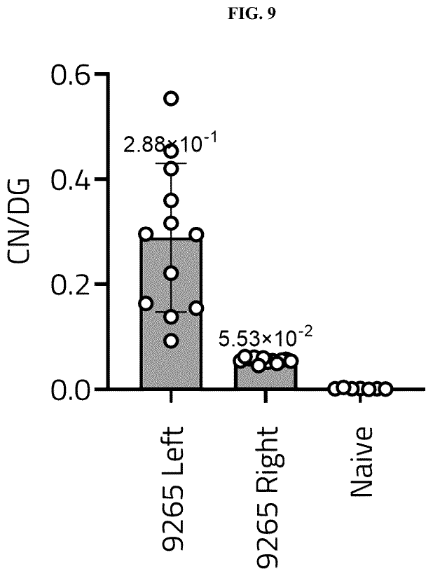

Results are shown in FIG. 9, where it is observed that the left kidney receiving application of the acoustic radiation force ultrasound exhibited an average copy number per diploid genome (CN/DG) of about 2.88 e-1 CN/DG, while the right kidney receiving application of the 5.53 e-2 CN/DG, representing approximately a 5.3 fold (e.g., 5.3×) increase in gene expression as measured by copy number in the left kidney receiving application of the radiation force ultrasound over the right kidney treated with B-mode ultrasound.

Results in CN/DG can be converted to copy number per nanogram of DNA (CN/ng) by use of the conversion factor of 0.006 ng of DNA per diploid genome. Applying this conversion factor, an average copy number per ng of genomic DNA (CN/ng) of about 46.6 CN/ng can be obtained with respect to the receiving application of the acoustic radiation force ultrasound.

### Example VIII.: Sonoporation Using Ultrasound Inducing Shear Waves, with CFT Biodistribution Assessment in the Murine Liver

In this example, application of sonoporation in multiple treatments utilizing ultrasound to apply an acoustic radiation force and induce shear waves in a mouse liver to deliver a tdTomato reporter gene was investigated, and biodistribution of the reporter in the murine liver was assessed using cryofluorescence tomography (CFT). One group of (RAG2) mice are infused with protein stabilized sonoactive microstructures (Optison), and 100 micrograms of nucleic acids encoding a tdTomato reporter gene under the influence of a CAG promoter (CAG-tdTomato) via a surgically implanted jugular vein catheter (JVC) in 50 microliters of PBS. Each mouse receives a total of three doses of the nucleic acid payload, doses of protein stabilized sonoactive microstructures infused with the nucleic acid payload and three applications of ultrasound acoustic energy. Each dose of the nucleic acid payload, protein stabilized sonoactive microstructures, and ultrasound acoustic energy are administered in a treatment session, with the second treatment session administered 24 hours following the first treatment session, and the third treatment session administered 24 hours after the second treatment session. A single untreated RAG2 mouse served as a negative control.

In each treatment session, ultrasound was applied to the liver of each mouse were performed with a GE LOGIQ e10 ultrasound system equipped with a C1-6 probe, and use of the “ELASTO” software, Shear Wave. The shear wave ultrasound acoustic radiation force protocol was applied using ultrasound acoustic energy at an ultrasound intensity of 187.9 mW/cm² (I_{SPTA}) (spatial-peak temporal average intensity), at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%, with parameters more fully detailed below. B-mode ultrasound energy was also applied generated a B-mode ultrasound image at a mechanical index of 0.09, and a frequency of 2.5 MHz, to confirm the presence of the sonoactive agent in the target liver tissue. A rapid loss of ultrasound contrast (due to inertial cavitation of the sonoactive agent) occurred following the application of the acoustic radiation force.

SWE ULTRASOUND PARAMETERS GE LOGIQ
e10 C1-6 probe with ELASTO software

| MI | 1.4 | |
|---|---|---|
| TIs | 0.3 | |
| Frame Rate | 55 | |
| CHI | | |
| Frequency | 2.5 | MHz |
| Gain | 44 | |
| AO % | 100 | |
| E | | |
| Gain | 55 | |
| T | 8 | |
| SVD | 3.5 | |
| PO % | 100% | |
| TO | 100% | |
| F | 50-400 | Hz |

| | |
|---|---|
| Penetration | — |
| ISPTA | 187.9 |

Mice are administered the sonoactive microstructures and the nucleic acids encoding in PBS via the JVC, and the acoustic radiation force protocol was applied for 10 seconds, followed by 20 seconds with no application of ultrasound, followed again by 10 seconds of an acoustic radiation force protocol, for a total time of 40 seconds of acoustic radiation force application.

1 week following the final treatment, necropsies are performed on a treated subject and the livers were snap frozen in liquid N₂ and shipped over dry ice for cryofluorescence tomography (CFT) analysis. In brief, tissues were embedded in OCT compound in 1 sample block. The sample block was serially sectioned at 30 μm in a cryomacrotome along with sequential imaging of the block face at each sectioning plane using Xerra Controller software (Emit Imaging). High resolution images with white light and test article compatible fluorescent channels were acquired by Emit Imaging using the CFT Xerra system and Xerra Controller software with the following configurations: White Light Channel: RGB, Fluorescent Channel: excitation: 555 nm, emission: 586 nm. Images were reconstructed and processed in the Emit Xerra Reconstruction Software. MHD files (Metalmage MetaHeader files) were generated for each tissue for WL and FL images, using fluorescent images acquired with autoexposure of 500 ms. Fluorescent images were co-registered to WL images with VivoQuant™ software. Reconstructed and processed images were used to generate Maximum Intensity Projection images (MIPs) and flythrough movies. Values are expressed in Normalized Corrected Counts (NCC).

Results are shown in FIGS. 10A-10G in which expression of the tdTomato reporter is visualized throughout the organ as a measure of biodistribution of the nucleic acid delivery and transfection to cells within the liver. FIGS. 10A-10G are images taken from a time lapse video which shows a treated liver undergoing a single 360 degree rotation about an axis parallel a plane of the figure, the axis running parallel to long edge of the paper when viewed in portrait orientation. FIG. 10A is an initial image, and FIG. 10G is a final image of the time lapsed rotation. FIGS. 10B-10G each represent approximately a 30 degree rotation relative to the prior image (e.g., FIG. 10B to FIG. 10C). In FIGS. 10A-10G, it is shown the treated liver exhibits many individual puncta indicating expression of the tdTomato reporter, with fluorescence ranging from red (˜3000 NCC) to orange (˜4000 NCC) across substantially the entire treated organ, and further yellow (˜5000 NCC) in clustered regions of puncta. In each panel of the treated liver images in FIGS. 10A-10G, it is observed that the individual puncta indicating expression of the tdTomato reporter form the shape of a murine liver, visualizing the biodistribution of the tdTomato reporter across the organ, in which it is observed that all regions of the treated murine liver exhibit expression of the tdTomato reporter throughout the organ.

In parallel, samples from the fourth experimental subject were evaluated using digital droplet polymer chain reaction (ddPCR) to assess copy number per diploid genome (CN/DG) levels of DNA payload delivered to the cell in the organ. The liver of each subject is split into a right and a left section. The procedure began by preparing the Master Mix (MM) for each reaction. First, the ddPCR Supermix for probes (no dUTP) was thawed and vortexed for at least 30 seconds. For each primer/probe set, 11 μL ddPCR Supermix, 1.1 μL primer/probe mix, 0.275 μL HindIII enzyme, and water were combined to bring the final reaction volume to 18 μL. For single-plex reactions, 5.626 μL of water was added, while for duplex reactions, 4.525 μL was added. The Master Mix was calculated with a correction factor of 1.1 to account for pipetting error. After preparation, 18 μL of the Master Mix was dispensed into individual wells of a 96-well PCR plate, which was then set aside at room temperature. DNA samples were diluted using nuclease-free water to the desired concentration, and 4 μL of the diluted genomic DNA (gDNA) or ddH₂O (for negative controls) was added to each well according to the plate map. The plate was then sealed with a pierceable foil seal, ensuring the red stripe faced up at the top, and vortexed for 2 minutes at room temperature. Following this, the plate was spun down at 100-300 rcf for 1 minute to remove bubbles. The sealed plate was placed in the Automated Droplet Generator (ADG), ensuring that the destination plate on a cooling block, DG32 cartridges, ddPCR tips (with lids removed), and an empty waste container were properly positioned. The droplet generation process was initiated, and the corresponding ddPCR program was selected on the thermal cycler, which was paused while waiting for the droplet generation to finish. Once the droplet generation was completed, the 96-well plate was removed from the cooling block, resealed with a pierceable foil seal, and transferred to the thermal cycler, where the cycling process was resumed. The cooling block was returned to −20° C. in an upside-down position for future use. Thermal cycling was performed based on the ddPCR program, which typically included steps such as denaturation, annealing, and extension, although specific conditions depended on the primer/probe set used. After thermal cycling, the plate was transferred to the Droplet Reader. In the ddPCR software, the appropriate Supermix and fluorophores (e.g., FAM, VIC, HEX) were selected before initiating the droplet reading process. Once the droplet reading was completed, the results provided the quantification of DNA copy number per diploid genome, as well as its relative abundance to reference genes. The software generated data based on the fluorescence signals detected in the droplets, enabling accurate measurement of the target gene copy numbers.

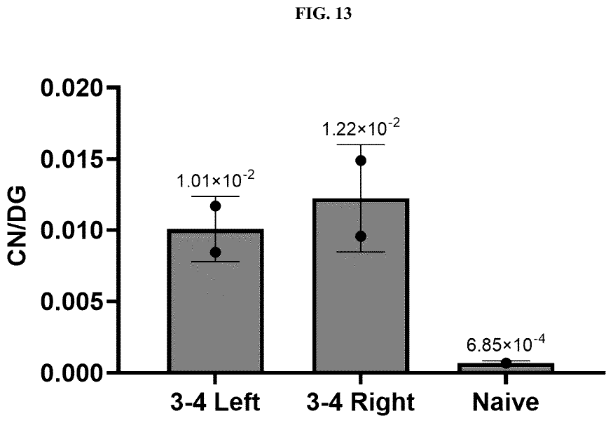

Results are shown in FIG. 13, where it is observed that the right and left sections of a subject liver treated with the acoustic radiation force ultrasound exhibited an average copy number per diploid genome (CN/DG) of about 1.22 e-2 CN/DG and 1.01 e-2 respectively.

Results in CN/DG can be converted to copy number per nanogram of DNA (CN/ng) by use of the conversion factor of 0.006 ng of DNA per diploid genome. Applying this conversion factor, an average copy number per ng of genomic DNA (CN/ng) of about 2 CN/ng and 1.67 CN/ng can be obtained with respect to the right and left sections of the fourth subject liver treated with the acoustic radiation force.

### Example IX.: Sonoporation Using Ultrasound Inducing Shear Waves in Repeated Treatments Assessed for Durability of Gene Expression in the Murine Liver

In this example, application of sonoporation in multiple treatments utilizing ultrasound to apply an acoustic radiation force and induce shear waves in a mouse liver was investigated for durability of gene expression over a 27 week period. Two groups of 4 RAG2 each mice were infused with protein stabilized sonoactive microstructures (Optison), and 50 micrograms of nucleic acids encoding a luciferase reporter gene coupled to a CAG promoter (pDNA-luc) via a surgically implanted jugular vein catheter (JVC) in 50 microliters of PBS, and were split into two experimental groups, each group receiving a total of three sonoporation treatments at either 24 or 48 hour intervals between treatments.

In each treatment session, ultrasound was applied to the liver of each mouse with a GE LOGIQ e10 ultrasound system equipped with a C1-6 probe, and use of the “ELASTO” software, Shear Wave. The shear wave ultrasound acoustic radiation force protocol was applied at an ultrasound intensity of 187.9 mW/cm² (I_{SPTA}) (spatial-peak temporal average intensity), at a mechanical index of 1.4, and a frequency of 2.5 MHz. Ultrasound energy was further applied to generate a B-mode ultrasound image at a mechanical index of 0.09, and a frequency of 2.5 MHz, at a pulse length of about 600 us, a pulse repetition frequency of about 1.7 Hz, a pulse repetition period of about 0.58 s and a duty cycle of about 0.1%. A rapid loss of ultrasound contrast (due to inertial cavitation of the sonoactive agent) occurred following the application of the acoustic radiation force.

SWE ULTRASOUND PARAMETERS GE LOGIQ
e10 C1-6 probe with ELASTO software

| MI | 1.4 | |
|---|---|---|
| TIs | 0.3 | |
| Frame Rate | 55 | |
| CHI | | |
| Frequency | 2.5 | MHz |
| Gain | 44 | |
| AO % | 100 | |
| E | | |
| Gain | 55 | |
| T | 8 | |
| SVD | 3.5 | |
| PO % | 100% | |
| TO | 100% | |
| F | 50-400 | Hz |

| | |
|---|---|
| Penetration | — |
| ISPTA | 187.9 |

Both groups of mice were administered sonoactive microstructures and the DNA payload in PBS via the JVC, and an acoustic radiation force protocol was applied for 10 seconds, followed by 20 seconds with no application of ultrasound, followed again by 10 seconds of an acoustic radiation force protocol, for a total time of 40 seconds of acoustic radiation force protocol applied. Group 1 mice received identical treatments on days 1, 3, and 5. Group 2 mice received identical treatments on days 1, 2, and 3.

Following the procedure, each mouse underwent IVIS imaging receiving isoflurane sedation and a D-Luciferin injections to detect luciferase within the targeted organ (liver). IVIS images were collected at 24 and 72 hours following the second and third treatments, and then at one-half or one-week intervals for 27 weeks following the final treatment.

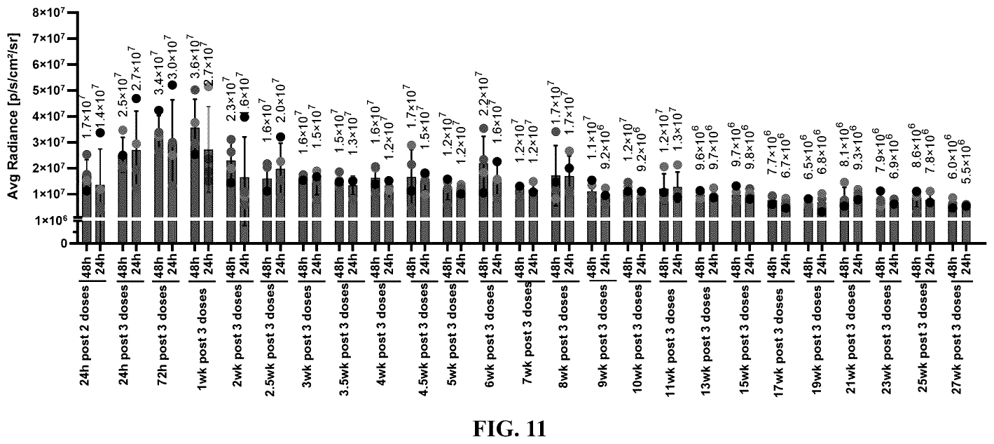

Results are shown in FIG. 11 in which it is shown that average radiance levels of about 3e7 to 3.4e7 p/s/cm2/sr are observed at 72 hours following the final treatment, and are maintained at levels of at least 1e7 p/s/cm2/sr through 8 weeks, and are maintained at levels of 5.4e6 to 1e7 p/s/cm2/sr for the remainder of the study through 27 weeks post final treatment. These results show that high levels of gene expression can be achieved and maintained for extended periods using ultrasound to apply an acoustic radiation force and induce shear waves in target tissue.

### Example X.: Sonoporation Using Ultrasound Inducing Shear Waves in Repeated Treatments in the Murine Heart

In this example, application of sonoporation in multiple treatments utilizing ultrasound to apply an acoustic radiation force and induce shear waves in the murine heart was investigated. 3 RAG2 mice were infused with phospholipid stabilized sonoactive microstructures (Sonazoid), and 250 micrograms of nucleic acids encoding a GFP reporter gene coupled to a CAG promoter (pDNA-GFP) via a surgically implanted jugular vein catheter (JVC) in 50 microliters of PBS. A control group was administered only the 250 micrograms of nucleic acids encoding a GFP reporter gene coupled to a CAG promoter (pDNA-GFP) and were not applied any ultrasound in each treatment session.

For the experimental group receiving ultrasound, in each treatment session, ultrasound was applied to the liver of each mouse with a GE LOGIQ e10 ultrasound system equipped with a L8-18 probe, and use of the “ELASTO” software, Shear Wave. The shear wave ultrasound acoustic radiation force protocol was applied at an ultrasound intensity of about 250 mW/cm² (I_{SPTA}) (spatial-peak temporal average intensity), at a mechanical index of 0.8, and a frequency of 18 MHz at a pulse length of about 200 us, a pulse repetition frequency of about 0.92 Hz, a pulse repetition period of about 1.08 s, and a duty cycle of about 0.18%. Focal depth was set to 1.9 cm. Ultrasound energy was further applied to generate a B-mode ultrasound image at a mechanical index of 0.09, and a frequency of 18 MHz. A rapid loss of ultrasound contrast (due to inertial cavitation of the sonoactive agent) occurred following the application of the acoustic radiation force.

Mice in Group 1 were administered sonoactive microstructures and the DNA payload in PBS via the JVC, and an ultrasound applying an acoustic radiation force was applied for 40 seconds continuously. Mice in Group 1 were re-treated on days 3 and 5 following the initial treatment.

Following the procedure, each mouse underwent IVIS imaging receiving isoflurane sedation and a D-Luciferin injections to detect luciferase within the targeted organ (liver). IVIS images were collected at 96 hours, and revealed a high signal over the chest region in the mice treated with the shear wave ultrasound acoustic radiation force protocol.

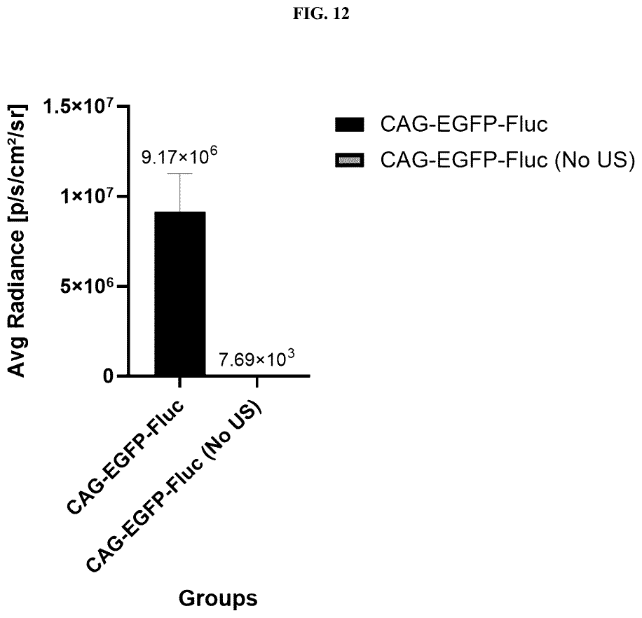

Results are shown in FIG. 12, in which the treated mice exhibit an average radiance of about 9.17 e6, while untreated mice exhibit a radiance level of about 7.69 e3 consistent with background levels.

### Example XI: Sonoporation Using Ultrasound Inducing Shear Waves in the NHP Liver

In this example, application of sonoporation in a single treatment utilizing ultrasound to apply an acoustic radiation force and induce shear waves in a NHP liver to deliver green fluorescent protein (GFP) is investigated. In a single male cynomolgus macaque (cyno), the cyno is infused with phospholipid stabilized sonoactive microstructures (Sonazoid), and 10 milligrams of nucleic acids encoding a GFP under the influence of a CAG promoter (CAG-GFP) via an intravenous infusion into the saphenous vein. The liver was treated using ultrasound to apply an acoustic radiation force and induce shear waves. The cyno was administered 10 mg of the CAG-GFP nucleic acid total and applied apply an acoustic radiation force to the liver. Ultrasound energy was applied using a C-16 probe on a GE LOGIQ E10 system in research mode for the acoustic radiation force ultrasound application. The ultrasound parameters are described below.

The acoustic radiation force ultrasound was applied to the liver of the cyno with the same GE LOGIQ e10 ultrasound system, but equipped with a C1-6 probe operating using the “ELASTO” software, Shear Wave mode. First, B-mode ultrasound energy is applied to generate a B-mode ultrasound image at a mechanical index of 0.09, and a frequency of about 2.5 MHz to confirm the presence of the sonoactive agent in the target liver tissue in an initial 15-30 second applications before administering the ARF. The shear wave ultrasound acoustic radiation force protocol is then applied using ultrasound acoustic energy at an ultrasound intensity of about 500 mW/cm² (I_{SPTA}) (spatial-peak temporal average intensity), a frequency of about 2.3 MHz. Upon disruption of the sonoactive agent with application the ARF ultrasound, serial bolus injections of approximately 1.0-2.0 mL of sonoactive microstructure and DNA solution were re-administered to the subject about every 30 seconds, and the acoustic radiation force ultrasound was applied until the 10 mL of DNA and microbubble solution was fully administered for the target organ, with the total treatment time occurring over about 10 minutes. A rapid loss of ultrasound contrast (due to inertial cavitation of the sonoactive agent) occurred following the application of the acoustic radiation force.

3 or more days following completion of the study, necropsy was performed, and the treated organs were collected. The treated organs are imaged using an IVIS system to assess the nucleic acid expression of the reporter as measured by the average radiance of the organ. The treated organs exhibit average radiance consistent with the elevated levels of expression observed in the murine liver treated with the ARF protocol.

### Example XII: Sonoporation Using Ultrasound Inducing Shear Waves in the Murine Liver with Tissue Histology Assessment

In this example, application of sonoporation in multiple treatments utilizing ultrasound to apply an acoustic radiation force and induce shear waves in a mouse liver to deliver a plasmid encoding human clotting FVIII (hFVIII) was investigated, and impact on the treated murine liver tissue was assessed using hematoxylin and eosin (H&E) staining for impact to tissue morphology.

Three groups of four (RAG2) mice are infused with protein stabilized sonoactive microstructures (Optison), and either 50, or 250 micrograms of nucleic acids encoding a hFVIII under the influence of an APOE-AAT promoter (APOE-AAT-hFVIII) via a surgically implanted jugular vein catheter (JVC) in 50 microliters of PBS. Each mouse receives a single of the nucleic acid payload, doses of protein stabilized sonoactive microstructures infused with the nucleic acid payload and a single application of ultrasound acoustic energy. A single untreated RAG2 mouse served as a negative control. A group of four RAG2 mice are untreated and serve as a control.

In the treatment session, ultrasound was applied to the liver of each mouse were performed with a GE LOGIQ e10 ultrasound system equipped with a C1-6 probe, and use of the “ELASTO” software, Shear Wave. The shear wave ultrasound acoustic radiation force protocol was applied using ultrasound acoustic energy at an ultrasound intensity of 187.9 mW/cm² (I_{SPTA}) (spatial-peak temporal average intensity), at a mechanical index of 1.4, and a frequency of 2.5 MHz, at a pulse length of about 600 us, a pulse repetition frequency of about 1.7 Hz, a pulse repetition period of about 0.58 s and a duty cycle of about 0.1%, with parameters more fully detailed below. B-mode ultrasound energy was also applied generated a B-mode ultrasound image at a mechanical index of 0.09, and a frequency of 2.5 MHz, to confirm the presence of the sonoactive agent in the target liver tissue. A rapid loss of ultrasound contrast (due to inertial cavitation of the sonoactive agent) occurred following the application of the acoustic radiation force.

SWE ULTRASOUND PARAMETERS GE LOGIQ
e10 C1-6 probe with ELASTO software

| MI | 1.4 | |
|---|---|---|
| TIs | 0.3 | |
| Frame Rate | 55 | |
| CHI | | |
| Frequency | 2.5 | MHz |
| Gain | 44 | |
| AO % | 100 | |
| E | | |
| Gain | 55 | |
| T | 8 | |
| SVD | 3.5 | |
| PO % | 100% | |
| TO | 100% | |
| F | 50-400 | Hz |

| | |
|---|---|
| Penetration | — |
| ISPTA | 187.9 |

Mice are administered the sonoactive microstructures and the nucleic acids encoding in PBS via the JVC, and the acoustic radiation force protocol was applied for 10 seconds, followed by 20 seconds with no application of ultrasound, followed again by 10 seconds of an acoustic radiation force protocol, for a total time of 40 seconds of acoustic radiation force application.

48 following the treatment, necropsies are performed on one mice from each group of treated subjects and the control subject, and the livers were embedded in neutral buffered formalin (NBF) for 20 hours to preserve cellular and structural integrity, then the NBF was discarded, and replaced with 70% ethanol to transition the samples into a storage-friendly medium, preventing over-fixation and promoting dehydration. The liver tissues were subsequently processed for paraffin embedding, involving sequential dehydration in increasing ethanol concentrations, clearing in xylene, and infiltration with molten paraffin wax. The tissues were embedded into paraffin blocks, sectioned at 4-6 m thickness using a microtome, and mounted on glass slides pre-treated to ensure tissue adherence. The sections were dried and prepared for staining.

The slides were placed in a slide holder and attached to an auto-stainer, and the start button was engaged to initiate the automated H&E staining process. The protocol began with deparaffinization and rehydration: the sections were immersed in xylene for three cycles of three minutes each, followed by two cycles of three minutes in 100% reagent alcohol, one three-minute cycle in 95% reagent alcohol, one three-minute cycle in 80% reagent alcohol, and finally rinsed in water to complete rehydration.

Hematoxylin staining was performed next, with the slides incubated in Richard Allan Hematoxylin for four minutes. Following the staining, the slides were rinsed thoroughly in water, dipped for 30 seconds in 4% acetic acid, and rinsed again in tap water. Differentiation was achieved with 10 dips in 0.3% ammonia water, followed by a final rinse in tap water to complete the nuclear staining phase.

For eosin staining and dehydration, the sections were immersed for 30 seconds in Richard Allan Eosin with phloxine to stain cytoplasmic and extracellular components. Dehydration steps included 10 dips in 95% ethanol, followed by two one-minute cycles in 100% reagent alcohol, and finally three one-minute cycles in xylene to clear the sections. The stained slides were removed from the racks, air-dried, and coverslipped with a resin-based mounting medium to preserve the sections and facilitate microscopic examination. This standardized protocol yielded high-quality stained sections suitable for histopathological analysis.

Results are shown in FIGS. 17A-17C at 40× magnification (Olympus VS200 ASW 4.1) with a scale bar sized at 200 um shown in the lower right hand corner of each image, in which FIG. 17A shows staining from the 50 ug treated group, FIG. 17B shows staining from the 250 ug treated group, and FIG. 17C shows staining from the untreated control group. No anatomical or cellular morphological abnormalities are observed in either of the treated groups, and histopathological analysis is consistent with the untreated group. There is no sign of immune cell infiltration, necrosis, apoptosis, hemorrhage, or red blood cells extravasation within the tissues, and there is no evidence for de-nucleated cells, or loss of cellular structures. The large white sections within the tissue are normal vascular features of the liver (e.g., veins, arteries, sinusoids) and are also present in naive samples.

### Example XIII.: Sonoporation Using Ultrasound Inducing Shear Waves in the NHP Liver

In this example, application of sonoporation in a single treatment utilizing ultrasound to apply an acoustic radiation force and induce shear waves in a NHP liver to deliver green human clotting factor VIII (hFVIII) was investigated. One male cynomolgus macaque (cyno), the cyno was infused with phospholipid stabilized sonoactive microstructures (Sonazoid), and 10 milligrams of nucleic acids encoding hFVIII under the influence of an APOE-AAT promoter (APOE-AAT-hFVIII) via an intravenous infusion into the hepatic vein via a flow-directed balloon-tipped catheter inserted through the femoral vein or the jugular vein. Once advanced to the hepatic vein in the liver, the balloon-tipped catheter was inflated to occlude the flow of blood out of the liver. Approximately 1-2 mL infusions of Sonazoid sonoactive agent and nucleic acid solution were administered every 15-30 seconds. The liver of the subject was treated using ultrasound to apply an acoustic radiation force and induce shear waves in the liver tissue of the subject. The subject was administered 10 mg of the APOE-AAT-hFVIII plasmid total and applied apply an acoustic radiation force to the liver following visualization of the liver and verifying presence of the ultrasound contrast agent by attenuation of the ultrasound signal in the B-mode image. Ultrasound energy was applied using a C-16 probe on a GE LOGIQ E10 system in research mode for the acoustic radiation force ultrasound application. The ultrasound parameters are described below.

The B-mode ultrasound was applied at focal depth setting was set to 5 cm, and the zoom to 0. Ultrasound was delivered at a mechanical index (MI) value of 0.09 and at a frequency of 2.5 MHz to visual the liver tissue and confirm the presence of the sonoactive agent in the tissue and vasculature.

The acoustic radiation force ultrasound was applied to the liver region of each subject with the same GE LOGIQ e10 ultrasound system, equipped with a C1-6 probe operating using the “ELASTO” software, Shear Wave mode. The ARF was applied immediately following the visualization of the organ with the B-mode ultrasound. The acoustic radiation force was applied with ultrasound acoustic energy applied at an ultrasound intensity of at least 180 mW/cm² (I_{SPTA}) (spatial-peak temporal average intensity), a frequency of 2.5 MHz, at a mechanical index of 2.1, and tracking output reduced to about 0%, at a pulse length of about 600 us, a pulse repetition frequency of about 0.9 Hz, a pulse repetition period of about 1.1 s and a duty cycle of about 0.05%. Upon disruption of the sonoactive agent with application the ARF ultrasound, serial bolus injections of approximately 1.0-4.0 mL of sonoactive microstructure and DNA solution were re-administered to the subject about every 30 seconds, and the acoustic radiation force ultrasound was applied until the 10 mg of DNA and sonoactive agent solution was fully administered, with the total infusion and sonication treatment time occurring over about 10 minutes. A rapid loss of ultrasound contrast (due to inertial cavitation of the sonoactive agent) occurred following the application of the acoustic radiation force.

4 hours following the treatment of the study, necropsy was performed, and samples from the treated liver were collected. Liver samples were bisected and cut into three cross section to capture all layers. Samples were placed into neutral 10% formalin for ddPCR assay and transferred to 70% EtOH 20 hours post-fixation.

Samples were evaluated using digital droplet polymer chain reaction (ddPCR) to assess copy number per diploid genome (CN/DG) levels of DNA payload delivered to the cells. The procedure began by preparing the Master Mix (MM) for each reaction. First, the ddPCR Supermix for probes (no dUTP) was thawed and vortexed for at least 30 seconds. For each primer/probe set, 11 μL ddPCR Supermix, 1.1 μL primer/probe mix, 0.275 μL HindIII enzyme, and water were combined to bring the final reaction volume to 18 μL. For single-plex reactions, 5.626 μL of water was added, while for duplex reactions, 4.525 μL was added. The Master Mix was calculated with a correction factor of 1.1 to account for pipetting error. After preparation, 18 μL of the Master Mix was dispensed into individual wells of a 96-well PCR plate, which was then set aside at room temperature. DNA samples were diluted using nuclease-free water to the desired concentration, and 4 μL of the diluted genomic DNA (gDNA) or ddFHO (for negative controls) was added to each well according to the plate map. The plate was then sealed with a pierceable foil seal, ensuring the red stripe faced up at the top, and vortexed for 2 minutes at room temperature. Following this, the plate was spun down at 100-300 rcf for 1 minute to remove bubbles. The sealed plate was placed in the Automated Droplet Generator (ADG), ensuring that the destination plate on a cooling block, DG32 cartridges, ddPCR tips (with lids removed), and an empty waste container were properly positioned. The droplet generation process was initiated, and the corresponding ddPCR program was selected on the thermal cycler, which was paused while waiting for the droplet generation to finish. Once the droplet generation was completed, the 96-well plate was removed from the cooling block, resealed with a pierceable foil seal, and transferred to the thermal cycler, where the cycling process was resumed. The cooling block was returned to −20° C. in an upside-down position for future use. Thermal cycling was performed based on the ddPCR program, which typically included steps such as denaturation, annealing, and extension, although specific conditions depended on the primer/probe set used. After thermal cycling, the plate was transferred to the Droplet Reader. In the ddPCR software, the appropriate Supermix and fluorophores (e.g., FAM, VIC, HEX) were selected before initiating the droplet reading process. Once the droplet reading was completed, the results provided the quantification of plasmid DNA copy number per diploid genome, as well as its relative abundance to reference genes. The software generated data based on the fluorescence signals detected in the droplets, enabling accurate measurement of the target gene copy numbers.

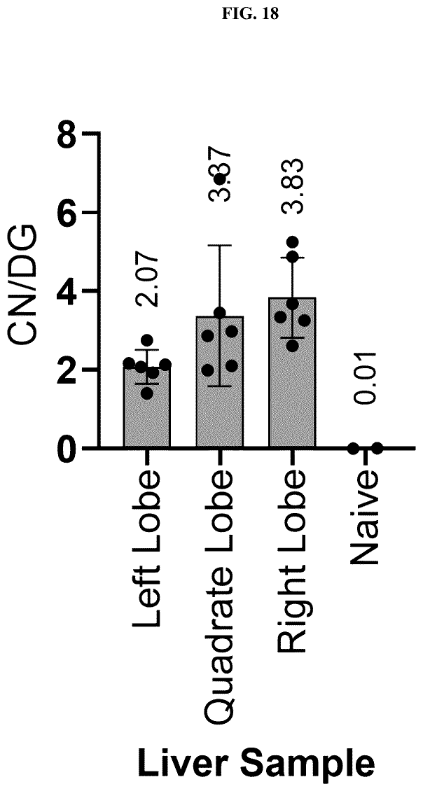

Results are shown in FIG. 18, where it is observed that the liver receiving application of the ultrasound applying the acoustic radiation force exhibited delivery of the APOE-AAT-hFVIII plasmid in each lobe of the treated liver (left lobe, quadrate lobe, right lobe) exhibiting an average copy number per diploid genome (CN/DG) ranging from 2.07 to about 3.83 CN/DG. The high copy numbers present at the 4 hour post treatment time point indicates the high level of payload delivery to the target tissue resulting from application of the acoustic radiation force inducing shear waves in the tissue by application of the ultrasound.

Results in CN/DG can be converted to copy number per nanogram of DNA (CN/ng) by use of the conversion factor of 0.006 ng of DNA per diploid genome. Applying this conversion factor, an average copy number per ng of genomic DNA (CN/ng) of about 345 CN/ng for the left lobe, 561.66 CN/ng for the quadrate lobe, and 563.33 CN/ng is obtained with respect to the treated tissue samples receiving application of the acoustic radiation force ultrasound.

### Example XIV.: Sonoporation Using B-Mode Ultrasound in the Murine Liver Evaluated for Biodistribution Using Copy Number Analysis

In this example, 3 C57BL/6 mice were studied in an experiment evaluating gene expression and biodistribution of gene expression in the mouse liver. Mice were infused with lipid stabilized sonoactive microstructures (Sonazoid) and nucleic acids encoding a luciferase reporter gene coupled to a CAG promoter (pDNA-luc) (see, e.g., SEQ ID NOS. 3-4) were administered via a surgically implanted jugular vein catheter (JVC).

In a treatment session, each mice was administered about 120 μL of sonoactive microstructures and 250 μg pDNA-luc. An acoustic contact agent (Aqua gel) was directly applied to the abdominal surface and the ultrasound acoustic energy was applied to the upper abdominal skin surface of the mice. Ultrasound was applied using a GE LOGIQ linear array probe L6-24 to generate a B-mode ultrasound image using an MI of 0.3, then alternating mechanical indexes of 0.3 and 1.5, at a frequency of 7.87 MHz was applied to the abdomen of the subjects for about 100 seconds. Low MI imaging (0.3) the liver was initiated for the initial 20 seconds following the infusion. The frequency of the low MI ultrasound was 7.87 MHz, and the pulse duration of the low MI ultrasound was 0.32 us. At 21 seconds, an ultrasound pulse at a high MI of 1.5 was applied for a pulse duration of 0.82 μs. The frequency of the high MI ultrasound was 6.2 MHz, and the pulse duration of the low MI ultrasound was 0.82 us. After the High MI mode, a Low MI imaging ultrasound was reapplied, and the High MI was implemented every 10 seconds for 9 times (total of about 90 seconds).

The treatment session was repeated twice at 48 hours between treatments, for a total of 3 treatment sessions. 72 hours following the final treatment session, necropsy was performed and treated livers were sectioned into ventral, middle, and dorsal sections for ddPCR analysis. The ventral section is the section closest to the ultrasound probe and towards to front of the abdomen of the subject, the dorsal section is the section furthest from the ultrasound probe and closest to the back of the subject, and the middle section is between the ventral and dorsal sections. Of note is that the ultrasound probe was placed over the over the ventral surface of the liver (closest physical location), whereas the middle and dorsal regions of the liver were physically removed from the transducer and the axial ultrasound beam.

Biodistribution in the treated tissue is assed using digital droplet polymer chain reaction (ddPCR) to assess copy number per diploid genome (CN/DG) levels of nucleic acid DNA payload in treated ventral, middle, and dorsal sections of the organ. The tissue was cleared of blood by sterile saline. Tissue was placed in Nase/DNase-free tubes (purchased certified or autoclaved) and snap-frozen in LN2 and transferred to −80 C storage. The procedure began by preparing the Master Mix (MM) for each reaction. First, the ddPCR Supermix for probes (no dUTP) was thawed and vortexed for at least 30 seconds. For each primer/probe set, 11 μL ddPCR Supermix, 1.1 μL primer/probe mix, 0.275 μL HindIII enzyme, and water were combined to bring the final reaction volume to 18 μL. For single-plex reactions, 5.626 μL of water was added, while for duplex reactions, 4.525 μL was added. The Master Mix was calculated with a correction factor of 1.1 to account for pipetting error. After preparation, 18 μL of the Master Mix was dispensed into individual wells of a 96-well PCR plate, which was then set aside at room temperature. DNA samples were diluted using nuclease-free water to the desired concentration, and 4 μL of the diluted genomic DNA (gDNA) or ddH₂O (for negative controls) was added to each well according to the plate map. The plate was then sealed with a pierceable foil seal, ensuring the red stripe faced up at the top, and vortexed for 2 minutes at room temperature. Following this, the plate was spun down at 100-300 rcf for 1 minute to remove bubbles. The sealed plate was placed in the Automated Droplet Generator (ADG), ensuring that the destination plate on a cooling block, DG32 cartridges, ddPCR tips (with lids removed), and an empty waste container were properly positioned. The droplet generation process was initiated, and the corresponding ddPCR program was selected on the thermal cycler, which was paused while waiting for the droplet generation to finish. Once the droplet generation was completed, the 96-well plate was removed from the cooling block, resealed with a pierceable foil seal, and transferred to the thermal cycler, where the cycling process was resumed. The cooling block was returned to −20° C. in an upside-down position for future use. Thermal cycling was performed based on the ddPCR program, which typically included steps such as denaturation, annealing, and extension, although specific conditions depended on the primer/probe set used. After thermal cycling, the plate was transferred to the Droplet Reader. In the ddPCR software, the appropriate Supermix and fluorophores (e.g., FAM, VIC, HEX) were selected before initiating the droplet reading process. Once the droplet reading was completed, the results provided the quantification of DNA payload copy number per diploid genome, as well as its relative abundance to reference genes. The software generated data based on the fluorescence signals detected in the droplets, enabling accurate measurement of the target gene copy numbers.

Results are shown in FIG. 19, in which it is shown that an average copy number of about 0.04 CN/DG was exhibited in the ventral section of the liver closest to the probe, while the middle section exhibited an average copy number of about 0.02 CN/DG, while the dorsal section exhibited an average copy number of less than 0.02 CN/DG.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCE LISTING

| NO. | Desc. | Sequence |
|---|---|---|
| 1. | Factor VIII | ATGCAGATTGAGCTGAGCACCTGCTTCTTCCTGTGCCTGCTGAGGTTCTG |
| | NT | CTTCTCTGCCACCAGGAGATACTACCTGGGGGCTGTGGAGCTGAGCTGGG |
| | | ACTACATGCAGTCTGACCTGGGGGAGCTGCCTGTGGATGCCAGGTTCCCC |
| | | CCCAGAGTGCCCAAGAGCTTCCCCTTCAACACCTCTGTGGTGTACAAGAA |
| | | GACCCTGTTTGTGGAGTTCACTGACCACCTGTTCAACATTGCCAAGCCCA |
| | | GGCCCCCCTGGATGGGCCTGCTGGGCCCCACCATCCAGGCTGAGGTGTAT |
| | | GACACTGTGGTGATCACCCTGAAGAACATGGCCAGCCACCCTGTGAGCCT |
| | | GCATGCTGTGGGGGTGAGCTACTGGAAGGCCTCTGAGGGGGCTGAGTATG |
| | | ATGACCAGACCAGCCAGAGGGAGAAGGAGGATGACAAGGTGTTCCCTGGG |
| | | GGCAGCCACACCTATGTGTGGCAGGTGCTGAAGGAGAATGGCCCCATGGC |
| | | CTCTGACCCCCTGTGCCTGACCTACAGCTACCTGAGCCATGTGGACCTGG |
| | | TGAAGGACCTGAACTCTGGCCTGATTGGGGCCCTGCTGGTGTGCAGGGAG |
| | | GGCAGCCTGGCCAAGGAGAAGACCCAGACCCTGCACAAGTTCATCCTGCT |
| | | GTTTGCTGTGTTTGATGAGGGCAAGAGCTGGCACTCTGAAACCAAGAACA |
| | | GCCTGATGCAGGACAGGGATGCTGCCTCTGCCAGGGCCTGGCCCAAGATG |
| | | CACACTGTGAATGGCTATGTGAACAGGAGCCTGCCTGGCCTGATTGGCTG |
| | | CCACAGGAAGTCTGTGTACTGGCATGTGATTGGCATGGGCACCACCCCTG |
| | | AGGTGCACAGCATCTTCCTGGAGGGCCACACCTTCCTGGTCAGGAACCAC |
| | | AGGCAGGCCAGCCTGGAGATCAGCCCCATCACCTTCCTGACTGCCCAGAC |
| | | CCTGCTGATGGACCTGGGCCAGTTCCTGCTGTTCTGCCACATCAGCAGCC |
| | | ACCAGCATGATGGCATGGAGGCCTATGTGAAGGTGGACAGCTGCCCTGAG |
| | | GAGCCCCAGCTGAGGATGAAGAACAATGAGGAGGCTGAGGACTATGATGA |
| | | TGACCTGACTGACTCTGAGATGGATGTGGTGAGGTTTGATGATGACAACA |
| | | GCCCCAGCTTCATCCAGATCAGGTCTGTGGCCAAGAAGCACCCCAAGACC |
| | | TGGGTGCACTACATTGCTGCTGAGGAGGAGGACTGGGACTATGCCCCCCT |
| | | GGTGCTGGCCCCTGATGACAGGAGCTACAAGAGCCAGTACCTGAACAATG |
| | | GCCCCCAGAGGATTGGCAGGAAGTACAAGAAGGTCAGGTTCATGGCCTAC |
| | | ACTGATGAAACCTTCAAGACCAGGGAGGCCATCCAGCATGAGTCTGGCAT |
| | | CCTGGGCCCCCTGCTGTATGGGGAGGTGGGGGACACCCTGCTGATCATCT |
| | | TCAAGAACCAGGCCAGCAGGCCCTACAACATCTACCCCCATGGCATCACT |
| | | GATGTGAGGCCCCTGTACAGCAGGAGGCTGCCCAAGGGGGTGAAGCACCT |
| | | GAAGGACTTCCCCATCCTGCCTGGGGAGATCTTCAAGTACAAGTGGACTG |
| | | TGACTGTGGAGGATGGCCCCACCAAGTCTGACCCCAGGTGCCTGACCAGA |
| | | TACTACAGCAGCTTTGTGAACATGGAGAGGGACCTGGCCTCTGGCCTGAT |
| | | TGGCCCCCTGCTGATCTGCTACAAGGAGTCTGTGGACCAGAGGGGCAACC |
| | | AGATCATGTCTGACAAGAGGAATGTGATCCTGTTCTCTGTGTTTGATGAG |
| | | AACAGGAGCTGGTACCTGACTGAGAACATCCAGAGGTTCCTGCCCAACCC |
| | | TGCTGGGGTGCAGCTGGAGGACCCTGAGTTCCAGGCCAGCAACATCATGC |
| | | ACAGCATCAATGGCTATGTGTTTGACAGCCTGCAGCTGTCTGTGTGCCTG |
| | | CATGAGGTGGCCTACTGGTACATCCTGAGCATTGGGGCCCAGACTGACTT |
| | | CCTGTCTGTGTTCTTCTCTGGCTACACCTTCAAGCACAAGATGGTGTATG |
| | | AGGACACCCTGACCCTGTTCCCCTTCTCTGGGGAGACTGTGTTCATGAGC |
| | | ATGGAGAACCCTGGCCTGTGGATTCTGGGCTGCCACAACTCTGACTTCAG |
| | | GAACAGGGGCATGACTGCCCTGCTGAAAGTCTCCAGCTGTGACAAGAACA |
| | | CTGGGGACTACTATGAGGACAGCTATGAGGACATCTCTGCCTACCTGCTG |
| | | AGCAAGAACAATGCCATTGAGCCCAGGAGCTTCAGCCAGAATGCCACTAA |
| | | TGTGTCTAACAACAGCAACACCAGCAATGACAGCAATGTGTCTCCCCCAG |
| | | TGCTGAAGAGGCACCAGAGGGAGATCACCAGGACCACCCTGCAGTCTGAC |
| | | CAGGAGGAGATTGACTATGATGACACCATCTCTGTGGAGATGAAGAAGGA |
| | | GGACTTTGACATCTACGACGAGGACGAGAACCAGAGCCCCAGGAGCTTCC |
| | | AGAAGAAGACCAGGCACTACTTCATTGCTGCTGTGGAGAGGCTGTGGGAC |
| | | TATGGCATGAGCAGCAGCCCCCATGTGCTGAGGAACAGGGCCCAGTCTGG |
| | | CTCTGTGCCCCAGTTCAAGAAGGTGGTGTTCCAGGAGTTCACTGATGGCA |
| | | GCTTCACCCAGCCCCTGTACAGAGGGGAGCTGAATGAGCACCTGGGCCTG |
| | | CTGGGCCCCTACATCAGGGCTGAGGTGGAGGACAACATCATGGTGACCTT |
| | | CAGGAACCAGGCCAGCAGGCCCTACAGCTTCTACAGCAGCCTGATCAGCT |
| | | ATGAGGAGGACCAGAGGCAGGGGGCTGAGCCCAGGAAGAACTTTGTGAAG |
| | | CCCAATGAAACCAAGACCTACTTCTGGAAGGTGCAGCACCACATGGCCCC |
| | | CACCAAGGATGAGTTTGACTGCAAGGCCTGGGCCTACTTCTCTGATGTGG |
| | | ACCTGGAGAAGGATGTGCACTCTGGCCTGATTGGCCCCCTGCTGGTGTGC |
| | | CACACCAACACCCTGAACCCTGCCCATGGCAGGCAGGTGACTGTGCAGGA |
| | | GTTTGCCCTGTTCTTCACCATCTTTGATGAAACCAAGAGCTGGTACTTCA |
| | | CTGAGAACATGGAGAGGAACTGCAGGGCCCCCTGCAACATCCAGATGGAG |
| | | GACCCCACCTTCAAGGAGAACTACAGGTTCCATGCCATCAATGGCTACAT |
| | | CATGGACACCCTGCCTGGCCTGGTGATGGCCCAGGACCAGAGGATCAGGT |
| | | GGTACCTGCTGAGCATGGGCAGCAATGAGAACATCCACAGCATCCACTTC |
| | | TCTGGCCATGTGTTCACTGTGAGGAAGAAGGAGGAGTACAAGATGGCCCT |
| | | GTACAACCTGTACCCTGGGGTGTTTGAGACTGTGGAGATGCTGCCCAGCA |
| | | AGGCTGGCATCTGGAGGGTGGAGTGCCTGATTGGGGAGCACCTGCATGCT |
| | | GGCATGAGCACCCTGTTCCTGGTGTACAGCAACAAGTGCCAGACCCCCCT |
| | | GGGCATGGCCTCTGGCCACATCAGGGACTTCCAGATCACTGCCTCTGGCC |
| | | AGTATGGCCAGTGGGCCCCCAAGCTGGCCAGGCTGCACTACTCTGGCAGC |
| | | ATCAATGCCTGGAGCACCAAGGAGCCCTTCAGCTGGATCAAGGTGGACCT |
| | | GCTGGCCCCCATGATCATCCATGGCATCAAGACCCAGGGGGCCAGGCAGA |
| | | AGTTCAGCAGCCTGTACATCAGCCAGTTCATCATCATGTACAGCCTGGAT |
| | | GGCAAGAAGTGGCAGACCTACAGGGGCAACAGCACTGGCACCCTGATGGT |
| | | GTTCTTTGGCAATGTGGACAGCTCTGGCATCAAGCACAACATCTTCAACC |
| | | CCCCCATCATTGCCAGATACATCAGGCTGCACCCCACCCACTACAGCATC |
| | | AGGAGCACCCTGAGGATGGAGCTGATGGGCTGTGACCTGAACAGCTGCAG |
| | | CATGCCCCTGGGCATGGAGAGCAAGGCCATCTCTGATGCCCAGATCACTG |
| | | CCAGCAGCTACTTCACCAACATGTTTGCCACCTGGAGCCCCAGCAAGGCC |
| | | AGGCTGCACCTGCAGGGCAGGAGCAATGCCTGGAGGCCCCAGGTCAACAA |
| | | CCCCAAGGAGTGGCTGCAGGTGGACTTCCAGAAGACCATGAAGGTGACTG |
| | | GGGTGACCACCCAGGGGGTGAAGAGCCTGCTGACCAGCATGTATGTGAAG |
| | | GAGTTCCTGATCAGCAGCAGCCAGGATGGCCACCAGTGGACCCTGTTCTT |
| | | CCAGAATGGCAAGGTGAAGGTGTTCCAGGGCAACCAGGACAGCTTCACCC |
| | | CTGTGGTGAACAGCCTGGACCCCCCCCTGCTGACCAGATACCTGAGGATT |
| | | CACCCCCAGAGCTGGGTGCACCAGATTGCCCTGAGGATGGAGGTGCTGGG |
| | | CTGTGAGGCCCAGGACCTGTA |
| 2. | FVIII AA | MQIELSTCFFLCLLRFCFSATRRYYLGAVELSWDYMQSDLGELPVDARFP |
| | | PRVPKSFPFNTSVVYKKTLFVEFTDHLFNIAKPRPPWMGLLGPTIQAEVY |
| | | DTVVITLKNMASHPVSLHAVGVSYWKASEGAEYDDQTSQREKEDDKVFPG |
| | | GSHTYVWQVLKENGPMASDPLCLTYSYLSHVDLVKDLNSGLIGALLVCRE |
| | | GSLAKEKTQTLHKFILLFAVFDEGKSWHSETKNSLMQDRDAASARAWPKM |
| | | HTVNGYVNRSLPGLIGCHRKSVYWHVIGMGTTPEVHSIFLEGHTFLVRNH |
| | | RQASLEISPITFLTAQTLLMDLGQFLLFCHISSHQHDGMEAYVKVDSCPE |
| | | EPQLRMKNNEEAEDYDDDLTDSEMDVVRFDDDNSPSFIQIRSVAKKHPKT |
| | | WVHYIAAEEEDWDYAPLVLAPDDRSYKSQYLNNGPQRIGRKYKKVRFMAY |
| | | TDETFKTREAIQHESGILGPLLYGEVGDTLLIIFKNQASRPYNIYPHGIT |
| | | DVRPLYSRRLPKGVKHLKDFPILPGEIFKYKWTVTVEDGPTKSDPRCLTR |
| | | YYSSFVNMERDLASGLIGPLLICYKESVDQRGNQIMSDKRNVILFSVFDE |
| | | NRSWYLTENIQRFLPNPAGVQLEDPEFQASNIMHSINGYVFDSLQLSVCL |
| | | HEVAYWYILSIGAQTDFLSVFFSGYTFKHKMVYEDTLTLFPFSGETVFMS |
| | | MENPGLWILGCHNSDFRNRGMTALLKVSSCDKNTGDYYEDSYEDISAYLL |
| | | SKNNAIEPRSFSQNATNVSNNSNTSNDSNVSPPVLKRHQREITRTTLQSD |
| | | QEEIDYDDTISVEMKKEDFDIYDEDENQSPRSFQKKTRHYFIAAVERLWD |
| | | YGMSSSPHVLRNRAQSGSVPQFKKVVFQEFTDGSFTQPLYRGELNEHLGL |
| | | LGPYIRAEVEDNIMVTFRNQASRPYSFYSSLISYEEDQRQGAEPRKNFVK |
| | | PNETKTYFWKVQHHMAPTKDEFDCKAWAYFSDVDLEKDVHSGLIGPLLVC |
| | | HTNTLNPAHGRQVTVQEFALFFTIFDETKSWYFTENMERNCRAPCNIQME |
| | | DPTFKENYRFHAINGYIMDTLPGLVMAQDQRIRWYLLSMGSNENIHSIHF |
| | | SGHVFTVRKKEEYKMALYNLYPGVFETVEMLPSKAGIWRVECLIGEHLHA |
| | | GMSTLFLVYSNKCQTPLGMASGHIRDFQITASGQYGQWAPKLARLHYSGS |
| | | INAWSTKEPFSWIKVDLLAPMIIHGIKTQGARQKFSSLYISQFIIMYSLD |
| | | GKKWQTYRGNSTGTLMVFFGNVDSSGIKHNIFNPPIIARYIRLHPTHYSI |
| | | RSTLRMELMGCDLNSCSMPLGMESKAISDAQITASSYFTNMFATWSPSKA |
| | | RLHLQGRSNAWRPQVNNPKEWLQVDFQKTMKVTGVTTQGVKSLLTSMYVK |
| | | EFLISSSQDGHQWTLFFQNGKVKVFQGNQDSFTPVVNSLDPPLLTRYLRI |
| | | HPQSWVHQIALRMEVLGCEAQDL |
| 3. | CAG | GCCGCCTCGCGCCGCCCGCCCCGGCTCTGACTGACCGCGTTACTCCCACA |
| | | GGTGAGCGGGCGGGACGGCCCTTCTCCTCCGGGCTGTAATTAGCGCTTGG |
| | | TTTAATGACGGCTTGTTTCTTTTCTGTGGCTGCGTGAAAGCCTTGAGGGG |
| | | CTCCGGGAGGGCCCTTTGTGCGGGGGGAGCGGCTCGGGGGGTGCGTGCGT |
| | | GTGTGTGTGCGTGGGGAGCGCCGCGTGCGGCTCCGCGCTGCCCGGCGGCT |
| | | GTGAGCGCTGCGGGCGCGGCGCGGGGCTTTGTGCGCTCCGCAGTGTGCGC |
| | | GAGGGGAGCGCGGCCGGGGGCGGTGCCCCGCGGTGCGGGGGGGGCTGCGA |
| | | GGGGAACAAAGGCTGCGTGCGGGGTGTGTGCGTGGGGGGGTGAGCAGGGG |
| | | GTGTGGGCGCGTCGGTCGGGCTGCAACCCCCCCTGCACCCCCCTCCCCGA |
| | | GTTGCTGAGCACGGCCCGGCTTCGGGTGCGGGGCTCCGTACGGGGCGTGG |
| | | CGCGGGGCTCGCCGTGCCGGGCGGGGGGTGGCGGCAGGTGGGGGTGCCGG |
| | | GCGGGGCGGGGCCGCCTCGGGCCGGGGAGGGCTCGGGGGAGGGGCGCGGC |
| | | GGCCCCCGGAGCGCCGGCGGCTGTCGAGGCGCGGCGAGCCGCAGCCATTG |
| | | CCTTTTATGGTAATCGTGCGAGAGGGCGCAGGGACTTCCTTTGTCCCAAA |
| | | TCTGTGCGGAGCCGAAATCTGGGAGGCGCCGCCGCACCCCCTCTAGCGGG |
| | | CGCGGGGCGAAGCGGTGCGGCGCCGGCAGGAAGGAAATGGGCGGGGAGGG |
| | | CCTTCGTGCGTCGCCGCGCCGCCGTCCCCTTCTCCCTCTCCAGCCTCGGG |
| | | GCTGTCCGCGGGGGGACGGCTGCCTTCGGGGGGGACGGGGCAGGGCGGGG |
| | | TTCGGCTTCTGGCGTGTGACCGGCGGCTCTAGAGCCTCTGCTAACCATGT |
| | | TCATGCCTTCTTCTTTTTCCTACAGCTCCTGGGCAACGTGCTGGTTATTG |
| | | TGCTGTCTCATCATTTTGGCAAAGAACGCGCG |
| 4. | Fluc | ATGGAAGACGCCAAAAACATAAAGAAAGGCCCGGCGCCATTCTATCCGCT |
| | | GGAAGATGGAACCGCTGGAGAGCAACTGCATAAGGCTATGAAGAGATACG |
| | | CCCTGGTTCCTGGAACAATTGCTTTTACAGATGCACATATCGAGGTGGAC |
| | | ATCACTTACGCTGAGTACTTCGAAATGTCCGTTCGGTTGGCAGAAGCTAT |
| | | GAAACGATATGGGCTGAATACAAATCACAGAATCGTCGTATGCAGTGAAA |
| | | ACTCTCTTCAATTCTTTATGCCGGTGTTGGGCGCGTTATTTATCGGAGTT |
| | | GCAGTTGCGCCCGCGAACGACATTTATAATGAACGTGAATTGCTCAACAG |
| | | TATGGGCATTTCGCAGCCTACCGTGGTGTTCGTTTCCAAAAAGGGGTTGC |
| | | AAAAAATTTTGAACGTGCAAAAAAAGCTCCCAATCATCCAAAAAATTATT |
| | | ATCATGGATTCTAAAACGGATTACCAGGGATTTCAGTCGATGTACACGTT |
| | | CGTCACATCTCATCTACCTCCCGGTTTTAATGAATACGATTTTGTGCCAG |
| | | AGTCCTTCGATAGGGACAAGACAATTGCACTGATCATGAACTCCTCTGGA |
| | | TCTACTGGTCTGCCTAAAGGTGTCGCTCTGCCTCATAGAACTGCCTGCGT |
| | | GAGATTCTCGCATGCCAGAGATCCTATTTTTGGCAATCAAATCATTCCGG |
| | | ATACTGCGATTTTAAGTGTTGTTCCATTCCATCACGGTTTTGGAATGTTT |
| | | ACTACACTCGGATATTTGATATGTGGATTTCGAGTCGTCTTAATGTATAG |
| | | ATTTGAAGAAGAGCTGTTTCTGAGGAGCCTTCAGGATTACAAGATTCAAA |
| | | GTGCGCTGCTGGTGCCAACCCTATTCTCCTTCTTCGCCAAAAGCACTCTG |
| | | ATTGACAAATACGATTTATCTAATTTACACGAAATTGCTTCTGGTGGCGC |
| | | TCCCCTCTCTAAGGAAGTCGGGGAAGCGGTTGCCAAGAGGTTCCATCTGC |
| | | CAGGTATCAGGCAAGGATATGGGCTCACTGAGACTACATCAGCTATTCTG |
| | | ATTACACCCGAGGGGGATGATAAACCGGGCGCGGTCGGTAAAGTTGTTCC |
| | | ATTTTTTGAAGCGAAGGTTGTGGATCTGGATACCGGGAAAACGCTGGGCG |
| | | TTAATCAAAGAGGCGAACTGTGTGTGAGAGGTCCTATGATTATGTCCGGT |
| | | TATGTAAACAATCCGGAAGCGACCAACGCCTTGATTGACAAGGATGGATG |
| | | GCTACATTCTGGAGACATAGCTTACTGGGACGAAGACGAACACTTCTTCA |
| | | TCGTTGACCGCCTGAAGTCTCTGATTAAGTACAAAGGCTATCAGGTGGCT |
| | | CCCGCTGAATTGGAATCCATCTTGCTCCAACACCCCAACATCTTCGACGC |
| | | AGGTGTCGCAGGTCTTCCCGACGATGACGCCGGTGAACTTCCCGCCGCCG |
| | | TTGTTGTTTTGGAGCACGGAAAGACGATGACGGAAAAAGAGATCGTGGAT |
| | | TACGTCGCCAGTCAAGTAACAACCGCGAAAAAGTTGCGCGGAGGAGTTGT |
| | | GTTTGTGGACGAAGTACCGAAAGGTCTTACCGGAAAACTCGACGCAAGAA |
| | | AAATCAGAGAGATCCTCATAAAGGCCAAGAAGGGCGGAAAGATCGCCGTG |
| | | TAA |
| 5. | APOE- | CGCGTCTGCAGGCTCAGAGGCACACAGGAGTTTCTGGGCTCACCCTGCCC |
| | AAT-FVIII | CCTTCCAACCCCTCAGTTCCCATCCTCCAGCAGCTGTTTGTGTGCTGCCT |
| | V3 | CTGAAGTCCACACTGAACAAACTTCAGCCTACTCATGTCCCTAAAATGGG |
| | | CAAACATTGCAAGCAGCAAACAGCAAACACACAGCCCTCCCTGCCTGCTG |
| | | ACCTTGGAGCTGGGGCAGAGGTCAGAGACCTCTCTGGGCCCATGCCACCT |
| | | CCAACATCCACTCGACCCCTTGGAATTTCGGTGGAGAGGAGCAGAGGTTG |
| | | TCCTGGCGTGGTTTAGGTAGTGTGAGAGGGGTCGACTGGACACAGGACGC |
| | | TGTGGTTTCTGAGCCAGGGGGCGACTCAGATCCCAGCCAGTGGACTTAGC |
| | | CCCTGTTTGCTCCTCCGATAACTGGGGTGACCTTGGTTAATATTCACCAG |
| | | CAGCCTCCCCCGTTGCCCCTCTGGATCCACTGCTTAAATACGGACGAGGA |
| | | CAGGGCCCTGTCTCCTCAGCTTCAGGCACCACCACTGACCTGGGACAGTG |
| | | AATCGTAAGTACTAGCAGCTACAATCCAGCTACCATTCTGCTTTTATTTT |
| | | ATGGTTGGGATAAGGCTGGATTATTCTGAGTCCAAGCTAGGCCCTTTTGC |
| | | TAATCATGTTCATACCTCTTATCTTCCTCCCACAGCTCCTGGGCAACGTG |
| | | CTGGTCTGTGTGCTGGCCCATCACTTTGGCAAAGAATTGCGATCGCCACC |
| | | ATGCAGATTGAGCTGAGCACCTGCTTCTTCCTGTGCCTGCTGAGGTTCTG |
| | | CTTCTCTGCCACCAGGAGATACTACCTGGGGGCTGTGGAGCTGAGCTGGG |
| | | ACTACATGCAGTCTGACCTGGGGGAGCTGCCTGTGGATGCCAGGTTCCCC |
| | | CCCAGAGTGCCCAAGAGCTTCCCCTTCAACACCTCTGTGGTGTACAAGAA |
| | | GACCCTGTTTGTGGAGTTCACTGACCACCTGTTCAACATTGCCAAGCCCA |
| | | GGCCCCCCTGGATGGGCCTGCTGGGCCCCACCATCCAGGCTGAGGTGTAT |
| | | GACACTGTGGTGATCACCCTGAAGAACATGGCCAGCCACCCTGTGAGCCT |
| | | GCATGCTGTGGGGGTGAGCTACTGGAAGGCCTCTGAGGGGGCTGAGTATG |
| | | ATGACCAGACCAGCCAGAGGGAGAAGGAGGATGACAAGGTGTTCCCTGGG |
| | | GGCAGCCACACCTATGTGTGGCAGGTGCTGAAGGAGAATGGCCCCATGGC |
| | | CTCTGACCCCCTGTGCCTGACCTACAGCTACCTGAGCCATGTGGACCTGG |
| | | TGAAGGACCTGAACTCTGGCCTGATTGGGGCCCTGCTGGTGTGCAGGGAG |
| | | GGCAGCCTGGCCAAGGAGAAGACCCAGACCCTGCACAAGTTCATCCTGCT |
| | | GTTTGCTGTGTTTGATGAGGGCAAGAGCTGGCACTCTGAAACCAAGAACA |
| | | GCCTGATGCAGGACAGGGATGCTGCCTCTGCCAGGGCCTGGCCCAAGATG |
| | | CACACTGTGAATGGCTATGTGAACAGGAGCCTGCCTGGCCTGATTGGCTG |
| | | CCACAGGAAGTCTGTGTACTGGCATGTGATTGGCATGGGCACCACCCCTG |
| | | AGGTGCACAGCATCTTCCTGGAGGGCCACACCTTCCTGGTCAGGAACCAC |
| | | AGGCAGGCCAGCCTGGAGATCAGCCCCATCACCTTCCTGACTGCCCAGAC |
| | | CCTGCTGATGGACCTGGGCCAGTTCCTGCTGTTCTGCCACATCAGCAGCC |
| | | ACCAGCATGATGGCATGGAGGCCTATGTGAAGGTGGACAGCTGCCCTGAG |
| | | GAGCCCCAGCTGAGGATGAAGAACAATGAGGAGGCTGAGGACTATGATGA |
| | | TGACCTGACTGACTCTGAGATGGATGTGGTGAGGTTTGATGATGACAACA |
| | | GCCCCAGCTTCATCCAGATCAGGTCTGTGGCCAAGAAGCACCCCAAGACC |
| | | TGGGTGCACTACATTGCTGCTGAGGAGGAGGACTGGGACTATGCCCCCCT |
| | | GGTGCTGGCCCCTGATGACAGGAGCTACAAGAGCCAGTACCTGAACAATG |
| | | GCCCCCAGAGGATTGGCAGGAAGTACAAGAAGGTCAGGTTCATGGCCTAC |
| | | ACTGATGAAACCTTCAAGACCAGGGAGGCCATCCAGCATGAGTCTGGCAT |
| | | CCTGGGCCCCCTGCTGTATGGGGAGGTGGGGGACACCCTGCTGATCATCT |
| | | TCAAGAACCAGGCCAGCAGGCCCTACAACATCTACCCCCATGGCATCACT |
| | | GATGTGAGGCCCCTGTACAGCAGGAGGCTGCCCAAGGGGGTGAAGCACCT |
| | | GAAGGACTTCCCCATCCTGCCTGGGGAGATCTTCAAGTACAAGTGGACTG |
| | | TGACTGTGGAGGATGGCCCCACCAAGTCTGACCCCAGGTGCCTGACCAGA |
| | | TACTACAGCAGCTTTGTGAACATGGAGAGGGACCTGGCCTCTGGCCTGAT |
| | | TGGCCCCCTGCTGATCTGCTACAAGGAGTCTGTGGACCAGAGGGGCAACC |
| | | AGATCATGTCTGACAAGAGGAATGTGATCCTGTTCTCTGTGTTTGATGAG |
| | | AACAGGAGCTGGTACCTGACTGAGAACATCCAGAGGTTCCTGCCCAACCC |
| | | TGCTGGGGTGCAGCTGGAGGACCCTGAGTTCCAGGCCAGCAACATCATGC |
| | | ACAGCATCAATGGCTATGTGTTTGACAGCCTGCAGCTGTCTGTGTGCCTG |
| | | CATGAGGTGGCCTACTGGTACATCCTGAGCATTGGGGCCCAGACTGACTT |
| | | CCTGTCTGTGTTCTTCTCTGGCTACACCTTCAAGCACAAGATGGTGTATG |
| | | AGGACACCCTGACCCTGTTCCCCTTCTCTGGGGAGACTGTGTTCATGAGC |
| | | ATGGAGAACCCTGGCCTGTGGATTCTGGGCTGCCACAACTCTGACTTCAG |
| | | GAACAGGGGCATGACTGCCCTGCTGAAAGTCTCCAGCTGTGACAAGAACA |
| | | CTGGGGACTACTATGAGGACAGCTATGAGGACATCTCTGCCTACCTGCTG |
| | | AGCAAGAACAATGCCATTGAGCCCAGGAGCTTCAGCCAGAATGCCACTAA |
| | | TGTGTCTAACAACAGCAACACCAGCAATGACAGCAATGTGTCTCCCCCAG |
| | | TGCTGAAGAGGCACCAGAGGGAGATCACCAGGACCACCCTGCAGTCTGAC |
| | | CAGGAGGAGATTGACTATGATGACACCATCTCTGTGGAGATGAAGAAGGA |
| | | GGACTTTGACATCTACGACGAGGACGAGAACCAGAGCCCCAGGAGCTTCC |
| | | AGAAGAAGACCAGGCACTACTTCATTGCTGCTGTGGAGAGGCTGTGGGAC |
| | | TATGGCATGAGCAGCAGCCCCCATGTGCTGAGGAACAGGGCCCAGTCTGG |
| | | CTCTGTGCCCCAGTTCAAGAAGGTGGTGTTCCAGGAGTTCACTGATGGCA |
| | | GCTTCACCCAGCCCCTGTACAGAGGGGAGCTGAATGAGCACCTGGGCCTG |
| | | CTGGGCCCCTACATCAGGGCTGAGGTGGAGGACAACATCATGGTGACCTT |
| | | CAGGAACCAGGCCAGCAGGCCCTACAGCTTCTACAGCAGCCTGATCAGCT |
| | | ATGAGGAGGACCAGAGGCAGGGGGCTGAGCCCAGGAAGAACTTTGTGAAG |
| | | CCCAATGAAACCAAGACCTACTTCTGGAAGGTGCAGCACCACATGGCCCC |
| | | CACCAAGGATGAGTTTGACTGCAAGGCCTGGGCCTACTTCTCTGATGTGG |
| | | ACCTGGAGAAGGATGTGCACTCTGGCCTGATTGGCCCCCTGCTGGTGTGC |
| | | CACACCAACACCCTGAACCCTGCCCATGGCAGGCAGGTGACTGTGCAGGA |
| | | GTTTGCCCTGTTCTTCACCATCTTTGATGAAACCAAGAGCTGGTACTTCA |
| | | CTGAGAACATGGAGAGGAACTGCAGGGCCCCCTGCAACATCCAGATGGAG |
| | | GACCCCACCTTCAAGGAGAACTACAGGTTCCATGCCATCAATGGCTACAT |
| | | CATGGACACCCTGCCTGGCCTGGTGATGGCCCAGGACCAGAGGATCAGGT |
| | | GGTACCTGCTGAGCATGGGCAGCAATGAGAACATCCACAGCATCCACTTC |
| | | TCTGGCCATGTGTTCACTGTGAGGAAGAAGGAGGAGTACAAGATGGCCCT |
| | | GTACAACCTGTACCCTGGGGTGTTTGAGACTGTGGAGATGCTGCCCAGCA |
| | | AGGCTGGCATCTGGAGGGTGGAGTGCCTGATTGGGGAGCACCTGCATGCT |
| | | GGCATGAGCACCCTGTTCCTGGTGTACAGCAACAAGTGCCAGACCCCCCT |
| | | GGGCATGGCCTCTGGCCACATCAGGGACTTCCAGATCACTGCCTCTGGCC |
| | | AGTATGGCCAGTGGGCCCCCAAGCTGGCCAGGCTGCACTACTCTGGCAGC |
| | | ATCAATGCCTGGAGCACCAAGGAGCCCTTCAGCTGGATCAAGGTGGACCT |
| | | GCTGGCCCCCATGATCATCCATGGCATCAAGACCCAGGGGGCCAGGCAGA |
| | | AGTTCAGCAGCCTGTACATCAGCCAGTTCATCATCATGTACAGCCTGGAT |
| | | GGCAAGAAGTGGCAGACCTACAGGGGCAACAGCACTGGCACCCTGATGGT |
| | | GTTCTTTGGCAATGTGGACAGCTCTGGCATCAAGCACAACATCTTCAACC |
| | | CCCCCATCATTGCCAGATACATCAGGCTGCACCCCACCCACTACAGCATC |
| | | AGGAGCACCCTGAGGATGGAGCTGATGGGCTGTGACCTGAACAGCTGCAG |
| | | CATGCCCCTGGGCATGGAGAGCAAGGCCATCTCTGATGCCCAGATCACTG |
| | | CCAGCAGCTACTTCACCAACATGTTTGCCACCTGGAGCCCCAGCAAGGCC |
| | | AGGCTGCACCTGCAGGGCAGGAGCAATGCCTGGAGGCCCCAGGTCAACAA |
| | | CCCCAAGGAGTGGCTGCAGGTGGACTTCCAGAAGACCATGAAGGTGACTG |
| | | GGGTGACCACCCAGGGGGTGAAGAGCCTGCTGACCAGCATGTATGTGAAG |
| | | GAGTTCCTGATCAGCAGCAGCCAGGATGGCCACCAGTGGACCCTGTTCTT |
| | | CCAGAATGGCAAGGTGAAGGTGTTCCAGGGCAACCAGGACAGCTTCACCC |
| | | CTGTGGTGAACAGCCTGGACCCCCCCCTGCTGACCAGATACCTGAGGATT |
| | | CACCCCCAGAGCTGGGTGCACCAGATTGCCCTGAGGATGGAGGTGCTGGG |
| | | CTGTGAGGCCCAGGACCTGTACTGA |
| 6. | GFP | ATGGTGAGCAAGGGCGAGGAGCTGTTCACCGGGGTGGTGCCCATCCTGGT |
| | | CGAGCTGGACGGCGACGTAAACGGCCACAAGTTCAGCGTGTCCGGCGAGG |
| | | GCGAGGGCGATGCCACCTACGGCAAGCTGACCCTGAAGTTCATCTGCACC |
| | | ACCGGCAAGCTGCCCGTGCCCTGGCCCACCCTCGTGACCACCCTGACCTA |
| | | CGGCGTGCAGTGCTTCAGCCGCTACCCCGACCACATGAAGCAGCACGACT |
| | | TCTTCAAGTCCGCCATGCCCGAAGGCTACGTCCAGGAGCGCACCATCTTC |
| | | TTCAAGGACGACGGCAACTACAAGACCCGCGCCGAGGTGAAGTTCGAGGG |
| | | CGACACCCTGGTGAACCGCATCGAGCTGAAGGGCATCGACTTCAAGGAGG |
| | | ACGGCAACATCCTGGGGCACAAGCTGGAGTACAACTACAACAGCCACAAC |
| | | GTCTATATCATGGCCGACAAGCAGAAGAACGGCATCAAGGTGAACTTCAA |
| | | GATCCGCCACAACATCGAGGACGGCAGCGTGCAGCTCGCCGACCACTACC |
| | | AGCAGAACACCCCCATCGGCGACGGCCCCGTGCTGCTGCCCGACAACCAC |
| | | TACCTGAGCACCCAGTCCGCCCTGAGCAAAGACCCCAACGAGAAGCGCGA |
| | | TCACATGGTCCTGCTGGAGTTCGTGACCGCCGCCGGGATCACTCTCGGCA |
| | | TGGACGAGCTGTACAAG |
| 7. | tdTomato | ATGGTGAGCAAGGGCGAGGAGGTCATCAAAGAGTTCATGCGCTTCAAGGT |
| | | GCGCATGGAGGGCTCCATGAACGGCCACGAGTTCGAGATCGAGGGCGAGG |
| | | GCGAGGGCCGCCCCTACGAGGGCACCCAGACCGCCAAGCTGAAGGTGACC |
| | | AAGGGCGGCCCCCTGCCCTTCGCCTGGGACATCCTGTCCCCCCAGTTCAT |
| | | GTACGGCTCCAAGGCGTACGTGAAGCACCCCGCCGACATCCCCGATTACA |
| | | AGAAGCTGTCCTTCCCCGAGGGCTTCAAGTGGGAGCGCGTGATGAACTTC |
| | | GAGGACGGCGGTCTGGTGACCGTGACCCAGGACTCCTCCCTGCAGGACGG |
| | | CACGCTGATCTACAAGGTGAAGATGCGCGGCACCAACTTCCCCCCCGACG |
| | | GCCCCGTAATGCAGAAGAAGACCATGGGCTGGGAGGCCTCCACCGAGCGC |
| | | CTGTACCCCCGCGACGGCGTGCTGAAGGGCGAGATCCACCAGGCCCTGAA |
| | | GCTGAAGGACGGCGGCCACTACCTGGTGGAGTTCAAGACCATCTACATGG |
| | | CCAAGAAGCCCGTGCAACTGCCCGGCTACTACTACGTGGACACCAAGCTG |
| | | GACATCACCTCCCACAACGAGGACTACACCATCGTGGAACAGTACGAGCG |
| | | CTCCGAGGGCCGCCACCACCTGTTCCTGGGGCATGGCACCGGCAGCACCG |
| | | GCAGCGGCAGCTCCGGCACCGCCTCCTCCGAGGACAACAACATGGCCGTC |
| | | ATCAAAGAGTTCATGCGCTTCAAGGTGCGCATGGAGGGCTCCATGAACGG |
| | | CCACGAGTTCGAGATCGAGGGCGAGGGCGAGGGCCGCCCCTACGAGGGCA |
| | | CCCAGACCGCCAAGCTGAAGGTGACCAAGGGCGGCCCCCTGCCCTTCGCC |
| | | TGGGACATCCTGTCCCCCCAGTTCATGTACGGCTCCAAGGCGTACGTGAA |
| | | GCACCCCGCCGACATCCCCGATTACAAGAAGCTGTCCTTCCCCGAGGGCT |
| | | TCAAGTGGGAGCGCGTGATGAACTTCGAGGACGGCGGTCTGGTGACCGTG |
| | | ACCCAGGACTCCTCCCTGCAGGACGGCACGCTGATCTACAAGGTGAAGAT |
| | | GCGCGGCACCAACTTCCCCCCCGACGGCCCCGTAATGCAGAAGAAGACCA |
| | | TGGGCTGGGAGGCCTCCACCGAGCGCCTGTACCCCCGCGACGGCGTGCTG |
| | | AAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCG |
| | | GCTACTACTACGTGGACACCAAGCTGGACATCACCTCCCACAACGAGGAC |
| | | TACACCATCGTGGAACAGTACGAGCGCTCCGAGGGCCGCCACCACCTGTT |
| | | CCTGTACGGCATGGACGAGCTGTACAAG |

1. A method of delivering an exogenous payload to a cell in a tissue of a subject, the method comprising:
a. delivering an exogenous payload to the subject;
b. administering a sonoactive agent to the subject; and
c. applying an acoustic radiation force (ARF) to the tissue of the subject, thereby generating shear waves in the tissue of the subject, wherein the acoustic radiation force enhances delivery of the exogenous payload to the cell in the tissue of an organ of the subject, wherein the ARF is applied using ultrasound acoustic energy at an ultrasound spatial peak temporal average intensity of 100-5000 mW/cm2.

2-4. (canceled)

5. A method of distributing a payload across an organ, comprising: administering the payload and a sonoactive agent to a subject, and applying an acoustic radiation force (ARF) sufficient to deliver at least 1 copy per nanogram of subject DNA throughout the organ.

6-7. (canceled)

8. The method of claim 1, wherein the ARF is applied at duty cycle of less than 5%.

9. (canceled)

10. The method of claim 1, wherein the ARF is applied at an ultrasound pulse length of at least 20 us.

11. The method of claim 5, wherein the payload is an exogenous payload, and wherein the ARF generates shear waves in a tissue of the organ of the subject.

12-13. (canceled)

14. The method of claim 5, wherein the payload is expressed throughout the organ in two samples of the organ taken from opposite ends of the organ.

15. The method of claim 14, wherein the samples are samples sized 1 cm³ or 1 g.

16. The method of claim 14, wherein the payload is expressed in cells in the two samples at an expression level of at least 0.005 copies per diploid genome.

17-29. (canceled)

30. The method of claim 1, wherein the ARF is applied at an ultrasound pulse length 200-5000 microseconds (us).

31-51. (canceled)

52. The method of claim 1, wherein the ARF is applied at a pulse repetition frequency of 0.5 to 5 Hz.

53-56. (canceled)

57. The method of claim 1, wherein the ARF is applied at a mechanical index of greater than 0.4 up to about 3.0.

58-63. (canceled)

64. The method claim 1, wherein the ARF is applied to the tissue at a duty cycle of 0.01%-1.0%.

65-67. (canceled)

68. The method of claim 1, wherein the ARF is applied at a mechanical index of at least 2.1.

69. The method of claim 1, wherein the ARF is applied at a thermal index of less than 1.0.

70-73. (canceled)

74. The method of claim 1, wherein the applying the ARF does not increase a temperature of the tissue by more than 0.5 C.

75. (canceled)

76. The method of claim 1, wherein the ARF displaces the tissue of the subject by at least 0.1 mm.

77-90. (canceled)

91. The method of claim 1, wherein the exogenous payload comprises a nucleic acid construct.

92-109. (canceled)

110. The method of claim 1, wherein the acoustic radiation force is applied for at least 10, 20, 30, 60, 120, 180, 240, 300, 360, 420, 480, 540, or 600 seconds.

111-147. (canceled)

148. The method of claim 1, further comprising: delivering a second or subsequent dose of the exogenous payload to the subject; and applying a second or subsequent acoustic radiation force (ARF) to the subject at least 4 hours after ARF.

149-298. (canceled)

299. A system comprising:
a. an ultrasound transducer configured to apply a focused acoustic radiation force (ARF) to a tissue of a subject and generate shear waves in the tissue of the subject; and
b. a computer system comprising a computer processor and a computer-readable medium configured to implement a method of applying the focused ARF and generate the shear waves in the tissue of the subject by applying ultrasonic acoustic energy with the ultrasound transducer, the method comprising applying the ARF with ultrasound acoustic energy at one or more of:
i. an ultrasound intensity of 100-5000 mW/cm2;
ii. a pulse length of greater than 200 μs (microseconds) and at an ultrasound intensity of up to 5000 mW/cm2;
iii. a duty cycle of less than 5% and a pulse length of greater than 200 us;
iv. an ultrasound intensity of at least 100 mW/cm2 and duty cycle of less than 1%; or
v. combinations thereof,
wherein the subject has been administered an exogenous payload and a sonoactive agent.
